SEQUENCE LISTING (119 sequences)

SEQ ID NO: 1 - DNA, 14994 bp, synthetic construct
  misc_feature 1..14994: note = NA sequence human ANGPL3 gene
  source 1..14994: mol_type = other DNA
aatgacaaactgaaaaaatctattgtttgttatatatataacaaagaattagtatccacaatatgtaaataattcctaaaattagtcagaaagagacaaacttaaaaagagggtaacaaggaggggagcaaattatgtacataaccagatgattcgcaaagacggcaacagagatggccagcaaaacaaactagatatatacttgtctattagatttatcaacattttttgcctttttcattaaaagcatttgtaaaaggatataggaaaagaggaactctcatatactcctggcagggatgtaaattggtacaacccttttgaaggacaatctgacaaaagcaatcgtaagttacaagtcaacatctatgaatgtatatgaaaatatttatatacatacatcaccaccataaaagcattttctatacatactgtttataattagaaaattggaaacaaatgattaaaagggggctgattaaattaaggttcatctatataacaggattatgcagctattaaaaaggacgtggtaactctatagacattcataggaaaataaattttaaaatactaagatcctgaatgatatatatatcatgagctattatacataacaagatcccacttgtgttataaaaaattatgtttagtcattcaaagggtctggtatgatagacccaaaatgttaatagagtcgagatttttattttttataggtttttgaaatacctgaattttcacaataagtactttgcacattaaaaatcttagctgggcatggtggctcacgcttgtaatcccagcactctgggaggccaaggtaggcagatcacctgaggtcaggagttcgagaccagtctggccaacatggtgaaaccccgtctctactaaaaatacaaaaattagccaggcttggttgggggtgcctgtaattccagatactcgggaagctgagacaggagaatcgcttgaacccaggagggggaagttgcagtgagctgagatcacgacgctgcactccagcctgggcaacaaagagcaaaactccgtctcaaaaataaataaagaaaaaatctttacatgtccaaagatacggctgttcaactaaaaaatatatatgtataaaacttaacatgttaatagtgaacacacaaaacagtaagatagataaaattattccttcaaagctcacttaacctctggatctacactgtccaaaaagacggtctaatgagacaattgagcacttgataggtgagtggttctaactgagatatgttctcagtataaaacatacaataggatcttcctatacaacattaattaaaaaacaaactattgtagttaaaaaggaaaaaattagagatactatgtaaaaaagagccaaaataccttgtattttatttgaaagacatatctccataagattacacaacctcgtgtaggataaaggactttgctttgcttggaatttaaacaatttaggctcttaaatgtcctaaaattctctgtagctaagaaatttttatattggttcctaggaactaggaatccttaaattaggccctacatttgcttacaagtttattttccttggcataaaattttttagtttttacattactggttatatttgatcagggttctatttaaataggcacaagttcaagcaaagatcagattctgcttttagcagtgtgtactcagacaggaagtattaaaaggcaggcagaaaatcctttataaaattactactttcaatgcattttcccacgttgaaatgcttctgcagtttataattaggcaaattactttaattataatcaataatgctgttcaaattactataagaattatacagatatttataccaagagacaatatactagaaaccaagactacgtgaccattacctctactctgtcagtgttatttgtgagaaattgcacaaattttgcaaaaagtgttagtatcctactacagtaggatataatatagaaggaaataatttcataaagcctgtctttggtactagtgctcagttactttcattaactaaaaaaggggctactcttcaaattcccttctctaaaaagaatgtactatatcaaaagggggtaaacactactacgtatacattctgcacttagaaatccctatatgttgattttatcattctcttattcaataaatattgtttctacaatgtgtaaggcattactgtactaaagcattataaggaatataagttaaaaacacatacaaatcttgccaatcaacagcttatagtgtaataggggagagaagctggcccatctatattctccctcaactagcaagtggatgaaatatcagggtcaatagttataagccacaaaagctgacagcttaattaagagaagttttgaaatatgtatttcatgaccaataattacaactgtaacttttctatttaaagaaggagaaaatttgaatttcttctctagctcaacatacacttctataattccattacatgaaccagagtaaagggtaagatggaaatgaagaatattttcttacccttttgtggttctatattggacacttaaaaatcatacacaacctaatcaaaagatgtaattctttaaaaaggtacgagaccaaaattcagaaaatctagactataacaaaatttctcaatttacattatcttaatatgcaattaattttcaccagtaaaatactatagtatgggtacaaatgcattgattagttctaattacaaaaatggctaatatataatactgtgtagtgtttatgatacatcagataatgttctaagtgctctgaaaatataaacttttaatcttttatacgaccctataaaataggtgttattctcactggagagatgagaaaacaggggttcagagatgtgaagtaatttgaccaaaggtcacaaagctgaagaatatgaaatccgggattctgattcaggcagtcttattccagaatcatgctcttaaccactatggaatactgcctctactgtaactattatacccaaaacccttaatcctaagtcatcaaaaggaagagcctctattttacacaatgaagaggcatttctaagaatagaaatttagggacgagcacagtggcttactcctttaatatcagcactttgagaggctgatatgggaggttcacttgaagtcaggagttcaaggtcagcttgggcaacatagtgaaacacagtctctacaaaatatttaaaaattagctgggtgtggtggcatgcatctatagtctcagctacttgggagacagagggaggaggcttgctcgagcccaggagttcgtggctatagtgagctatgatcatgccactgcactccagcctggacaacagagcaagaccctgtctctaaaaaagaaaagaaatttggaaatggtttattttgtattaacaatttataatttacactgaaatttattatgataaaacttttccctgtgttaaaaagctattaactttatgaaaaatttcttttaggtaaggttgattatatatacccacacacatacacaggttaaaagttagtttcatgtgacataataactagcattttgagcactacctgtttgcccagcactgttctaagtgctctacatgtattattgttaaattatcataacactatgaattatgtactataattaccccagctttacagatgaggagactaatccatggggaggttaagtaacttgtccaaggccagacagctagagccggcttttggacccacaccacagtctgactccagcacccatattcttaacaatttcaccatattaatatgtcaagattaagcagttttaaaggatgctattttctcacaaatttcttaatatgaacactcaataagaataatcactaatataagcatttagtatttttttaacactaagttggaagcatagtggaacatttatttttagaaatattattaattggctgggctcacgcttgtaatcggctgggctcatgcctgtaaattttgggaggccaaggtaaaagaattgcttgagcccagtatttccagaccagcatgggcaatacattaagacatcatctttaaaaaaaaaatgttattaatctcctctttttgttaaatgtatattatcaaaattgttactaagctaacaaacttcagaaaaacttatgatgggcaagctgcttgtgacattgaaggtatttaagattcaattctagtttggtcctagatgaccacatatccattgttccttcaacgagcacatggtaaagagcctagaacacagagacacagaacacagtggagaaaagggagtgaaatgtctttaatgacacttactatatatgggattttgtgacaatatacaaggatggttaagacatataaggtgatgcaaaaaaacatattaacaattatagtgacaaaaaatgaggagcatataattatacattgatttatacagagtaccagaggaacacagcattgagagccgtaacaccacctgagggagtggagaaaggcttcagagagaaagtgttttttggaatggatcactgtttccaaaagaactaaagtacagtttgagaaatgcatacttaattcattacttttttcccctcaactttaataataaatttacccaacaaaaaagtttatttttgacttgtaaatctcttaaaatcataaaaaagtaaaattagcttttaaaaacaggtagtcaccatagcattgaatgtgtagtttataatacagcaaagttaaatacaatttcaaattacctattaagttagttgctcatttctttgatttcatttagcattgatctaactcaatgtggaagaaggttacattcgtgcaagttaacacggcttaatgattaactatgttcacctaccaaccttaccttttctgggcaaatattggtatatatagagttaagaagtctaggtctgcttccagaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaagtaagtagaaaataaagagggttcatgtttatgttttcaatgtggatcttttaaaaaaaatatttctaaggcatgccatttgaaatactttgttgcattgttgaaatacttttttttccaagaaaaataatctccagaaaataaaatttcctattataatttcaagttagttttttgtttccctaatgttatatatgaaaacactgaaaatttgcattttatatgaaaattacaaatcggttaaattatacaatctagaacactatgtcattacactattgtaaattactgaaggtaagtaaaaagttaaaaaaaatttaaaactattctccagtgtttaaaacagattaaataatacagtaaatggaaaagatttattcatatgaaaatatgctgggctttttcttttaattgaagttcagaaaatcaaattttagagatagtacaatttaaataaaatgttaaggacaaaaatatgtgctatttgaaagaagcatacaaggggaaggaattgccaatattcatttttcaaatccattattagtttaaaaatttagattatgatagtgttacaggaaattaatagaaaagaaagaggaaagcaacttataaccaacctactctctatatccagacttttgtagaaaaacaagataatagcatcaaagaccttctccagaccgtggaagaccaatataaacaattaaaccaacagcatagtcaaataaaagaaatagaaaatcaggtaagtcagtattttaatggtatgtcccatctttcacacaggtctgtaaaaacactgaatcctaaaattatttacaagctttaactggatcatgagtaaaattatcacatcagcataactgttaaaattgcaggctctgaagctaataaactacctgcatttaaaccatggctctaaaactttgtgtgaccttgaataaattacttcacccctttatctctcagtttcctcacatatactacaaagataataacagaacttataggattattgtaagaaaaaaaattaattcatagcagccaatgtcatcttactaaaattcaaattagatcatgtttctctttgctcaaaaccacacaatagctttccatttcactcatattggctctttagaccaagattacccaacccttcgtcatctcactgacttcacctcctctactctagttattctgaccgctttaccagtattcaaacacatcaaacatactgccacctcaaagcctttgcccttgttgtttcctctaactggaacgctcttctgccctggtatctacgtggcccactctctgatttcccttagggtcgttatcaaacaaaaaattcccaatgaagacttacaaggtcacttaaccaaaaatcacaaccgcctggtcccatccctgaaaacttctacttccttagctacttttctcctgcacactcacctttatttaacataacataaattttagttatttatctcttctattcctgcactaaaatgtaagctctgtgaatacagggattttttccattatcttcatattttccattatttgtatatactccagaatatagaatactgtatggcacacagtaggcatttctgttgaattaataaatgtaatgtcatattcacacagaagcgtgtgctatgattattattacttggattactagaaatagtgtgcctcataattaaaggtcaacattcaacaatgtaattaatctacaatgtaaacatctggtgaagtgacagagggaagcacttgtttagaaaaaagctatgtcagaatccatgtattctaatatgcagtacaatagtttaaaaatattaataatactctcaaacagctattcaagaggattcaaaaaacataatataaactcagagaaactggtaaacaaaatcattttcaagagatataaaacaaatattattaccaatttccactaaacaaacataatgttagtagtgctgctaaaaggttttttatcaactacttttggtttccatactttccttcttatgatgttattattctaaattcttttcaattatatcttttactatgattaaatgaacctgctccccaaagcaaaatgttactatagtaatatacattgtgtctaaaaataaaaatgtgtgaagaaaccaaaacaatgaatttctgagttggaagaagagttagatcatttaactttctcatatttaaattaaaaaaacaaaactctaaaaatttaagtaactttaagatcacatagttacttagtagaaaagagtaatacccagcaagcaaactttacaatagatccttttaaataaggtcctaggaaatatcattcatgccagcatcaaaaaactaacactaataatgcaagatattatatattctgcttttcttactgtcaatgagaaaaactatcattcaataaattgcaaacccaacacacttaaataaaaataaaatgttactgctaaactaacgataaactactgaatatatagaaagtaagcaaacaaacttgccaacctgccaacatctacagatatgtttacaggtcaaaaattatcaaattatcaagaaagcctggttcaaattatgtattatgtctttatcacaggtctgaagatcagtaagacctaaaactgaaaattattaaacttaaaatctgaacagaatatcaaatatattttattcatataaataaaagaatacattacaatattctaagcaaagcagtctctacttttggccttgctctgttttccgaccaatgtctgcttttttgccttgctttatttttttatcttattaaataatgtccctgattaaatattttgagaacaggtaatctgtacaatctgaataacactgtttatctaaatatcaaacaccgttataacattatgaactgaaagacaaactgtacttctgacatccttactcagatttcccctaattgtatattcagtatcattttaaaaaacagatttatattcttttatcagctcagaaggactagtattcaagaacccacagaaatttctctatcttccaagccaagagcaccaagaactactccctttcttcagttgaatgaaataagaaatgtaaaacatgatggtaagacactttggtgggtttccttcttgaagctattattatcaaattccctattcttaggacttgttctagactaaaagatagttaagagatatccatcaaatacaatgtatcaacctaaactggatgctggggttctttttacaccctataaaagacatacctaagacaatcagagaaatacaaatatggacttgattattagataatatagaaggtttattaattttcttagatgtgatcatggtattgcagttttaaaggagaacaatctcctgtttaagagatacatgctgaaatatttacggagttaaaggtcactggactccagactggtgatagaacaagactctgtctctaaaaaataattaattttttaaaagaaaatagtttggtaagatgattcttacattcttaaataacacgccatctaagaaaaatgctttaacataaacattactgaaaaaatgctacatttgccacaacttcataaaatgtcaagtgaaatctcaagctccaaagatattattcctattactaaatctgatgtaataacattttattgattctaggcattcctgctgaatgtaccaccatttataacagaggtgaacatacaagtggcatgtatgccatcagacccagcaactctcaagtttttcatgtctactgtgatgttatatcaggtaaaacctgtctaaggagaatagacagtagttagttcaacttactcattacgtattaggaagattaacctggttatcattgttttatacatatatatatgaaatatatatgagtattcgtataaatataatacttttaccttgtttatgtatttactcaatattctccttttcctctaaaataatctgaagtgactattatcaataagtttactatgccaaaattcattaattgcctttcacttaacttttgggaccataataaataataaaatgtattgccataacattaataaactaccttacaaaaccaccaattaaaatcaaacaaacaaaaaagtgttatttacatctgtcaacataaatctactaaaaatacatgatttcattcattatattcaggtagtccatggacattaattcaacatcgaatagatggatcacaaaacttcaatgaaacgtgggagaactacaaatatggttttgggaggcttgatggtaaggggactacattcaatcattcattcacttgctaatctacaaatatttactgagaacctcttatggaccaggtattaggaaaagtagtaacgaacgagaagcagtctcagccttcatataatttattatcaaacaattacacatttgttagtaaattacacttattacaactgttattatttgaattatatttatcacaattacatgtctgttcttaaatatacttatcacaatttaattccacggcttacaatgatcataactataattattaaagacaattttgattaaatgttatgtcataagtagtaactgttacaaataagctgtgaaaagaaccactcctagcattagtcactctattctctcattaacgttttacatatcaattaattggaagttaaaaggaccaggaaactcagacatacagtatacattttaaaatttcaattatttaaatataatatatagaatgtatggcttataatgaattagttaactcaatgcaaattattctattttgattacaaatagtaaaataagcaagataaaataacagatgtttaaaatccaaaaagcacatacaaaaatccatgaatgatgtctaagtactcacttataaagtagaagacattcattattatatcaaatttttaaatgctcagtactatttgaccatttaaaaattttgtattcaaactaccagtgaaagccctacctagaaggtatactcagtgataagttttgtagctccaaatcttctaatagtgagtgtaaccccaaaataaaaggctgacaggtaagtcgagaatactcacttaattctggtaagaaagcaacccatttgtacttgtatttaccagcaatccttaaaatgaagcttcctactaactcaatagcaataagacaatagtgaatgtttaatgaaaacagtattttataaatactttaataaaaaggattgtgatgaagaacaatctatttatatttgttatttgtttttaattccaataaaaataatttttaaaattacagaaaaaagttattaagaaccatgcttttaaatttaaaatgattttttaaatttattcctgtctttttctacaaagaaagcatacattaagcaaataccaaaggccaggtttacatttgaagaaagtgacattattattactcaagtctctaggaatacttaacacatctcttgactgtatatggatgttaataaatagctgacagtaaagtttatccatataaagacttgcaaatattcctctaccaatgacgagactttaaaatatctataataatgtaacacatttcactggtgaaacatgtcttgtcatatgcattatagaaaggataatcagactttcagttatattaatatttttaacatttttgtgcacatagctatcttcaataaaattgttttaaaaggtattattttaagatacactaaaatgatcaagggattcaagactaaacaactcaattagttgcaccaataaaaaacacttaaaaaaactgtcagtgtccaacctgtacttaataactcacagatttttaaaacttttcttttcaggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggtatctttttctgataccaatactttattttcatatcttcaaagtatcttcccacattattagctattatctgcaatgacaacttttaaaaatccgaatcccaaataagcgttttctctctagacgaaaacctcttaactataatgaaagtgttcattctagttcaatcaggtattttacctctaatcttcctcagattttctattttttggtagtgtatagattatttatacagattatttaaaattgggacttatacagattatttaaaactgggatacatgcatctaaaacactgtaatatttataagaaaggaagataaacttacggggaaatacagtaacagtaactacatacgagtctgtacccattaaattgcatatctatctcctttaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttccttaaaaaaaaaaaaaactctcataggacatgtttcattttgttcctttcaggagtagtaaattagacgttttccccatataaagcttttttctaccagaaagatacttctggtagaagaagagaaaggagctctttatggttcacacgactgtctcctgtcctaactactttgcttaaagtgctcaaattccatcactactcacagttgtctaatctaagtctaatcccctttgatctctcagactaccttcccttttatctctctactacttaataataagaatatctttttttcaaacttgaccttcattttgctttcacaatactatactctccatggattatcccttatctgaatccatctttataaccctattcctttctcatatttagtactgtgggccaatggacaaccttcaatcatcttttctacactgaccctcagacattctatctgctctcacggactcctttatttaccatgaataaagttccaaaatctacatattcatcccaagtctctttccagttccccttcttacattgcctatttgccatttctcccttcaataccctatacttcactcaaattcaacataccaaaaataaaaggccaggcacggtggctcacacctgtaatcccaggactttgggaggctgaggcaggtggatcacctgaggtcaggagtctgaccagcctgaccaatatggtgaaaccccgtctctacctaaaatacaaaaattagccaggcgtggtggcatgtgcctacagtcccagctactcaagaggctgagacaggagaatcgcttgaacccaggaggcggaggttgcagtgagctgagatcacaccaatgcactgggtgacagaacaagactgactcaaaaaaaaataaataacaaattccccagccccttactgctactgctatccctttctacccacctttccctcctttatactctttcacaccatcttcctcacttctttatatccattaatatgaccagcatgttcccagtcacagaagcctggaacccggaagacatctctggcttttcactcaactttgtaaactacctcttttgtatcataagccaccaagttcaatacaatcttctcttgaaacgtctcttaatcttataagctttcttccccaaagactgtctttaacttcagtgctagattatataagt

SEQ ID NO: 2 - DNA, 2926 bp, synthetic construct
  misc_feature 1..2926: note = NA sequence human ANGPTL3 mRNA
  source 1..2926: mol_type = other DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 3 - RNA, 28 nt, synthetic construct
  misc_feature 1..28: note = target RNA sequence
  source 1..28: mol_type = other RNA
cagagccaaaatcaagatttgctatgtt

SEQ ID NO: 4 - RNA, 26 nt, synthetic construct
  misc_feature 1..26: note = target RNA sequence
  source 1..26: mol_type = other RNA
aaactcaacatatttgatcagtcttt

SEQ ID NO: 5 - RNA, 23 nt, synthetic construct
  misc_feature 1..23: note = target RNA sequence
  source 1..23: mol_type = other RNA
caagagcaccaagaactactccc

SEQ ID NO: 6 - RNA, 23 nt, synthetic construct
  misc_feature 1..23: note = target RNA sequence
  source 1..23: mol_type = other RNA
agtccatggacattaattcaaca

SEQ ID NO: 7 - RNA, 27 nt, synthetic construct
  misc_feature 1..27: note = target RNA sequence
  source 1..27: mol_type = other RNA
gttgcgattactggcaatgtccccaat

SEQ ID NO: 8 - RNA, 22 nt, synthetic construct
  misc_feature 1..22: note = sequence comprised in said First RNA sequences
  source 1..22: mol_type = other RNA
aacatagcaaatcttgattttg

SEQ ID NO: 9 - RNA, 22 nt, synthetic construct
  misc_feature 1..22: note = sequence comprised in said First RNA sequences
  source 1..22: mol_type = other RNA
acatagcaaatcttgattttgg

SEQ ID NO: 10 - RNA, 22 nt, synthetic construct
  misc_feature 1..22: note = sequence comprised in said First RNA sequences
  source 1..22: mol_type = other RNA
catagcaaatcttgattttggc

SEQ ID NO: 11 - RNA, 22 nt, synthetic construct
  misc_feature 1..22: note = sequence comprised in said First RNA sequences
  source 1..22: mol_type = other RNA
atagcaaatcttgattttggct

SEQ ID NO: 12 - RNA, 22 nt, synthetic construct
  misc_feature 1..22: note = sequence comprised in said First RNA sequences
  source 1..22: mol_type = other RNA
tagcaaatcttgattttggctc

SEQ ID NO: 13 - RNA, 22 nt, synthetic construct
  misc_feature 1..22: note = sequence comprised in said First RNA sequences
  source 1..22: mol_type = other RNA
agcaaatcttgattttggctct

SEQ ID NO: 14 - RNA, 22 nt, synthetic construct
  misc_feature 1..22: note = sequence comprised in said First RNA sequences
  source 1..22: mol_type = other RNA
gcaaatcttgattttggctctg

SEQ ID NO: 15 - RNA, 22 nt, synthetic construct
  misc_feature 1..22: note = sequence comprised in said First RNA sequences
  source 1..22: mol_type = other RNA
gactgatcaaatatgttgagtt

SEQ ID NO: 16 - RNA, 22 nt, synthetic construct
  misc_feature 1..22: note = sequence comprised in said First RNA sequences
  source 1..22: mol_type = other RNA
agactgatcaaatatgttgagt

SEQ ID NO: 17 - RNA, 22 nt, synthetic construct
  misc_feature 1..22: note = sequence comprised in said First RNA sequences
  source 1..22: mol_type = other RNA
aagactgatcaaatatgttgag

SEQ ID NO: 18 - RNA, 22 nt, synthetic construct
  misc_feature 1..22: note = sequence comprised in said First RNA sequences
  source 1..22: mol_type = other RNA
gggagtagttcttggtgctctt

SEQ ID NO: 19 - RNA, 22 nt, synthetic construct
  misc_feature 1..22: note = sequence comprised in said First RNA sequences
  source 1..22: mol_type = other RNA
ggagtagttcttggtgctcttg

SEQ ID NO: 20 - RNA, 22 nt, synthetic construct
  misc_feature 1..22: note = sequence comprised in said First RNA sequences
  source 1..22: mol_type = other RNA
tgttgaattaatgtccatggac

SEQ ID NO: 21 - RNA, 22 nt, synthetic construct
  misc_feature 1..22: note = sequence comprised in said First RNA sequences
  source 1..22: mol_type = other RNA
gttgaattaatgtccatggact

SEQ ID NO: 22 - RNA, 22 nt, synthetic construct
  misc_feature 1..22: note = sequence comprised in said First RNA sequences
  source 1..22: mol_type = other RNA
ggggacattgccagtaatcgca

SEQ ID NO: 23 - RNA, 22 nt, synthetic construct
  misc_feature 1..22: note = sequence comprised in said First RNA sequences
  source 1..22: mol_type = other RNA
gggacattgccagtaatcgcaa

SEQ ID NO: 24 - RNA, 22 nt, synthetic construct
  misc_feature 1..22: note = sequence comprised in said First RNA sequences
  source 1..22: mol_type = other RNA
ggacattgccagtaatcgcaac

SEQ ID NO: 25 - RNA, 22 nt, synthetic construct
  misc_feature 1..22: note = sequence comprised in said First RNA sequences
  source 1..22: mol_type = other RNA
attggggacattgccagtaatc

SEQ ID NO: 26 - RNA, 17 nt, synthetic construct
  misc_feature 1..17: note = second RNA sequences
  source 1..17: mol_type = other RNA
atcaagatttgctatgt

SEQ ID NO: 27 - RNA, 17 nt, synthetic construct
  misc_feature 1..17: note = second RNA sequences
  source 1..17: mol_type = other RNA
aatcaagatttgctatg

SEQ ID NO: 28 - RNA, 17 nt, synthetic construct
  misc_feature 1..17: note = second RNA sequences
  source 1..17: mol_type = other RNA
aaatcaagatttgctat

SEQ ID NO: 29 - RNA, 17 nt, synthetic construct
  misc_feature 1..17: note = second RNA sequences
  source 1..17: mol_type = other RNA
aaaatcaagatttgcta

SEQ ID NO: 30 - RNA, 17 nt, synthetic construct
  misc_feature 1..17: note = second RNA sequences
  source 1..17: mol_type = other RNA
caaaatcaagatttgct

SEQ ID NO: 31 - RNA, 17 nt, synthetic construct
  misc_feature 1..17: note = second RNA sequences
  source 1..17: mol_type = other RNA
ccaaaatcaagatttgc

SEQ ID NO: 32 - RNA, 17 nt, synthetic construct
  misc_feature 1..17: note = second RNA sequences
  source 1..17: mol_type = other RNA
gccaaaatcaagatttg

SEQ ID NO: 33 - RNA, 17 nt, synthetic construct
  misc_feature 1..17: note = second RNA sequences
  source 1..17: mol_type = other RNA
caacatatttgatcagt

SEQ ID NO: 34 - RNA, 17 nt, synthetic construct
  misc_feature 1..17: note = second RNA sequences
  source 1..17: mol_type = other RNA
aacatatttgatcagtc

SEQ ID NO: 35 - RNA, 17 nt, synthetic construct
  misc_feature 1..17: note = second RNA sequences
  source 1..17: mol_type = other RNA
acatatttgatcagtct

SEQ ID NO: 36 - RNA, 17 nt, synthetic construct
  misc_feature 1..17: note = second RNA sequences
  source 1..17: mol_type = other RNA
gcaccaagaactactcc

SEQ ID NO: 37 - RNA, 17 nt, synthetic construct
  misc_feature 1..17: note = second RNA sequences
  source 1..17: mol_type = other RNA
agcaccaagaactactc

SEQ ID NO: 38 - RNA, 17 nt, synthetic construct
  misc_feature 1..17: note = second RNA sequences
  source 1..17: mol_type = other RNA
atggacattaattcaac

SEQ ID NO: 39 - RNA, 17 nt, synthetic construct
  misc_feature 1..17: note = second RNA sequences
  source 1..17: mol_type = other RNA
catggacattaattcaa

SEQ ID NO: 40 - RNA, 17 nt, synthetic construct
  misc_feature 1..17: note = second RNA sequences
  source 1..17: mol_type = other RNA
attactggcaatgtccc

SEQ ID NO: 41 - RNA, 17 nt, synthetic construct
  misc_feature 1..17: note = second RNA sequences
  source 1..17: mol_type = other RNA
gattactggcaatgtcc

SEQ ID NO: 42 - RNA, 17 nt, synthetic construct
  misc_feature 1..17: note = second RNA sequences
  source 1..17: mol_type = other RNA
cgattactggcaatgtc

SEQ ID NO: 43 - RNA, 17 nt, synthetic construct
  misc_feature 1..17: note = second RNA sequences
  source 1..17: mol_type = other RNA
actggcaatgtccccaa

SEQ ID NO: 44 - RNA, 39 nt, synthetic construct
  misc_feature 1..39: note = Combination of first and second RNA sequences
  source 1..39: mol_type = other RNA
aacatagcaaatcttgattttgatcaagatttgctatgt

SEQ ID NO: 45 - RNA, 39 nt, synthetic construct
  misc_feature 1..39: note = Combination of first and second RNA sequences
  source 1..39: mol_type = other RNA
acatagcaaatcttgattttggaatcaagatttgctatg

SEQ ID NO: 46 - RNA, 39 nt, synthetic construct
  misc_feature 1..39: note = Combination of first and second RNA sequences
  source 1..39: mol_type = other RNA
catagcaaatcttgattttggcaaatcaagatttgctat

SEQ ID NO: 47 - RNA, 39 nt, synthetic construct
  misc_feature 1..39: note = Combination of first and second RNA sequences
  source 1..39: mol_type = other RNA
atagcaaatcttgattttggctaaaatcaagatttgcta

SEQ ID NO: 48 - RNA, 39 nt, synthetic construct
  misc_feature 1..39: note = Combination of first and second RNA sequences
  source 1..39: mol_type = other RNA
tagcaaatcttgattttggctccaaaatcaagatttgct

SEQ ID NO: 49 - RNA, 39 nt, synthetic construct
  misc_feature 1..39: note = Combination of first and second RNA sequences
  source 1..39: mol_type = other RNA
agcaaatcttgattttggctctccaaaatcaagatttgc

SEQ ID NO: 50 - RNA, 39 nt, synthetic construct
  misc_feature 1..39: note = Combination of first and second RNA sequences
  source 1..39: mol_type = other RNA
gcaaatcttgattttggctctggccaaaatcaagatttg

SEQ ID NO: 51 - RNA, 39 nt, synthetic construct
  misc_feature 1..39: note = Combination of first and second RNA sequences
  source 1..39: mol_type = other RNA
gactgatcaaatatgttgagttcaacatatttgatcagt

SEQ ID NO: 52 - RNA, 39 nt, synthetic construct
  misc_feature 1..39: note = Combination of first and second RNA sequences
  source 1..39: mol_type = other RNA
agactgatcaaatatgttgagtaacatatttgatcagtc

SEQ ID NO: 53 - RNA, 39 nt, synthetic construct
  misc_feature 1..39: note = Combination of first and second RNA sequences
  source 1..39: mol_type = other RNA
aagactgatcaaatatgttgagacatatttgatcagtct

SEQ ID NO: 54 - RNA, 39 nt, synthetic construct
  misc_feature 1..39: note = Combination of first and second RNA sequences
  source 1..39: mol_type = other RNA
gggagtagttcttggtgctcttgcaccaagaactactcc

SEQ ID NO: 55 - RNA, 39 nt, synthetic construct
  misc_feature 1..39: note = Combination of first and second RNA sequences
  source 1..39: mol_type = other RNA
ggagtagttcttggtgctcttgagcaccaagaactactc

SEQ ID NO: 56 - RNA, 39 nt, synthetic construct
  misc_feature 1..39: note = Combination of first and second RNA sequences
  source 1..39: mol_type = other RNA
tgttgaattaatgtccatggacatggacattaattcaac

SEQ ID NO: 57 - RNA, 39 nt, synthetic construct
  misc_feature 1..39: note = Combination of first and second RNA sequences
  source 1..39: mol_type = other RNA
gttgaattaatgtccatggactcatggacattaattcaa

SEQ ID NO: 58 - RNA, 39 nt, synthetic construct
  misc_feature 1..39: note = Combination of first and second RNA sequences
  source 1..39: mol_type = other RNA
ggggacattgccagtaatcgcaattactggcaatgtccc

SEQ ID NO: 59 - RNA, 39 nt, synthetic construct
  misc_feature 1..39: note = Combination of first and second RNA sequences
  source 1..39: mol_type = other RNA
gggacattgccagtaatcgcaagattactggcaatgtcc

SEQ ID NO: 60 - RNA, 39 nt, synthetic construct
  misc_feature 1..39: note = Combination of first and second RNA sequences
  source 1..39: mol_type = other RNA
ggacattgccagtaatcgcaaccgattactggcaatgtc

SEQ ID NO: 61 - RNA, 39 nt, synthetic construct
  misc_feature 1..39: note = Combination of first and second RNA sequences
  source 1..39: mol_type = other RNA
attggggacattgccagtaatcactggcaatgtccccaa

SEQ ID NO: 62 - RNA, 72 nt, synthetic construct
  misc_feature 1..72: note = pri-miRNA sequences
  source 1..72: mol_type = other RNA
cttgggaatggcaaggaacatagcaaatcttgattttgatcaagatttgctatgtctcttgctatacccaga

SEQ ID NO: 63 - RNA, 72 nt, synthetic construct
  misc_feature 1..72: note = pri-miRNA sequences
  source 1..72: mol_type = other RNA
cttgggaatggcaaggacatagcaaatcttgattttggaatcaagatttgctatgctcttgctatacccaga

SEQ ID NO: 64 - RNA, 72 nt, synthetic construct
  misc_feature 1..72: note = pri-miRNA sequences
  source 1..72: mol_type = other RNA
cttgggaatggcaaggcatagcaaatcttgattttggcaaatcaagatttgctatctcttgctatacccaga

SEQ ID NO: 65 - RNA, 72 nt, synthetic construct
  misc_feature 1..72: note = pri-miRNA sequences
  source 1..72: mol_type = other RNA
cttgggaatggcaaggatagcaaatcttgattttggctaaaatcaagatttgctactcttgctatacccaga

SEQ ID NO: 66 - RNA, 72 nt, synthetic construct
  misc_feature 1..72: note = pri-miRNA sequences
  source 1..72: mol_type = other RNA
cttgggaatggcaaggtagcaaatcttgattttggctccaaaatcaagatttgctctcttgctatacccaga

SEQ ID NO: 67 - RNA, 72 nt, synthetic construct
  misc_feature 1..72: note = pri-miRNA sequences
  source 1..72: mol_type = other RNA
cttgggaatggcaaggagcaaatcttgattttggctctccaaaatcaagatttgcctcttgctatacccaga

SEQ ID NO: 68 - RNA, 72 nt, synthetic construct
  misc_feature 1..72: note = pri-miRNA sequences
  source 1..72: mol_type = other RNA
cttgggaatggcaagggcaaatcttgattttggctctggccaaaatcaagatttgatcttgctatacccaga

SEQ ID NO: 69 - RNA, 72 nt, synthetic construct
  misc_feature 1..72: note = pri-miRNA sequences
  source 1..72: mol_type = other RNA
cttgggaatggcaagggactgatcaaatatgttgagttcaacatatttgatcagtatcttgctatacccaga

SEQ ID NO: 70 - RNA, 72 nt, synthetic construct
  misc_feature 1..72: note = pri-miRNA sequences
  source 1..72: mol_type = other RNA
cttgggaatggcaaggagactgatcaaatatgttgagtaacatatttgatcagtcctcttgctatacccaga

SEQ ID NO: 71 - RNA, 72 nt, synthetic construct
  misc_feature 1..72: note = pri-miRNA sequences
  source 1..72: mol_type = other RNA
cttgggaatggcaaggaagactgatcaaatatgttgagacatatttgatcagtctctcttgctatacccaga

SEQ ID NO: 72 - RNA, 72 nt, synthetic construct
  misc_feature 1..72: note = pri-miRNA sequences
  source 1..72: mol_type = other RNA
cttgggaatggcaagggggagtagttcttggtgctcttgcaccaagaactactccatcttgctatacccaga

SEQ ID NO: 73 - RNA, 72 nt, synthetic construct
  misc_feature 1..72: note = pri-miRNA sequences
  source 1..72: mol_type = other RNA
cttgggaatggcaaggggagtagttcttggtgctcttgagcaccaagaactactcatcttgctatacccaga

SEQ ID NO: 74 - RNA, 72 nt, synthetic construct
  misc_feature 1..72: note = pri-miRNA sequences
  source 1..72: mol_type = other RNA
cttgggaatggcaaggtgttgaattaatgtccatggacatggacattaattcaacctcttgctatacccaga

SEQ ID NO: 75 - RNA, 72 nt, synthetic construct
  misc_feature 1..72: note = pri-miRNA sequences
  source 1..72: mol_type = other RNA
cttgggaatggcaagggttgaattaatgtccatggactcatggacattaattcaaatcttgctatacccaga

SEQ ID NO: 76 - RNA, 72 nt, synthetic construct
  misc_feature 1..72: note = pri-miRNA sequences
  source 1..72: mol_type = other RNA
cttgggaatggcaaggggggacattgccagtaatcgcaattactggcaatgtcccatcttgctatacccaga

SEQ ID NO: 77 - RNA, 72 nt, synthetic construct
  misc_feature 1..72: note = pri-miRNA sequences
  source 1..72: mol_type = other RNA
cttgggaatggcaagggggacattgccagtaatcgcaagattactggcaatgtccatcttgctatacccaga

SEQ ID NO: 78 - RNA, 72 nt, synthetic construct
  misc_feature 1..72: note = pri-miRNA sequences
  source 1..72: mol_type = other RNA
cttgggaatggcaaggggacattgccagtaatcgcaaccgattactggcaatgtcatcttgctatacccaga

SEQ ID NO: 79 - RNA, 72 nt, synthetic construct
  misc_feature 1..72: note = pri-miRNA sequences
  source 1..72: mol_type = other RNA
cttgggaatggcaaggattggggacattgccagtaatcactggcaatgtccccaactcttgctatacccaga

SEQ ID NO: 80 - DNA, 243 bp, synthetic construct
  misc_feature 1..243: note = Q1 promoter sequence
  source 1..243: mol_type = other DNA
aagcaaatatttgtggttatggattaactcgaactgtttgcccactctatttgcccggcgccctttggaccttttgcaatcctggagcaaacagcaaacacggacttagcccctgtttgctcctccgataactggggtgaccttggttaatattcaccagcagcctcgggcatataaaacaggggcaaggcacagactcatagcagagcaatcaccaccaagcctggaataactgcagccacc

SEQ ID NO: 81 - DNA, 278 bp, synthetic construct
  misc_feature 1..278: note = Q1-prime promoter sequence
  source 1..278: mol_type = other DNA
aagcaaatatttgtggttatggattaactcgaactgtttgcccactctatttgcccggcgccctttggaccttttgcaatcctggagcaaacagcaaacacgactcagatcccagccagtggacttagcccctgtttgctcctccgataactggggtgaccttggttaatattcaccagcagcctcccccgttgcccctctggggcatataaaacaggggcaaggcacagactcatagcagagcaatcaccaccaagcctggaataactgcagccacc

SEQ ID NO: 82 - DNA, 193 bp, synthetic construct
  misc_feature 1..193: note = C14 promoter sequence
  source 1..193: mol_type = other DNA
ttaatatttaacatcctagcacagcttcacttccaggtatgacctttgaacctcttctagaagggtaattattaacctagctaggtatgaccttcgaacctcttctagaagtgaagctgggcatataaacaggggcaaggcacagactcatagcagagcaattaccaccaagcctggaatagctgcagccacc

SEQ ID NO: 83 - DNA, 222 bp, synthetic construct
  misc_feature 1..222: note = C16 promoter sequence
  source 1..222: mol_type = other DNA
ggttaataattacccttctaggattgagtcacttctagaagctggactttggactcatcctagaagtcacttcctcttttttacctagaagaggttcaaaggtcatacctagcatagcttcacttctagaagggtaattattaaccgggcatataaaacaggggcaaggcacagactcatagcagagcaatcaccaccaggcctggaataactgcagccacc

SEQ ID NO: 84 - DNA, 238 bp, synthetic construct
  misc_feature 1..238: note = promoter sequence
  source 1..238: mol_type = other DNA
aagcaaatatttgtggttatggattaactcgaactgtttgcccactctatttgcccggcgccctttggaccttttgcaatcctggagcaaacagcaaacacggacttagcccctgtttgctcctccgataactggggtgaccttggttaatattcaccagcagcctcatgagcggaagtgggtctcaaccactataaatcctctctgtgcccgtccggagctggtgaggacagccacc

SEQ ID NO: 85 - DNA, 211 bp, synthetic construct
  misc_feature 1..211: note = promoter sequence
  source 1..211: mol_type = other DNA
taaagcaaatatttgtggttatggattaactcgaacttctagaagctgtttgcccactctatttgcccatcctaggtaggcgccctttggaccttttgcaatcctggcttctagaagagcaaacagcaaacacatcctaggtaggacttagcccctgtttgctcctccgataactggggtgaccttggttaatattcaccagcagcctcat

SEQ ID NO: 86 - DNA, 243 bp, synthetic construct; identical to SEQ ID NO: 80
  misc_feature 1..243: note = promoter sequence
  source 1..243: mol_type = other DNA

SEQ ID NO: 87 - DNA, 228 bp, synthetic construct
  misc_feature 1..228: note = promoter sequence
  source 1..228: mol_type = other DNA
aagcaaatatttgtggttatggattaactcgaactgtttgcccactctatttgcccggcgccctttggaccttttgcaatcctggagcaaacagcaaacacgcccctgtttgctcctccgataactggggtgaccttggttaatattcaccagggcatataaaacaggggcaaggcacagactcatagcagagcaatcaccaccaagcctggaataactgcagccacc

SEQ ID NO: 88 - DNA, 133 bp, synthetic construct
  misc_feature 1..133: note = SV40 polyA sequence
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 89 - DNA, 2995 bp, synthetic construct
  misc_feature 1..2995: note = Macaca fascicularis angiopoietin like 3 (ANGPTL3), mRNA
  source 1..2995: mol_type = other DNA
tacaatttcaaattacctattaagttagttgctcatttctttgatttcatttagcattgatgtaactcaatgtggaagaaggttacattcgtgcaagttaacatggcttaatgattaactatattcacctgccaaccttgccttttctgtggcaaatattggtatatatagagttaagaagtctaggtctgcttccagaagaacacagttccacgctgcttgaaattgaaaatcaggataaaaatgttcacaattaagctccttctttttattgttcctctagttatttcctccagaattgaccaagacaattcatcatttgattctgtatctccagagccaaaatcaagatttgctatgttagacgatgtaaaaattttagccaatggcctccttcagttgggacatggtcttaaagactttgtccataagactaagggccaaattaatgacatatttcaaaaactcaacatatttgatcagtctttttatgatctatcactgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgcaactccagaacatccagaagtaacttcacttaaaagttttgtagaaaaacaagataatagcatcaaagaccttctccagactgtggaagaacaatataagcaattaaaccaacagcatagtcaaataaaagaaatagaaaatcagctcagaatgactaatattcaagaacccacagaaatttctctatcttccaagccaagagcaccaagaactactccctttcttcagctgaatgaaataagaaatgtaaaacatgatggcattcctgctgattgtaccaccatttacaatagaggtgaacatataagtggcacgtatgccatcagacccagcaactctcaagtttttcatgtctactgtgatgttgtatcaggtagtccatggacattaattcaacatcgaatagatggatcacaaaacttcaatgaaacgtgggagaactacaaatatggtttcgggaggcttgatggagaattctggttgggcctagagaagatatactccatagtgaagcaatctaattacgttttacgaattgagttggaagactggaaagacaacaaacattatattgaatattctttttacttgggaaatcacgaaaccaactatacgctacatgtagttaagattactggcaatgtccccaatgcaatcccggaaaacaaagatttggtgttttctacttgggatcacaaagcaaaaggacacttcagctgtccagagagttattcaggaggctggtggtggcatgatgagtgtggagaaaacaacctaaatggtaaatataacaaaccaagaacaaaatctaagccagagcggagaagaggattatcctggaagtctcaaaatggaaggttatactctataaaatcaaccaaaatgttgatccatccaacagattcagaaagctttgaatgaactgaggcaaatttaaaaggcaataaattaaacattaaactcattccaagttaatgtggtttaataatctggtattaaatccttaagagaaggcttgagaaatagatttttttatcttaaagtcactgtcaatttaagattaaacatacaatcacataaccttaaagaataccatttacatttctcaatcaaaattcttacaacactatttgttttatattttgtgatgtgggaatcaattttagatggtcgcaatctaaattataatcaacaggtgaacttactaaataacttttctaaataaaaaacttagagactttaattttaaaagtcatcatatgagctaatatcacaattttcccagtttaaaaaactagttttcttgttaaaactctaaacttgactaaataaagaggactgataattatacagttcttaaatttgttgtaatattaatttcaaaactaaaaattgtcagcacagagtatgtgtaaaaatctgtaatataaatttttaaactgatgcctcattttgctacaaaataatctggagtaaatttttgataggatttatttatgaaacctaatgaagcaggattaaatactgtattaaaataggttcgctgtcttttaaacaaatggagatgatgattactaagtcacattgactttaatatgaggtatcactataccttaacatatttgttaaaacgtatactgtatacattttgtgtattttaatacttaatactatgaaaacaagtaattgtaaacgtatcttgtcagattacaataggaatgaacatattggtgacatcgagttaaagtttatatttcccctaaatatgctgcgattccaatatattcatgtaggttttcaagcagaaataaaccttgtaacaagttactgactaaacagcctgacaagtatgtatatatgtttaaaattcaataaataaagacccagtcttctaaattataaaaatttaaattagtcttgcacaaattaaattattcatcacaaaagatgtattgttatttttaagtcatttaagccctaaatccctaaagattagatataaattttttttgccagagtataaattgtcagaatttatttttaaatatattttttaaaactaccagtaagaaattttaaattaaacccatttgttaaaggatatagtgcccaagttatacggtgacctacctttgtcaatatttagcattatgtatttcaaattatccaatatacatgtcatatatatttttatatgttgcatatataaaagatatacacgatttatgtgaatcctatgtaaatattttgttccagaaaagtacaaaataataaaggtaaaaataatcca

SEQ ID NO: 90 - DNA, 1609 bp, synthetic construct
  misc_feature 1..1609: note = NA sequence mouse Angptl3 mRNA
  source 1..1609: mol_type = other DNA
acaggagggagaagttccaaattgcttaaaattgaataattgagacaaaaaatgcacacaattaaattattcctttttgttgttcctttagtaattgcatccagagtggatccagacctttcatcatttgattctgcaccttcagagccaaaatcaagatttgctatgttggatgatgtcaaaattttagcgaatggcctcctgcagctgggtcatggacttaaagattttgtccataagactaagggacaaattaacgacatatttcagaagctcaacatatttgatcagtctttttatgacctatcacttcgaaccaatgaaatcaaagaagaggaaaaggagctaagaagaactacatctacactacaagttaaaaacgaggaggtgaagaacatgtcagtagaactgaactcaaagcttgagagtctgctggaagagaagacagcccttcaacacaaggtcagggctttggaggagcagctaaccaacttaattctaagcccagctggggctcaggagcacccagaagtaacatcactcaaaagttttgtagaacagcaagacaacagcataagagaactcctccagagtgtggaagaacagtataaacaattaagtcaacagcacatgcagataaaagaaatagaaaagcagctcagaaagactggtattcaagaaccctcagaaaattctctttcttctaaatcaagagcaccaagaactactccccctcttcaactgaacgaaacagaaaatacagaacaagatgaccttcctgccgactgctctgccgtttataacagaggcgaacatacaagtggcgtgtacactattaaaccaagaaactcccaagggtttaatgtctactgtgatacccaatcaggcagtccatggacattaattcaacaccggaaagatggctcacaggacttcaacgaaacatgggaaaactacgaaaagggctttgggaggctcgatggagaattttggttgggcctagagaagatctatgctatagtccaacagtctaactacattttacgactcgagctacaagactggaaagacagcaagcactacgttgaatactcctttcacctgggcagtcacgaaaccaactacacgctacatgtggctgagattgctggcaatatccctggggccctcccagagcacacagacctgatgttttctacatggaatcacagagcaaagggacagctctactgtccagaaagttactcaggtggctggtggtggaatgacatatgtggagaaaacaacctaaatggaaaatacaacaaacccagaaccaaatccagaccagagagaagaagagggatctactggagacctcagagcagaaagctctatgctatcaaatcatccaaaatgatgctccagcccaccacctaagaagcttcaactgaactgagacaaaataaaagatcaataaattaaatattaaagtcctcccgatcactgtagtaatctggtattaaaattttaatggaaagcttgagaattgaatttcaattaggtttaaactcattgttaagatcagatatcaccgaatcaacgtaaacaaaatttatctttttcaatc

SEQ ID NO: 91 - DNA, 1785 bp, synthetic construct
  misc_feature 1..1785: note = NA sequence rat Angptl3 mRNA
  source 1..1785: mol_type = other DNA
gacgttccaaattgcttgaaattgaataattgaaacaaaaatgcacacaattaagctgctcctttttgttgttcctctagtaatttcgtccagagttgatccagacctttcgccatttgattctgtaccgtcagagccaaaatcaagatttgctatgttggatgatgtcaaaattttagccaatggcctcctgcagctgggtcatggtcttaaagattttgtccataagacaaagggacaaattaatgacatatttcagaagctcaacatatttgatcagtgtttttatgacctatcacttcaaaccaatgaaatcaaagaagaggaaaaggagctaagaagaaccacatctaaactacaagttaaaaacgaagaggtgaagaatatgtcacttgaactgaactcaaagcttgaaagtctactggaggagaagatggcgctccaacacagagtcagggctttggaggaacagctgaccagcttggttcagaacccgcctggggctcgggagcacccagaggtaacgtcacttaaaagttttgtagaacagcaagataacagcataagagaactcctccagagtgtggaagaacaatataaacaactaagtcaacagcacattcagataaaagaaatagaaaatcagctcagaaagactggcattcaagaacccactgaaaattctctttattctaaaccaagagcaccaagaactactccccctcttcatctgaaggaagcaaaaaatatagaacaagatgatctgcctgctgactgctctgccatttataacagaggtgaacatacaagtggcgtgtatactattagaccaagcagctctcaagtgtttaatgtctactgtgacacccaatcaggcactccacggacattaattcaacaccggaaagatggctctcaaaacttcaaccaaacgtgggaaaactacgaaaagggttttgggaggcttgatggtaaagtgatttccttgcatcactcacttatctgttgatttaatagtattagttgggtgtgttgacacaggcctgagaccatagcgcttttgggcaaggggggaggaggagcagcaggtgaattgaaagttcaagaccagtctgggccacacattgatactccttctcgacattaagaattataaattaagcagcaattataaaatgggctgtggaaatgtaacaataagcaaaagcagaccccagtcttcataaaactgattggtaaatattatccatgatagcaactgcaatgatctcattgtacttatcactactgcatgcctgcagtatgcttgttgaaacttaattctatagttcatggttatcataagtcttattaaggaacatagtatacgccattggctctagtgaggggccatgctacaaatgagctgcaaagatagcagtatagagctctttcagtgatatcctaagcacaacgtaacacaggtgaaatgggctggaggcacagttgtggtggaacacgcggccagcaggacactgggactgatccccagcagcacaaagaaagtgataggaacacagagcgagagttagaagggacagggtcaccgtcagagatacggtgtctaactcctgcaaccctacctgtaattattccatattataaacatatactatataactgtgggtctctgcatgttctagaatatgaattctatttgattgtaaaacaaaactataaaaataagtaaaaaaataaaaaataaacagatacttaaaatcaaaaaaaaaaaaaaaaaaaaaaaaa

SEQ ID NO: 92 - DNA, 22 bp, synthetic construct
  misc_feature 1..22: note = NA sequence miANG-SCR1 guide
  source 1..22: mol_type = other DNA
gtagttctattagcgcttacta

SEQ ID NO: 93 - DNA, 22 bp, synthetic construct
  misc_feature 1..22: note = NA sequence miANG-SCR2 guide
  source 1..22: mol_type = other DNA
atggatcgagtctcgttatata

SEQ ID NO: 94 - DNA, 722 bp, synthetic construct
  misc_feature 1..722: note = HCR-hAAT promoter sequence
  source 1..722: mol_type = other DNA
ggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagt

SEQ ID NO: 95 - DNA, 200 bp, synthetic construct
  misc_feature 1..200: note = NA sequence LucANG-A
  source 1..200: mol_type = other DNA
ttctctatctccagagccaaaatcaagatttgctatgttagacgatgtaagacatatttcaaaaactcaacatatttgatcagtctttttatgatctatctctatcttccaagccaagagcaccaagaactactccctttcttcagttgaatgttatatcaggtagtccatggacattaattcaacatcgaatagatgga

SEQ ID NO: 96 - DNA, 50 bp, synthetic construct
  misc_feature 1..50: note = NA sequence LucANG-B
  source 1..50: mol_type = other DNA
tacgctacatctagttgcgattactggcaatgtccccaatgcaatcccgg

SEQ ID NO: 97 - DNA, 1107 bp, synthetic construct
  misc_feature 1..1107: note = NA hAAT - pri-miANG5 cassette
  source 1..1107: mol_type = other DNA
ggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtctctggagcctgacaaggaggacaggagagatgctgcaagcccaagaagctctctgctcagcctgtcacaacctactgactgccagggcacttgggaatggcaaggtagcaaatcttgattttggctccaaaatcaagatttgctctcttgctatacccagaaaacgtgccaggaagagaactcaggaccctgaagcagactactggaagggagactccagctcaaacaaggcaggggtgggggcgtgggattgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 98 - DNA, 1519 bp, synthetic construct
  misc_feature 1..1519: note = viral vector genome of hAAT - pri-miANG5
  source 1..1519: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgccggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtctctggagcctgacaaggaggacaggagagatgctgcaagcccaagaagctctctgctcagcctgtcacaacctactgactgccagggcacttgggaatggcaaggtagcaaatcttgattttggctccaaaatcaagatttgctctcttgctatacccagaaaacgtgccaggaagagaactcaggaccctgaagcagactactggaagggagactccagctcaaacaaggcaggggtgggggcgtgggattgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 99 - protein, 725 aa, synthetic construct
  REGION 1..725: note = VP3 AAV5 construct (hybrid VP1) 30
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 100 - DNA, 18 bp, synthetic construct
  misc_feature 1..18: note = primer forward for vector genome quantification
  source 1..18: mol_type = other DNA
gggaggtgtgggaggttt

SEQ ID NO: 101 - DNA, 20 bp, synthetic construct
  misc_feature 1..20: note = primer reverse for vector genome quantification
  source 1..20: mol_type = other DNA
aatgattaacccgccatgct

SEQ ID NO: 102 - DNA, 25 bp, synthetic construct
  misc_feature 1..25: note = probe for vector genome quantification
  source 1..25: mol_type = other DNA
acttatctacagatctgcggccgct

SEQ ID NO: 103 - DNA, 24 bp, synthetic construct
  misc_feature 1..24: note = primer forward for ACTB quantification
  source 1..24: mol_type = other DNA
aggtgcacagtaggtctgaacaga

SEQ ID NO: 104 - DNA, 22 bp, synthetic construct
  misc_feature 1..22: note = primer reverse for ACTB quantification
  source 1..22: mol_type = other DNA
tgcaaagaacacggctaagtgt

SEQ ID NO: 105 - DNA, 24 bp, synthetic construct
  misc_feature 1..24: note = target sequence for reverse transcription miANG5 24 nts primer
  source 1..24: mol_type = other DNA
tagcaaatcttgattttggctcca

SEQ ID NO: 106 - DNA, 24 bp, synthetic construct
  misc_feature 1..24: note = target sequence for probe 24 nts miANG5 design
  source 1..24: mol_type = other DNA
tagcaaatcttgattttggctcca

SEQ ID NO: 107 - RNA, 48 nt, synthetic construct
  misc_feature 1..48: note = synthetic RNA oligo for miANG5 24 nts quantification
  source 1..48: mol_type = other RNA
rtrargrcrararartrcrtrtrgrartrtrtrtrgrgrcrtrcrcra

SEQ ID NO: 108 - DNA, 427 bp, synthetic construct
  misc_feature 1..427: note = promoter sequence
  source 1..427: mol_type = other DNA
taaagcaaatatttgtggttatggattaactcgaacttctagaagctgtttgcccactctatttgcccatcctaggtaggcgccctttggaccttttgcaatcctggcttctagaagagcaaacagcaaacacatcctaggtaggacttagcccctgtttgctcctccgataactggggtgaccttggttaatattcaccagcagcctcatgctagcctcgaggatatcagatctgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccacc

SEQ ID NO: 109 - DNA, 305 bp, synthetic construct
  misc_feature 1..305: note = promoter sequence
  source 1..305: mol_type = other DNA
taaagcaaatatttgtggttatggattaactcgaacttctagaagctgtttgcccactctatttgcccatcctaggtaggcgccctttggaccttttgcaatcctggcttctagaagagcaaacagcaaacacatcctaggtaggacttagcccctgtttgctcctccgataactggggtgaccttggttaatattcaccagcagcctcatgctagcctcgaggatatcagatcttcatctatttcctgcccacatctggtataaaaggaggcagtggcccacagaggagcacagctgtgccacc

SEQ ID NO: 110 - DNA, 193 bp, Synthetic construct; identical to SEQ ID NO: 82
  misc_feature 1..193: note = promoter sequence
  source 1..193: mol_type = other DNA

SEQ ID NO: 111 - DNA, 222 bp, synthetic construct; identical to SEQ ID NO: 83
  misc_feature 1..222: note = promoter sequence
  source 1..222: mol_type = other DNA

SEQ ID NO: 112 - DNA, 432 bp, synthetic construct
  misc_feature 1..432: note = promoter sequence
  source 1..432: mol_type = other DNA
catagcttcacttctagaagaggtcagggtgacctgggcctacctagctaggttaataattacccttctagaagtgactcaatcctagaagccggaagtggcatcctagaagaggttcaaaggtcatacctaggtaaaaaagaggaagtgacttctaggataaggaagtacttctagaagtacttccttatcctagcatagcttcacttctagaagaggttcaaaggtcatacctaggtatgacctttgaacctcttctanaagttaatatttaacatcctagaagggtaattattaacctagcaaggctgactacacgagcacatatcagcgcgtcgacgatatcagacctgggcatataaaacaggggcaaggcacagactcatagcagagcaatcaccaccaagcctggaataactgcagccaccatgg

SEQ ID NO: 113 - DNA, 297 bp, synthetic construct
  misc_feature 1..297: note = promoter sequence
  source 1..297: mol_type = other DNA
tttctctggcctaactggccggtaccgtcgactgtgctcggacctgtagatgctagtctagaagaggttcaaaggtcatacctaggataaggaagtacttctaggtaggcccaggtcaccctgacctcttctaggataaggaagtacttctagaagaggtcagggtgacctgggcctacctagaagtacttccttatcctaggtatgacctttgaacctcttctagactagcatctacaggtccgagcacagtcgacggtaccggccagttaggccagagaaatgttctgncacctg

SEQ ID NO: 114 - DNA, 263 bp, synthetic construct
  misc_feature 1..263: note = promoter sequence
  source 1..263: mol_type = other DNA
tagtagggcaaaggtcacttctagaagccggaagtggcatcctagaagtgactcaatcctagaagaggtcagggtgacctgggcctacctagaagtgactcaatcctaggatgttaaatattaacttctagtagcaaggctgactacacgagcacatatcaacgcgtcgacgatatcagatctgggcatataaaacaggggcaaggcacagactcatagcagagcaatcaccaccaagcctggaataactgcagccaccatgg

SEQ ID NO: 115 - DNA, 278 bp, synthetic construct; identical to SEQ ID NO: 81
  misc_feature 1..278: note = derivative of SEQ ID NO:94
  source 1..278: mol_type = other DNA

SEQ ID NO: 116 - DNA, 252 bp, synthetic construct
  misc_feature 1..252: note = nucleic acid sequence encoding a modified miR-451
  source 1..252: mol_type = other DNA
ctctggagcctgacaaggaggacaggagagatgctgcaagcccaagaagctctctgctcagcctgtcacaacctactgactgccagggcacttgggaatggcaaggtagcaaatcttgattttggctccaaaatcaagatttgctctcttgctatacccagaaaacgtgccaggaagagaactcaggaccctgaagcagactactggaagggagactccagctcaaacaaggcaggggtgggggcgtgggat

SEQ ID NO: 117 - DNA, 23 bp, synthetic construct
  misc_feature 1..23: note = target sequence for reverse transcription miANG5 23 nts variant T primer
  source 1..23: mol_type = other DNA
tagcaaatcttgattttggctct

SEQ ID NO: 118 - DNA, 23 bp, synthetic construct
  misc_feature 1..23: note = target sequence for probe miANG5 23 nts variant T design
  source 1..23: mol_type = other DNA
tagcaaatcttgattttggctct

SEQ ID NO: 119 - RNA, 46 nt, synthetic construct
  misc_feature 1..46: note = synthetic RNA oligo for miANG5 23 nts variant T quantification
  source 1..46: mol_type = other RNA
rtrargrcrararartrcrtrtrgrartrtrtrtrgrgrcrtrcrt

(19) United States
(12) Patent Application Publication
(10) Pub. No.: US 2023/0265434 A1
(43) Pub. Date: Aug. 24, 2023

(54) GENE CONSTRUCTS FOR SILENCING ANGIOPOIETIN-LIKE 3 (ANGPTL3) AND USES THEREOF

(71) Applicant: uniQure IP B.V., Amsterdam (NL)

(72) Inventors: Sander Jan Hendrik VAN DEVENTER, Amsterdam (NL); Ying Pui LIU, Amsterdam (NL); Vanessa ZANCANELLA, Amsterdam (NL)

(21) Appl. No.: 17/961,097

(22) Filed: Oct. 6, 2022

Related U.S. Application Data
(63) Continuation of application No. PCT/EP21/59054, filed on Apr. 7, 2021.

(30) Foreign Application Priority Data
Apr. 7, 2020 (EP) 20168507.0

(51) Int. Cl. C12N 15/113; C12N 15/86; A61P 9/10

(52) U.S. Cl. CPC C12N 15/1136; C12N 15/86; A61P 9/10; C12N 2310/531; C12N 2310/14; C12N 2750/14141

(57) ABSTRACT

The present invention relates to an RNA molecule for knocking down the expression of the Angiopoietin-like 3 (ANGPTL3) gene, to a composition comprising the RNA molecule, to the medical use of the composition, and to the treatment of dyslipidemia.

### CROSS-REFERENCE TO RELATED PATENT APPLICATIONS

This application is a continuation of International Application No. PCT/EP2021/059054 filed on Apr. 7, 2021, which claims priority to European Application No. 20168507.0, filed on Apr. 7, 2020. The specification, drawings, claims and abstract of the prior applications are incorporated herein by reference in their entireties.

### SEQUENCE LISTING

The present application is filed along with a Sequence Listing in electronic format. The Sequence Listing is provided as a file entitled 069818-0945.xml, created on Apr. 4, 2023, which is 186,985 bytes in size. The information in the electronic format of the Sequence Listing is incorporated herein by reference in its entirety.

### TECHNICAL FIELD

The present invention relates to an RNA molecule for knocking down the expression of the Angiopoietin-like 3 (ANGPTL3) gene, to a composition comprising the RNA molecule, to the medical use of the composition, and to the treatment and/or prevention of Dyslipidemia.

### BACKGROUND OF THE INVENTION

Dyslipidemia is a lipid and/or lipoprotein metabolic disorder. In patients with dyslipidemia, the levels of total cholesterol (TC), low-density lipoprotein cholesterol (LDL-C) and triglyceride (TG) concentrations are increased, and the level of high-density lipoprotein cholesterol (HDL-C) is decreased. The increased level of TG-rich lipoproteins can cause acute pancreatitis, and the increased levels of LDL-C, remnant lipoproteins (i.e. very low-density lipoprotein cholesterol (VLDL-C), intermediate-density lipoprotein cholesterol (IDL-C)), and lipoprotein (Lp) (a) can cause atherosclerosis (Nordestgaard, B. G. et. al., 2018; Rojas, M. P. et. al., 2018). Thus, dyslipidemia was found to be associated with other diseases, such as atherosclerotic cardiovascular diseases which are an indicator for the initiation of the treatment and/or prevention of dyslipidemia therapies.

Statins are a class of drugs known for treating dyslipidemia patients and patients with coronary heart diseases. Statins can decrease the LDL-C levels and can be used for treating patients with stable coronary heart diseases risk (Ling, H. et al., 2015; Toth, P. P. et al., 2018). However, there are a few problems with statins. It has been found that statins cannot reduce the highly elevated TG levels (Toth, P. P. et. al., 2018). Further, some patients have low tolerance to statins. Also, statins are not suitable for patients with heart failure or end-stage renal disease (Ling, H. et. al., 2015). Other drugs, such as ezetimibe or protein convertase subtilisin/kexin type 9 (PCSK9) inhibitors, can decrease the lipid level, but the use of one of those drugs alone does not decrease the risk for atherosclerotic disease. Hence, drugs presently known for treating dyslipidemia do not meet the needs of medical practitioners and/or patients.

A number of proteins, such as Angiopoietin-like 3 protein (ANGPTL3), apolipoprotein C-III (ApoC-III), cholesterol ester transfer protein (CETP), and Lp(a) were identified to be connected with dyslipidemia, and their roles in the cholesterol or TG metabolism were studied. For instance, the transcript of the ANGPTL3 gene can inhibit the activities of lipoprotein lipase (LPL) and thus the hydrolysis of TGs in capillaries of adipose tissue and muscles, and endothelial lipase (EL) with an effect on serum HDL-C levels (Olkkonen, V. M. et. al., 2018). Persons who are homozygous or compound heterozygous for null variants in ANGPTL3 gene (NCBI Reference Sequence: NG 028169.1; SEQ ID NO.1) have the levels of plasma LDL-C and TG which are approximately 70% lower than those in persons without such variants. Also, those homozygous or compound heterozygous for null variants in ANGPTL3 gene have an enhanced insulin sensitivity without an increased prevalence of fatty liver disease or an apparent increased risk of cardiovascular disease (Graham, M. J. et al., 2017). It was also found that loss-of-function mutations of the ANGPTL3 gene occur naturally without disease symptoms and thereby the mutations are considered safe. Individuals with low ANGPTL3 protein level showed no adverse effects on the whole-body cholesterol homeostasis and no pathological conditions (Minicocci, I., et. al., 2012).

An FDA-approved drug, ARO-ANG3, was developed based on the manipulation of the small interfering RNA (siRNA), which shows the effect of reducing ANGPTL3 expression in liver and serum TG and LDL-C in multiple pre-clinical dyslipidemic small and large animal models. Another drug, called ARO-APOC3, was also designed based on the manipulation of siRNA for the treatment of severe hypertriglyceridemia (HTG) and familial chylomicronemia syndrome.

However, those drugs require frequent injections into the human body, and thereby the use of those drugs is less convenient for medical practitioners and/or patients. Moreover, such administration dosage regimes can incur high medical costs. Hence, despite their effects, those drugs remain less satisfying for medical practitioners and/or patients.

Based on the above, there is a need to have a drug for treating dyslipidemia, which meets all the above-mentioned needs.

### SUMMARY OF THE INVENTION

According to the present invention, a nucleic acid is provided. Said nucleic acid comprises a nucleic acid sequence encoding an RNA molecule which comprises a first RNA sequence and a second RNA sequence, wherein said first RNA sequence is substantially complementary to said second RNA sequence, wherein said first RNA sequence comprises a sequence that is substantially complementary to a target RNA sequence comprised in an RNA encoded by an Angiopoietin-like 3 (ANGPTL3) gene, wherein said sequence substantially complementary to said target RNA sequence has at least 19 nucleotides. Said RNA molecule, as described above, includes a double-stranded RNA (dsRNA), small interfering RNA (siRNA), microRNA (miRNA), short hairpin RNA (shRNA) or an RNA hairpin, wherein the first sequence of said dsRNA, said siRNA, said miRNA, said shRNA or said RNA hairpin comprises a sequence substantially complementary to a target sequence.

Said sequence comprised in said first RNA sequence, as described above, is substantially complementary to the target RNA sequence, and thereby said RNA molecule, as described above, has a binding specificity to the target RNA sequence. After said sequence comprised in said first RNA sequence is loaded into the RNAi Induced Silencing Complex (RISC) and binds to the target RNA sequence encoded by the ANGPTL3 gene, the transcripts of the ANGPTL3 gene are subsequently cleaved, and thereby said transcripts are decreased and/or knocked down. Suitably, said transcripts of the ANGPTL3 gene are ANGPTL3 mRNA. As a result, the activities of LPL and EL in the human body remain without being inhibited, and thereby the levels LDL-C, TC, and/or TG are decreased. Also, the risks of atherosclerosis cardiovascular diseases are lowered.

By the use of said nucleic acid, as described above and herein, the cholesterol levels in the plasma, phospholipids levels, TC, LDL-C, and/or TG levels are reduced and/or inhibited.

Furthermore, said nucleic acid, as described above, is safe to be administered into the liver.

The safety of said nucleic acid, as described above, is evaluated by measuring the alanine transaminase (ALT) activity level in the plasma and/or the Aspartate transaminase (AST) activity level in the plasma, preferably measuring both. When the liver is damaged, the AST originally present in the liver is released into the blood, and thereby the AST level in the plasma is increased. Hence, the increase of AST activity level in the plasma is an indicator of liver damage. Similar to the AST, the increased ALT level is also an indicator of liver damage.

The use of said nucleic acid, as described above does not result in a permanent increase of the AST and ALT activity levels. Hence, it is safe to administer said nucleic acid into mammals.

Moreover, the lesions of atherosclerosis are inhibited and/or reduced by using said nucleic acid. Thereby, said nucleic acid is useful in treating and/or preventing initial lesions which are also known as fatty streaks (type I-II), mild lesions (type-III), and/or severe lesions which are also known as (fibro)atheroma lesions (type IV-V). For determining atherosclerotic lesion size and severity, the lesions were classified into five categories according to the American Heart Association (Stary, H. C. et al., 1995; Stary, H. C., 2000): type I as described above and herein is early fatty streak; type II as described above and herein is regular fatty streak; type III as described above and herein is mild plaque; type IV as described above is moderate plaque; type V as described above and herein is severe plaque.

Preferably, said first RNA sequence comprised in said RNA molecule, as described above, is substantially complementary to said second RNA sequence.

Preferably, said first RNA sequence comprised in said RNA molecule, as described above, is complementary to said second RNA sequence, and thereby said first RNA sequences binds to said second RNA sequence.

Said nucleic acid, as described above, is delivered into a target cell, by for example, an adeno-associated virus (AAV) vehicle, as described herein and below. Said nucleic acid subsequently is transcribed into an RNA molecule, as described above. In the nucleus of said target cell, said RNA molecule, as described above, is cleaved by Drosha (i.e. a class 2 ribonuclease III enzyme) into a shRNA and/or an RNA hairpin without the flanking regions at the 5′ and 3′ ends of the RNA molecule. Subsequently, the cleaved RNA molecule is exported to the cytoplasm of the cell, wherein said cleaved RNA molecule is not further cleaved by an endoribonuclease Dicer, but the said cleaved RNA molecule is further cleaved by Argonaute-2 (AGO-2) of the RNA-induced silencing complex (RISC), in particular that the second RNA sequence of said cleaved RNA molecule is trimmed off (that is, degraded) from said cleaved RNA molecule, as described above. Hence, the “off-target” issue resulting from partial complementarity of said second RNA sequence of said RNA molecule to an off-target mRNA and from binding to said off-target mRNA is reduced and/or inhibited. Said second RNA sequence is also called as a passenger strand. Thereby, the binding of said first RNA sequence, also known as a guide strand, to said target RNA sequence is improved.

Suitably, a sequence complementary to said first RNA sequence, as described above, comprises at least 5, 6, 7, 8, 9, 10, or 11 nucleotides different from said second RNA sequence, as described above.

Suitably, a sequence complementary to said first RNA sequence, as described above, comprises at most 12, 13, 14, 15, or 16 nucleotides different from said second RNA sequence, as described above.

Said first and said second RNA sequences can be not complementary in multiple nucleotides, as described above, whereas said first RNA sequence remains to have enough binding specificity to said second RNA sequence. Therefore, said first and second RNA sequences, as described above, can be used in meeting the needs, as described above, such as inhibiting and/or reducing said “off-target” issue. Preferably, the RNA molecule, as described above, encoded by said nucleic acid, as described above, has a secondary structure, and/or includes a double-stranded RNA (dsRNA), small interfering RNA (siRNA), microRNA (miRNA), or an RNA hairpin, wherein the first sequence of said dsRNA or said RNA hairpin comprises a sequence substantially complementary to a target sequence.

Said nucleic acid, as described above, can be transcribed into said RNA molecule, as described above. Said RNA molecule, as described above, is useful in the present invention for inhibiting and/or further reducing said “off-target” issue. Also preferably, said RNA hairpin, as described above, is a short hairpin RNA (shRNA) or a long hairpin RNA (lhRNA). More preferably, the RNA molecule, as described above, comprises a miR-451. Still preferably, said RNA molecule is encoded from SEQ ID NO. 124, which is a nucleic acid sequence encoding modified miR-451. A nucleic acid sequence such as SEQ ID NO. 124 or the sequence encoding said miR-451, as described above, is suitable to be comprised in a vector comprised in a gene therapy vehicle such as an AAV gene therapy vehicle, and to be delivered into a target organ. Moreover, said “off-target” issue by using said RNA molecule encoded by said nucleic acid sequences is reduced and/or inhibited. Thereby, said nucleic acid satisfies the needs, as described above.

Said sequence comprised in the first RNA sequence, as described above, has optionally at least 15 nucleotides, has optionally at least 16 nucleotides, has optionally at least 17 nucleotides, has optionally at least 18 nucleotides, has optionally at least 19 nucleotides, optionally at least 20 nucleotides, optionally at least 21 nucleotides, optionally at least 22 nucleotides, or optionally at least 24 nucleotides. Further, said sequence comprised in said first RNA sequence, as described above, has optionally at most 30 nucleotides, optionally at most 28 nucleotides, or optionally at most 26 nucleotides. Said first RNA sequences comprising different nucleotides, as described above, have enough binding specificity to said target RNA sequence, as described above, and thereby said first RNA sequences are useful in reducing and/or knocking down the transcripts of the ANGPTL3 gene. Moreover, a nucleic acid comprising a nucleic acid sequence encoding one of said first RNA sequences having different lengths, as described above, can be suitably and/or easily embedded in a vector comprised in a gene therapy vehicle, and can further be folded into said RNA secondary structure, as described above. Thereby, said first RNA sequences comprising different nucleotides, as described above, are suitable to be used to target, bind to, cleave, and/or knock down the transcripts of ANGPTL3 gene, as described above, and also satisfy the needs, as described above.

Said sequence comprised in the first RNA sequence, as described above, is designed based on the conserved sequences comprised in the ANGPTL3 gene, as described below.

Preferably, said conserved sequences are mammalian conserved sequences, said mammalian conserved sequences preferably selected from rodents, such as mice, non-human primates (NHP) and humans.

Said target RNA sequence is comprised in a sequence encoded by the ANGPTL3 gene. Preferably, the ANGPTL3 gene, as described above and herein, is the mammalian ANGPTL3 gene, such as a mouse ANGPTL3 gene. More preferably, the ANGPTL3 gene is the non-human primate (NHP) ANGPTL3 gene. Most preferably, the ANGPTL3 gene is the human ANGPTL3 gene.

Said nucleic acid comprising a nucleic acid sequence encoding said first RNA sequence, as described above, said RNA molecule comprising said first RNA sequence can be useful in reducing and/or knocking down the transcripts of the ANGPTL3 gene in a mammal.

Preferably, said sequence comprised in the first RNA sequence, as described above, is one selected from the group consisting of SEQ ID NOs. 8-25. More preferably, said sequence comprised in the first RNA sequence, as described above, is one selected from the group consisting of SEQ ID NOs. 8-17 and 19-25. Still more preferably, said sequence comprised in the first RNA sequence is one selected from the group consisting of SEQ ID NOs. 11, 12, 16, 17, 20 and 25. Yet more preferably, said sequence comprised in the first RNA sequence is one selected from the group consisting of SEQ ID NOs. 11, 12, 17, 20 and 25. Most preferably, said sequence comprised in the first RNA sequence includes SEQ ID NO. 12.

When said nucleic acid, as described above, is loaded into the RISC complex, the nucleic acid sequences encoding said first RNA sequence, as described above, can reduce and/or knock down the transcripts of ANGPTL3 gene, such as the mRNA of ANGPTL3 gene. Thereby, the cholesterol level in the plasma, phospholipids level in plasma, atherosclerotic lesions, and/or the TG TC), and/or LDL-C levels are decreased in a mammal.

Said target sequence encoded by the ANGPTL3 gene, as described above, is designed to comprise a complete or a part of at least one conserved sequence encoded by the ANGPTL3 gene. A number of the conserved sequences of the ANGPTL3 gene are identified and selected for the present invention. Preferably, the conserved sequences are comprised in exon 1, exon 3, exon 5, or exon 6 of the ANGPTL3 gene, as described above. More preferably, said target sequence, as described above, comprises a complete or a part of the conserved sequence in exon 1, exon 5 or exon 6 of the ANGPTL3 gene, as described above. Yet more preferably, said target sequence, as described above, comprises a complete or a part of the conserved sequence in exon 1 or exon 5 of the ANGPTL3 gene, as described above.

Said first RNA sequence, as described above, can target and/or bind to said conserved sequences comprised in exon 1, exon 5 or exon 6 of the ANGPTL3 gene, as described above and below, and thereby reduce and/or knock down said transcripts of said ANGPTL3 gene. Subsequently, the transcripts of said ANGPTL3 gene are reduced and/or inhibited, so that the cholesterol levels in the plasma, phospholipids level in plasma, atherosclerotic lesions, and/or the triglyceride (TG), total cholesterol (TC), and/or low-density lipoprotein cholesterol (LDL-C) levels are decreased and/or inhibited in a mammal.

Said target sequence, as described above, is comprised in an RNA encoded by said ANGPTL3 gene. Preferably, said target sequence is comprised in an RNA encoded by at least a part of one exon comprised in said ANGPTL3 gene. Still preferably, said target sequence is comprised in an RNA encoded by at least one conserved sequence comprised in one exon, as described above, comprised in said ANGPTL3 gene. More preferably, said exon, as described above, is exon 1, exon 3, exon 5, or exon 6, comprised in the ANGPTL3 gene. Still more preferably, said exon, as described above, is exon 1, exon 5, or exon 6, comprised in the ANGPTL3 gene. Yet preferably, the at least one conserved sequence is the conserved sequence (NCBI reference sequence: NM_014495.4: position 139-166 nucleotides, hereafter referred to as SEQ ID NO.3) that is comprised in exon 1 of the ANGPTL3 gene, the conserved sequence (NCBI reference sequence: NM_014495.4: position 267-292 nucleotides, hereafter referred to as SEQ ID NO.4) that is comprised in exon 1 of the ANGPTL3 gene, the conserved sequence (NCBI reference sequence: NM_014495.4: position 706-728 nucleotides, hereafter referred to as SEQ ID NO.5) that is comprised in exon 3 of the ANGPTL3 gene, the conserved sequence (NCBI reference sequence: NM_014495.4: position 885-907 nucleotides, hereafter referred to as SEQ ID NO 6) that is comprised in exon 5, or the conserved sequence (NCBI reference sequence: NM_014495.4: position 1134-1160 nucleotides, hereafter referred to as SEQ ID NO.7) that is comprised in exon 6. It was found in vivo that by targeting said positions of the ANGPTL3 gene, as described above, with said first RNA sequence, as described above, said transcripts of said ANGPTL3 gene, as described above, were knocked down, and thereby the mRNA ofANGPTL3 gene was decreased and/or knocked down. Thereby, the cholesterol levels in the plasma, phospholipids level in plasma, atherosclerotic lesions, and/or the TG, TC, and/or LDL-C levels were decreased in a mammal.

Said atherosclerotic lesions comprise initial lesions, mild lesions, and/or severe lesions.

The atherosclerotic lesions are classified into types I-V according to the American Heart Association (Stary, H. C. et al., 1995; Stary, H. C., 2000). Said initial lesions as described above comprises type I-II. Said mild lesions as described above comprise type III. Said severe lesions as described above comprise type IV-V.

Hence, by targeting said positions as described above, said nucleic acid can be used in the treatment and/or prevention of lipid and/or lipoprotein metabolic disorders, such as hypercholesterolemia, hypertriglyceridemia, mixed hyperlipoproteinemia, and/or dyslipidemia, and nonalcoholic steatohepatitis (NASH).

According to the present invention, a composition comprising a nucleic acid encoding the RNA molecule, as described above, is provided.

Said RNA molecule, being in a second structure as described above, is useful in reducing and/or knocking down the transcripts of ANGPTL3 gene, and thereby the cholesterol levels in the plasma, phospholipids levels in plasma, atherosclerotic lesions, and/or the triglyceride (TG), total cholesterol (TC), and/or low-density lipoprotein cholesterol (LDL-C) levels are decreased and/or inhibited in a mammal.

Preferably, said composition further comprising at least one molecule that further reduces and/or inhibit plasma cholesterol levels, severe atherosclerotic lesions, and/or LDL-C levels.

The addition of said at least one molecule can further decrease and/or inhibit plasma cholesterol levels, severe atherosclerotic lesions, and/or LDL-C levels.

Furthermore, the use of the composition does not result in permanent increase of the AST and ALT activity levels in the plasma. Thereby, no liver damage is caused. Hence, it is safe to administer said composition, as described above, into mammals.

Preferably, said at least one molecule comprises at least one of the group of statins.

Preferably, said at least one statin is selected from the group consisting of Atorvastatin, Cerivastatin, Fluvastatin, Lovastatin, Mevastatin, Pitavastatin, Pravastatin, Rosuvastatin, and Simvastatin.

Said statins, as described above, can be used together with said composition as described above, for further decreasing and/or inhibiting the cholesterol levels in the plasma, LDL-C levels, and/or severe atherosclerotic lesions. Said severe atherosclerotic lesions as described above comprise type IV-V according to the American Heart Association (Stary, H. C. et al., 1995; Stary, H. C., 2000).

More preferably, said at least one molecule as described above, comprises Atorvastatin and/or Simvastatin.

The combined use of Atorvastatin and/or Simvastatin with said composition, as described above, is useful in decreasing and/or inhibiting the cholesterol levels in the plasma, severe atherosclerotic lesions, and/or LDL-C levels in a mammal.

According to the present invention, a composition, as described above, is used as a medicament. The therapeutic effects of said nucleic acid, as described above, were found by the present invention. Thereby, a composition comprising said nucleic acid can be used as a medicament.

Further according to the present invention, a composition, as described above, is used as a medicament for decreasing and/or knocking down the transcripts of the ANGPTL3 gene. Said composition comprising said nucleic acid, as described above, has therapeutic effects and can thus be used for treating diseases. As described above, said composition of the present invention, can decrease and/or knock down the transcripts of the ANGPTL3 gene. Said transcripts of the ANGPTL3 gene comprises the mRNA encoded by the ANGPTL3 gene. Thereby, said composition can be used as a medicament.

Also preferably, the composition, as described above, is used as a medicament, as described above, for decreasing and/or inhibiting plasma cholesterol levels, atherosclerotic lesions, phospholipids in the plasma, the LDL-C, and/or TC levels and/or the TG levels. By using said composition, as described above, the levels of cholesterol in the plasma, atherosclerotic lesions, phospholipids in the plasma, the LDL-C level, TC level, and/or TG level can be decreased and/or inhibited.

Said atherosclerotic lesions comprise initial lesions, mild lesions, and/or severe lesions.

The atherosclerotic lesions are classified into types I-V according to the American Heart Association (Stary, H. C. et al., 1995; Stary, H. C., 2000). Said initial lesions as described above comprises type I-II. said mild lesions as described above comprise type III. Said severe lesions as described above comprise type IV-V.

More preferably, the composition, as described above, is used as a medicament, as described above, for the treatment and/or prevention of lipid and/or lipoprotein metabolic disorders.

More preferably, the composition, as described above, is used as a medicament, as described above, for the treatment and/or prevention of hypercholesterolemia, hypertriglyceridemia, mixed hyperlipoproteinemia, Dyslipidemia, and/or nonalcoholic steatohepatitis (NASH).

Said composition, as described above, can decrease and/or inhibit the transcripts of the ANGPTL3 gene, and also can decrease and/or inhibit the plasma cholesterol levels, atherosclerotic lesions, phospholipids levels in the plasma, the LDL-C, TC and/or the TG, as described above. Thereby, said composition can also be used for treating and/or preventing lipid and/or lipoprotein metabolic disorders, such as hypercholesterolemia, hypertriglyceridemia, mixed hyperlipoproteinemia, Dyslipidemia, and/or nonalcoholic steatohepatitis (NASH).

Most preferably, the composition, as described above, is used as a medicament, as described above, for the treatment and/or prevention of Dyslipidemia. According to the present invention, a method for manufacturing the composition, as described above, is provided. Said method comprises a step of adding said nucleic as described above, or said RNA molecule as described above, into said composition.

Optionally, said composition further comprises at least one additive selected from the group consisting of an aqueous liquid, an organic solvent, a buffer and an excipient. Optionally, the aqueous liquid is water. Also optionally, said buffer is selected from a group consisting of acetate, citrate, phosphate, tris, histidine, and 4-(2-hydroxyethyl)-1-piperazineethanesulfonic acid (HEPES). Still optionally, the organic solvent is selected from a group consisting of ethanol, methanol, and dichloromethane. Still more, the excipient is a salt, sugar, cholesterol or fatty acid. Still optionally, said salt, as described above, is selected from a group consisting of sodium chloride, potassium chloride. Yet optionally, said sugar, as described above, is sucrose, mannitol, trehalose, and/or dextrane

According to the present invention, a DNA expression cassette is provided. The DNA expression cassette of the present invention comprises a nucleic acid sequence for encoding said RNA molecule, as described above, a promoter, a poly A tail. Said DNA expression cassette is flanked by two Inverted Terminal Repeats (ITRs).

Said nucleic acid comprised in said DNA expression cassette is useful in decreasing and/or knocking down the transcripts of ANGPTL3 gene. Said DNA expression cassette comprising said nucleic acid, as described above, can be comprised in a viral gene therapy vehicle, such as adeno-associated virus (AAV), and subsequently be delivered into a target organ. Thereby, said DNA expression cassette is useful for treating and/or preventing a human subject suffering from lipid and/or lipoprotein metabolic disorders, such as Dyslipidemia. Preferably, said promoter is selected from the group consisting of pol I promoter, pol II promoter, pol III promoter, a PGK promoter, CBA promoter, CAG promoter, CMV promoter, an inducible promoter, an al-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, HLP, minimal TTR promoter, FVIII promoter, hyperon enhancer, ealb-hAAT, EF1-Alpha promoter, Herpes Simplex Virus Tymidine Kinase (TK) promoter, U1-1 snRNA promoter, Apolipoprotein promoter, TRE promoter, rtTA-TRE (inducible promoter), LP1 promoter, Q1 promoter, Q1-prime promoter, C14 promoter, C16 promoter and any synthetic promoter selected from SEQ ID NOs. 84-87 and 108-109 and 112-115, and variants thereof.

Said promoter, as described above, is useful in initiating the expression of said nucleic acid comprised in said DNA expression cassette.

Preferably, said promoter, as described above, is a liver-specific promoter.

More preferably, said liver-specific promoter, as described above, is selected from the group consisting of an al-anti-trypsin promoter, a thyroid hormone-binding globulin promoter, an albumin promoter, LPS (thyroxine-binding globin) promoter, HCR-ApoCII hybrid promoter, HCR-20 hAAT hybrid promoter, an apolipoprotein E promoter, LP1, HLP, minimal TTR promoter, FVIII promoter, ealb-hAAT, Herpes Simplex Virus Tymidine Kinase (TK) promoter, Apolipoprotein promoter, tetracycline responsive element (TRE) promoter, LP1 promoter, Q1 promoter, Q1-prime promoter, C14 promoter, C16 promoter, and any synthetic promoter selected from SEQ ID NOs. 84-87 and 108-109 and 112-115, and variants thereof.

With the use of said liver-specific promoter in said DNA expression cassette, the expression of said nucleic acid in the liver is induced, which is useful for knocking down said transcripts of ANGPTL3 gene because said transcripts of ANGPTL3 gene are expressed predominantly in the liver.

Even more preferably, said promoter, as described above, comprises said Q1-prime promoter.

Said Q1-prime promoter is a liver-specific promoter, which can further enhance the expression of said nucleic acid, as described above, in the liver.

Optionally, each of said variants of SEQ ID NOs 84-87 and 108-109 and 112-115, as described above, has a nucleic acid sequence essentially identical to SEQ ID NOs 84-87 and 108-109 and 112-115, respectively, and said variants have substantially the same function as SEQ ID NOs 84-87 and 108-109 and 112-115 of initiating the transcription of a nucleic acid sequence encoding said RNA molecule, as described above.

Optionally, each of said variants of SEQ ID NOs 84-87 and 108-109 and 112-115, as described above, has a nucleic acid sequence comprising at least 1, 2, 3, 4, or 5 nucleotides different from the sequences of SEQ ID NOs 84-87 and 108-109 and 112-115.

Optionally, each of said variants of SEQ ID NOs 84-87 and 108-109 and 112-115, as described above, has a nucleic acid sequence comprising at most 40, 35, 30, 25, or 20 nucleotides different from the sequence of SEQ ID NOs 84-87 and 108-109 and 112-115.

Preferably, said Poly A tail comprised in said DNA expression cassette, as described above, operably links to the 3′ end of said RNA molecule, as described above. Preferably, said poly A tail is the simian virus 40 polyadenylation (SV40 polyA), synthetic polyadenylation, Bovine Growth Hormone polyadenylation (BGH polyA).

Said ITRs flanking said DNA expression cassette, as described above, are operably linked to said promoter, as described above, and said poly A tail, as described above. Preferably, said ITRs are selected from a group consisting of adeno-associated virus (AAV) ITR sequences. More preferably, said ITRs sequences comprises the AAV1, AAV2, AAV5, AAV6, or AAV8 ITRs sequences. Optionally, said two ITRs sequences comprises both AAV1, both AAV2, both AAV5, both AAV6, or both AAV8 ITRs sequences. Also optionally, said ITR sequence at the 5′ end of said DNA expression cassette differs from said ITR sequence at the 3′ of said DNA expression cassette, wherein said ITR sequence is one selected from the AAV1, AAV2, AAV5, AAV6 or AAV8 ITRs sequences. According to the present invention, a virus vehicle comprising said DNA expression cassette comprising said nucleic acid sequence encoding said RNA molecule, as described above, is provided.

Said nucleic acid, as described above, can be comprised in said DNA expression cassette which is comprised in said virus vehicle, and thereby be delivered to a target organ, such as the liver. With the use of said virus vehicle, the frequency of injecting a human subject with a therapeutic moiety is minimized, because repeated dosing is minimized. Thereby, the immune response can be reduced and/or inhibited, and/or the quality of life of said human subject is further improved.

Optionally, said virus vehicle, as described above, comprises alphavirus, flavivirus, herpes simplex viruses (HSV), measles viruses, rhabdoviruses, retrovirus, Newcastle disease virus (NDV), poxviruses, picornavirus, lentivirus, adenoviral vectors or adeno-associated virus (AAV).

Preferably, the nucleic acid for encoding the RNA molecule, as described above, is comprised in said DNA expression cassette comprised in said AAV gene therapy vehicle, as described above.

It was found that AAV is a useful gene therapy vehicle for delivery of said nucleic acid or said DNA expression cassette, as described above, into a mammal. AAV has the ability to efficiently infect dividing as well as non-dividing human cells. Moreover, AAV has not been associated with any diseases.

Still preferably, the DNA expression cassette, as described above, is comprised in said AAV gene therapy vehicle, as described above.

Said nucleic acid or said DNA expression cassette, as described above, can be comprised in said AAV gene therapy vehicle, and be subsequently delivered to a target organ. By using said AAV gene therapy vehicle, said nucleic acid or said DNA expression cassette as described above can be introduced into a human subject with a minimal risk of immune responses, and/or without repeated injections during a course of treatment.

Preferably, the capsid of said AAV gene therapy vehicle, as described above, comprises an AAV5 capsid protein sequence. Still preferably, the capsid of said AAV gene therapy vehicle, as described above, comprises an AAV2 capsid protein sequence. Yet preferably, the capsid of the said AAV gene therapy vehicle, as described above, comprises an AAV8 capsid protein sequence.

The AAV gene therapy vehicle comprising said capsid protein sequence, as described above, is suitable to be used in the present invention. Specifically, said AAV gene therapy vehicle comprising an AAV5 capsid protein sequence is useful for the present invention because the prevalence of anti-AAV5 neutralizing antibodies (Nabs) is lower than that of other serotypes. In addition, pre-existing antibodies (Abs) or low pre-existing antibodies against AAV5 does not affect transduction of said AAV gene therapy vehicle, and/or expression of said nucleic acid in a target organ. Further, no cytotoxic T-cell responses against AAV5 have been found in clinical trials. Optionally, the capsid of said AAV gene therapy vehicle, as described above, comprises an AAV5/AAV2 hybrid capsid protein sequence. Optionally, the capsid of said AAV gene therapy vehicle, as described above, comprises an AAV5/AAV8 hybrid capsid protein sequence.

Said AAV gene therapy vehicle comprising said hybrid capsid protein sequence, as described, can be useful in enhancing transduction efficacy of said AAV gene therapy vehicle to a target organ, and/or in improving targeting and/or binding to said target organ.

According to the present invention, a composition comprising said AAV gene therapy vehicle, as described above, is provided.

In said composition, as described above, said AAV gene therapy vehicle comprised in said composition comprises said DNA expression cassette, as described above. Said DNA expression cassette is flanked by said ITR sequences, as described above Said AAV gene therapy vehicle, comprises an AAV5 capsid protein or an AAV5/AAV2 hybrid capsid protein. Moreover, said DNA expression cassette comprises a sequence encoding a first RNA sequence. Preferably, the sequence comprised in the first RNA sequence is one selected from the group consisting of SEQ ID NOs. 11, 12, 16, 17, 20 and 25. Yet more preferably, said sequence comprised in the first RNA sequence is one selected from the group consisting of SEQ ID NOs. 11, 12, 17, 20 and 25. Most preferably, said sequence comprised in the first RNA sequence includes SEQ ID NO. 12. Still most preferably, said sequence comprised in the first RNA sequence, as described above, consists SEQ ID NO. 12.

Said vehicle is useful in delivering said nucleic acid sequence encoding said RNA molecule or said DNA expression cassette to a target organ, and thereby allowing said RNA molecule or said DNA expression cassette to be stably expressed in said target organ.

Said vehicles, as described herein, are used to transfer said DNA expression cassette to a target organ such that expression of said RNA molecule described above that inhibits and/or knock down of transcripts of the ANGPTL3 gene, as described above, can be achieved.

Suitable methods of production of AAV gene therapy vehicles comprising such DNA expression cassette, as described above, are described in WO2007/046703, WO2007/148971, WO2009/014445, WO2009/104964, WO2011/122950, WO2013/036118, which are incorporated herein in its entirety.

It was found in vivo that said composition, as described above, can decrease and/or knock down the transcripts of the ANGPTL3 gene, and thereby can decrease and/or inhibit the plasma cholesterol levels, atherosclerotic lesions, phospholipids in the plasma, the LDL-C, TC and/or the TG levels, as described above. Thereby, said composition can also be used for treating and/or preventing lipid and/or lipoprotein metabolic disorders, such as hypercholesterolemia, hypertriglyceridemia, mixed hyperlipoproteinemia, Dyslipidemia, and/or nonalcoholic steatohepatitis (NASH).

Said atherosclerotic lesions comprise initial lesions, mild lesions, and/or severe lesions.

The atherosclerotic lesions are classified into types I-V according to the American Heart Association (Stary, H. C. et al., 1995; Stary, H. C., 2000). Said initial lesions as described above comprises type I-II. said mild lesions as described above comprise type III. Said severe lesions as described above comprise type IV-V.

Preferably, said composition further comprising at least one molecule further reduces and/or inhibits cholesterol levels in plasma, severe atherosclerotic lesions, and/or LDL-C levels.

It was found in vivo that the addition of said at least one molecule into said composition, as described above, can further decrease severe atherosclerotic lesions, cholesterol levels in the plasma, and/or LDL-C levels.

Moreover, the addition of said at least one molecule into said composition, as described above, does not result in a permanent increase of the ALT and AST activity levels in the plasma.

Thereby, no liver damage is caused. It is therefore safe to administer said composition, as described above, into mammals.

Preferably, said at least one molecule as described above, comprises at least one of statins.

Preferably, said at least one statins, as described above, is selected from the group consisting of Atorvastatin, Cerivastatin, Fluvastatin, Lovastatin, Mevastatin, Pitavastatin, Pravastatin, Rosuvastatin, and Simvastatin.

Said statins, as described above, can be used together with said composition, as described above, for further decreasing and/or inhibiting plasma cholesterol levels, severe atherosclerotic lesions, and/or LDL-C levels.

More preferably, in said composition, as described above, said at least one molecule as described above, comprises Atorvastatin and/or Simvastatin.

It was found in vivo that compared to the use of said composition, as described, the combined use of Atorvastatin and/or Simvastatin with said composition is useful in decreasing and/or inhibiting the cholesterol levels in the plasma, severe atherosclerotic lesions, and/or LDL-C levels in a mammal.

Optionally, said composition, as described above, further comprises at least one additive selected from the group consisting of an aqueous liquid, an organic solvent, a buffer and an excipient. Optionally, the aqueous liquid is water. Also optionally, said buffer is selected from a group consisting of acetate, citrate, phosphate, tris, histidine, and 4-(2-hydroxyethyl)-1-piperazineethanesulfonic acid (HEPES). Still optionally, the organic solvent is selected from a group consisting of ethanol, methanol, and dichloromethane. Still more, the excipient is a salt, sugar, cholesterol or fatty acid. Still optionally, said salt, as described above, is selected from a group consisting of sodium chloride, potassium chloride. Yet optionally, said sugar, as described above, is sucrose, mannitol, trehalose, and/or dextrane.

According to the present invention, the use of said AAV gene therapy vehicle or said composition comprising said AAV gene therapy vehicle, as described above, as a medicament is provided.

The therapeutic effects of said AAV gene therapy vehicle or said composition comprising said AAV gene therapy vehicle, as described above, are demonstrated by the present invention. Thereby, said AAV gene therapy vehicle and said composition comprising said AAV gene therapy vehicle, as described above, can be used as a medicament.

Preferably, said medicament decreases and/or knocks down transcripts encoded by ANGPTL3 gene.

It was found in vivo that said AAV gene therapy vehicle or said composition comprising said AAV gene therapy vehicle, as described above, has the function of decreasing and/or knocking down the transcripts of the ANGPTL3 gene. Said transcripts of the ANGPTL3 gene comprises the mRNA encoded by the ANGPTL3 gene. Thereby, said AAV gene therapy vehicle can be used as a medicament.

Preferably, said medicament can be used in inhibiting and/or decreasing the cholesterol levels in the plasma, the phospholipids level, initial, mild and/or severe atherosclerosis lesions, TC level, TG level, and/or LDL-C levels.

The therapeutic effect obtained by the administration of said AAV gene therapy vehicles was shown in in vivo tests demonstrating reduced cholesterol levels in the plasma, reduced phospholipids levels, reduced initial, mild, and/or severe atherosclerosis lesions, and/or decreased levels of TC, TG and/or LDL-C levels.

Said atherosclerotic lesions comprise initial lesions, mild lesions, and/or severe lesions.

The atherosclerotic lesions are classified into types I-V according to the American Heart Association (Stary, H. C. et al., 1995; Stary, H. C., 2000). Said initial lesions as described above comprises type I-II. said mild lesions as described above comprise type III. Said severe lesions as described above comprise type IV-V.

More preferably, said medicament, as described above, is used for the treatment and/or prevention of lipid and/or lipoprotein metabolic disorders.

Still more preferably, said medicament, as described above, is used for the treatment and/or prevention of hypercholesterolemia, hypertriglyceridemia, mixed hyperlipoproteinemia, Dyslipidemia, and/or nonalcoholic steatohepatitis (NASH).

It was found that by administering said AAV gene therapy vehicle, as described above, the transcripts of ANGPTL3 gene are reduced and/or knocked down, and thereby decreased a cholesterol level in plasma, decreased a level of phospholipids, and/or decreased initial, mild and/or severe atherosclerosis lesions, and/or decreases the total cholesterol (TC) level, a level of triglyceride (TG) and/or low-density lipoprotein cholesterol (LDL-C). Therefore, said AAV gene therapy vehicle is useful in preventing and/or treating a lipid and/or lipoprotein metabolic disorder, such as Dyslipidemia.

The atherosclerotic lesions are classified into types I-V according to the American Heart Association (Stary, H. C. et al., 1995; Stary, H. C., 2000). Said initial lesions as described above comprises type I-II. said mild lesions as described above comprise type III. Said severe lesions as described above comprise type IV-V.

Most preferably, said medicament, as described above, is used for the treatment and/or prevention of Dyslipidemia.

As it was demonstrated in vivo that the transcripts of ANGPTL3 gene are reduced and/or knocked down by administering said AAV gene therapy vehicles, the AAV gene therapy vehicle, as described above, is useful in treating and/or preventing a disease in which the ANGPTL3 gene is involved. Furthermore, as also shown in in vivo experiments, cholesterol levels in plasma, phospholipids levels, atherosclerosis lesions, TC, TG, and/or LDL-C levels are reduced and/or inhibited. Therefore, said AAV gene therapy vehicles can be used as a medicament in treatment and/or preventing Dyslipidemia.

Preferably, said medicament, as described above, further comprises at least one molecule which reduces and/or inhibits the plasma cholesterol levels, level, LDL-C level, and/or severe atherosclerotic lesions.

It was found in vivo that in addition to the administration of said AAV gene therapy vehicle or said composition comprising said AAV gene therapy vehicle, as described above, the addition of said at least one compound can further enhance at least one therapeutic effect, such as further reduction and/or inhibition of the cholesterol levels in the plasma, severe atherosclerotic lesions, and/or LDL-C level.

Moreover, it was demonstrated in vivo that no permanent increase of AST and ALT activity levels in the plasma occurred, and thereby no liver damage was caused by the combined use of said AAV gene therapy vehicle and said molecule. Thereby, said combined use is safe for the liver.

More preferably, said at least one molecule comprises at least one of statins. Still more preferably, said at least one molecule is selected from the group consisting of Atorvastatin, Cerivastatin, Fluvastatin, Lovastatin, Mevastatin, Pitavastatin, Pravastatin, Rosuvastatin, and Simvastatin. Most preferably, said at least one molecule comprises said at least one molecule comprises Atorvastatin and/or Simvastatin.

It was found that with the further addition of said at least one statin, as described above, to said AAV gene therapy vehicle or said composition comprising said AAV gene therapy vehicle, as described above, the cholesterol level in plasma, severe atherosclerosis lesions, and/or LDL-C level is further decreased. It was also demonstrated from in vivo tests that the combined used of said AAV gene therapy vehicle or said composition comprising said AAV gene therapy vehicle, as described above, and said at least one of the statins, as described above, do not result in permanent increases of AST and ALT activity levels. Thereby, no liver damage was caused. It is therefore safe to combine said AAV gene therapy vehicle, as described above, and said at least one statin, as described above, and administer them into mammals. Said combined use is thereby safe for mammals.

According to the present invention, a kit comprising said nucleic acid for encoding said RNA molecule, as described above, is provided.

According to the present invention, a kit comprising said AAV gene therapy vehicle, as described above, is provided. Preferably, said kit comprising said AAV gene therapy vehicle, as described above, further comprises a compound reducing and/or inhibiting the cholesterol levels in plasma, LDL-C level, and/or severe atherosclerotic lesions.

According to the present invention, a kit comprising said composition comprising said AAV gene therapy vehicle, as described above, is provided.

According to the present invention, a method for manufacturing said kit, as described above, is provided.

### DRAWINGS OF THE INVENTION

FIG. 1. A schematic of Angiopoietin-like 3 (ANGPTL3) cDNA sequence with selected conserved target RNA sequences indicated (SEQ ID NOs.3-7). The sequence listed is part of NCBI Reference Sequence: NM_014495.4, nucleotides (nts) 1-1278 thereof, and represents DNA sequence (cDNA) of (part of) ANGPTL3 transcript. Hence, the corresponding RNA, has the same sequence except having instead of a T a U as depicted in FIG. 1. Nucleotides 1-542 represent exon 1, nts 543-653 represent exon 2, nts 654-768 represent exon 3, nts 769-882 represent exon 4, nts 883-978 represent exon 5 and nts 979-1245 represent exon 6. The selected target RNA sequences i.e. the DNA sequence corresponding thereto, are depicted in FIG. 1 as well. SEQ ID NO.3 corresponds with nts 139-166, in exon 1; SEQ ID NO.4 corresponds with nts 267-292, in exon 1; SEQ ID NO.5 corresponds with nts 706-728, in exon 3; SEQ ID NO.6 corresponds with nts 885-907, in exon 5 and SEQ ID NO.7 corresponds with nts 1134-1160, in exon 6.

FIG. 2. Homo sapiens pri-miR-451 from miRBase database (www.mirbase.org). (A) Twenty-two nts of the guide strand (underlined) were replaced by the mature miANG or miANG-SCR. (B) Schematic representation of the expression cassette composed of the promoter consisting the apolipoprotein E locus control region, human alpha1-antitrypsin (HCR-hAAT), the pri-miANG with 90 nts flanks on the 5′ and 3′ of the hairpin and terminated by the simian virus 40 polyadenylation (SV40 polyA) signal. (C) Schematic representation of the two Luc reporters containing ANGPTL3 target sequences downstream of the Renilla luciferase cassette (RL) and used for in vitro screening of miANG constructs.

FIG. 3. Knockdown efficacy of seventeen miANG constructs tested on Luc reporters. Human hepatocellular carcinoma cells (Huh-7) were co-transfected with 50 or 250 ng of miANG constructs and 50 ng of LucANG-A or LucANG-B reporter. Renilla (RL) and firefly (FL) luciferases were measured two days post-transfection and RL was normalized to FL expression. Scrambled (miANG-SCR1) served as negative control and was set at 100%. Data are representative of three independent experiments or two independent experiments for miANG15, miANG17 and miANG18.

FIG. 4. Knockdown potency of six miANG constructs in a titration experiment. Human hepatocellular carcinoma cells (Huh-7 were co-transfected with 10 ng LucANG-A or LucANG-B reporter and 1, 10, 50, or 250 ng of miANG constructs. Renilla (RL) and firefly (FL) luciferases were measured two days post-transfection and RL was normalized to FL expression. Scrambled (miANG-SCR1) served as negative control and was set at 100%. Data are representative of three independent experiments.

FIG. 5. ANGPTL3 mRNA knockdown in vitro upon plasmid transfection. Huh-7 were transfected with (A) 250 or (B) 400 ng of miANG5, miANG10 and miANG13 constructs. Two days post-transfection cell monolayers were collected for total RNA extraction and ANGPTL3 mRNA level was measured by TaqMan RT-QPCR. Relative ANGPTL3 mRNA levels were obtained by normalizing the data with human β-actin mRNA levels. ANGPTL3 mRNA levels in the miANG-SCR1 sample was set at 100%.

FIG. 6. Sequence distribution (%) of reads mapping to miANG5 pre-miRNA. Huh-7 were transfected with (A) 250 or (B) 400 ng of miANG5 construct. Two days post-transfection cell monolayers were collected for total RNA extraction. Results from small RNA next generation sequencing are showing the top 50 most abundant miRNAs, miANG5 expression level is highlighted.

FIG. 7. Sequence distribution (%) of reads mapping to miANG10 pre-miRNA. Huh-7 were transfected with (A) 250 or (B) 400 ng of miANG10 construct. Two days post-transfection cell monolayers were collected for total RNA extraction. Results from small RNA next generation sequencing are showing the top 50 most abundant miRNAs. miANG10 expression level is highlighted.

FIG. 8. Sequence distribution (%) of reads mapping to miANG13 pre-miRNA. Huh-7 were transfected with (A) 250 ng or (B) 400 ng of miANG13 construct. Two days post-transfection cell monolayers were collected for total RNA extraction. Results from small RNA next generation sequencing are showing the top 50 most abundant miRNAs, miANG13 expression level is highlighted.

FIG. 9. Length distribution of expressed miANG5 miRNAs determined by NGS. Huh-7 were transfected with (A) 250 ng or (B) 400 ng of miANG5 construct. Two days post-transfection cell monolayers were collected for total RNA extraction. RNA was treated with DNAse and outsourced for small RNA NGS. Reads that represented less than 2% were excluded from the figures.

FIG. 10. Length distribution of expressed miANG10 miRNAs determined by NGS. Huh-7 were transfected with (A) 250 ng or (B) 400 ng of miANG10 construct. Two days post-transfection cell monolayers were collected for total RNA extraction. RNA was treated with DNAse and outsourced for small RNA NGS. Reads that represented less than 2% were excluded from the figures.

FIG. 11. Length distribution of expressed miANG13 miRNAs determined by NGS. Huh-7 were transfected with (A) 250 ng or (B) 400 ng of miANG13 construct. Two days post-transfection cell monolayers were collected for total RNA extraction. RNA was treated with DNAse and outsourced for small RNA NGS. Reads that represented less than 2% were excluded from the figures.

FIG. 12. Vector DNA levels expressed as gc/μg of genomic DNA in livers of vehicle or AAV5-injected wild type mice.

FIG. 13. Mouse Angptl3 mRNA levels in livers of vehicle or AAV5-injected wild type mice. Data are shown as relative values to the vehicle group, which was set at 100%.

FIG. 14. miANG5 expression levels measured with (A) 24 nts assay or (B) 23 nts assay variant T in the liver of vehicle or AAV5-injected wild type mice. Data are expressed as molecules/cell. LLOQ: lower limit of quantification.

FIG. 15. ANGPTL3 protein levels (ng/ml) measured in the plasma of vehicle or AAV5-miANG5 or miANG-SCR1 injected wild type mice. Statistical analysis: ANOVA with a Dunnett's post-hoc test. *: p≤0.05, **: p≤0.01, ***: p≤0.001.

FIG. 16. Aspartate aminotransferase (AST) activity levels (mU/ml) measured in the plasma of vehicle or AAV5-miANG5 or miANG-SCR1 injected wild type mice. Statistical analysis: ANOVA with a Dunnett's post-hoc test. *: p≤0.05.

FIG. 17. Alanine aminotransferase (ALT) activity levels (mU/ml) measured in the plasma of vehicle, AAV5-miANG5 or miANG-SCR1 injected wild type mice. Statistical analysis: ANOVA with a Dunnett's post-hoc test or Kruskal-Wallis with a Dunn's post-hoc test. *: p≤0.05.

FIG. 18. Plasma levels of (A) cholesterol and (B) triglycerides in vehicle, AAV5-miANG5 or miANG-SCR1 injected wild type mice. Statistical analysis: ANOVA with a Dunnett's post-hoc. **: p≤0.01.

FIG. 19. Vector DNA levels in livers expressed as gc/μg of genomic DNA of vehicle or AAV5-injected APOE*3-Leiden.CETP mice. *

FIG. 20. Mouse Angptl3 mRNA levels in livers of vehicle or AAV5-injected APOE*3-Leiden.CETP mice. Data are shown as relative values to the vehicle group, which was set at 100%.

FIG. 21. miANG5 measured with (A) 24 nts assay or (B) 23 nts assay variant T expression levels in the liver of vehicle AAV5-injected APOE*3-Leiden.CETP mice. Data are expressed as molecules/cell. LLOQ: lower limit of quantification.

FIG. 22. Recorded (A) body weight and (B) food intake of vehicle or AAV5-injected APOE*3-Leiden.CETP mice.

FIG. 23. Measured (A) total cholesterol and (B) triglycerides in plasma of AAV5-miANG5, AAV5-miANG13 or miANG-SCR1 injected APOE*3-Leiden.CETP mice. Statistical analysis: ANOVA with a Bonferroni post-hoc test. *: p≤0.05, **: p≤0.01, ***: p≤0.001 vs vehicle group; #: p≤0.05, ##: p≤0.01, ###: p≤0.001 vs miANG-SCR1 group.

FIG. 24. Lipoprotein profiles (cholesterol and phospholipids) in plasma of AAV5-injected APOE*3-Leiden.CETP mice (A) before AAV-injection, (B) at week 4, (C) week 8, (D) week 12 and (E) week 16 post-AAV injection.

FIG. 25. Activity levels of (A) ALT and (B) AST in plasma of vehicle or AAV5-injected APOE*3-Leiden.CETP mice.

FIG. 26. Mouse ANGPTL3 protein levels in plasma of AAV5-injected APOE*3-Leiden.CETP mice. Statistical analysis: Kruskal-Wallis with a Dunn's post-hoc test. *: p≤0.05; **: p≤0.01; ***: p≤0.001.

FIG. 27. Schematic outline of the APOE*3-Leiden.CETP mice mouse study to test AAV-miANG5 in the presence or absence of atorvastatin.

FIG. 28. Schematic outline of the diet-induced dyslipidemic NHPs to test AAV5-miANG5 and simvastatin.

FIG. 29. Length distribution of expressed miANG5 miRNAs determined by NGS in APOE*3-Leiden.CETP mice injected with AAV5-miANG5 (n=5, AAV dosis: 5E+13 gc/kg). (A) mouse 11; (B) mouse 12; (C) mouse 13; (D) mouse 14 and (E) mouse 15. Total RNA was isolated from liver samples, treated with DNAse and outsourced for small RNA NGS. Reads that represented less than 2% were excluded from the figures.

FIG. 30. Vector DNA levels expressed as gc/μg of genomic DNA in livers of vehicle or AAV5-injected (alone or in combination with atorvastatin) APOE*3-Leiden.CETP mice. LLOQ: lower limit of quantification.

FIG. 31. Mouse Angptl3 mRNA levels in livers of vehicle or AAV5-injected (alone or in combination with atorvastatin) APOE*3-Leiden.CETP mice. Data are shown as relative values to the vehicle group, which was set at 100%.

FIG. 32. miANG5 (23 nts) expression levels in the liver of vehicle AAV5-injected (alone or in combination with atorvastatin) APOE*3-Leiden.CETP mice. Data are expressed as molecules/cell. LLOQ: lower limit of quantification.

FIG. 33. Recorded (A) body weight and (B) food intake of vehicle or AAV5-injected (alone or in combination with atorvastatin) APOE*3-Leiden.CETP mice.

FIG. 34. Measured (A) plasma total cholesterol levels and calculated (B) cholesterol exposure of vehicle, AAV5-miANG5 or miANG-SCR1 (alone or in combination with atorvastatin) injected APOE*3-Leiden.CETP mice. Statistical analysis: ANOVA with a Bonferroni post-hoc test. *: p≤0.05, **: p≤0.01, ***: p≤0.001 vs vehicle group; #: p≤0.05, ##: p≤0.01, ###: p≤0.001 vs miANG-SCR1 group; (A) &&: p≤0.01, &&&: p≤0.001 vs miANG-SCR1+atorvastatin group; (B) &&&: p≤0.001.

FIG. 35. Measured (A) plasma triglyceride levels and calculated (B) triglyceride exposure of vehicle, AAV5-miANG5 or miANG-SCR1 (alone or in combination with atorvastatin) injected APOE*3-Leiden.CETP mice Statistical analysis: ANOVA with a Bonferroni post-hoc test. **: p≤0.01, ***: p≤0.001 vs vehicle group; ##: p≤0.01, ###: p≤0.001 vs miANG-SCR1 group; (A) &&: p≤0.01, &&&: p≤0.001 vs miANG-SCR1+atorvastatin group; (B) &&&: p≤0.001.

FIG. 36. Pooled measurements of cholesterol lipoprotein profiles in plasma of AAV5-injected APOE*3-Leiden.CETP mice (A) before AAV-injection, (B) at week 4, (C) week 8 and (D) week 12 post-AAV injection.

FIG. 37. Pooled measurements of phospholipid lipoprotein profiles in plasma of AAV5-injected APOE*3-Leiden.CETP mice (A) before AAV-injection, (B) at week 4, (C) week 8 and (D) week 12 post-AAV injection.

FIG. 38. Individual measurements of (A) cholesterol, (B) phospholipid and (C) triglyceride lipoprotein profiles in plasma of AAV5-injected APOE*3-Leiden.CETP mice at week 16 post-AAV injection.

FIG. 39. ANGPTL3 protein levels (ng/ml) measured in the plasma of vehicle or AAV5-injected (alone or in combination with atorvastatin) APOE*3-Leiden.CETP mice. Statistical analysis: ANOVA with a Bonferroni post-hoc test or Kruskal-Wallis with a Dunn's post-hoc test. *: p≤0.05; **: p≤0.01; ***: p≤0.001.

FIG. 40. Activity levels of (A) ALT and (B) AST in plasma of vehicle or AAV5-injected (alone or in combination with atorvastatin) APOE*3-Leiden.CETP mice.

FIG. 41. Measured atherosclerosis total lesion area in the aortic root of vehicle or AAV5-injected (alone or in combination with atorvastatin) APOE*3-Leiden.CETP mice. Statistical analysis: Kruskal-Wallis test followed by individual Mann-Whitney tests. **: p≤0.01, ***: p≤0.001 vs vehicle group; ###: p≤0.001 vs miANG-SCR1 group; AAA: p≤0.001; &&&: p≤0.001.

FIG. 42. Determined atherosclerotic (A) lesion severity (B) number of lesions per cross section of vehicle or AAV5-injected (alone or in combination with atorvastatin) APOE*3-Leiden.CETP mice. Statistical analysis: Kruskal-Wallis test followed by individual Mann-Whitney tests. *: p≤0.05, **: p≤0.01, ***: p≤0.001 vs vehicle group; #: p≤0.05, ##: p≤0.01, ###: p≤0.001 vs miANG-SCR1 group; &: p≤0.05, &&: p≤0.01; \hat{ }: p≤0.05, \hat{ }\hat{ }: p≤0.01.

FIG. 43. Plasma lipid levels (triglycerides, LDL-Cholesterol, HDL-Cholesterol and Total-Cholesterol) of AAV5-miANG5 and vehicle treated dyslipidemic NHPs. C1-C3: vehicle treated animals. T1-T5: AAV5-miANG5-treated animals. Between day −40 till −27 prior to dosing and from day 57 till 84 post-dosing, animals were co-administered with Simvastatin.

FIG. 44. The mean percentage change 90 days post-treatment compared to the pre-treatment levels (baseline) was calculated for the control and AAV5-miANG5 treated groups. To calculate this, the area under the curve was calculated for a period of 90 pre- and post-treatment.

FIG. 45. Plasma ALT and AST levels in vehicle or AAV5-miANG5 treated NHPs. C1-C3: vehicle treated animals. T1-T5: AAV5-miANG5-treated animals.

### DETAILED DESCRIPTION OF THE INVENTION

The present invention relates to gene therapy, and in particular to the use of RNA interference (RNAi) in gene therapy for targeting RNA encoded by the Angiopoietin-like 3 (ANGPTL3) gene, preferably by the human ANGPTL3 gene (OMIM: 604774, https://www.omim.org/).

One objective of the present invention provides an RNA molecule comprising a first RNA sequence and a second RNA sequence, wherein said first RNA sequence comprises a sequence that is substantially complementary to a target RNA sequence comprised in an RNA encoded by an ANGPTL3 gene, wherein said sequence complementary to said target RNA sequence has at least 19 nucleotides.

The term “substantially complementary”, as used herein, refers to that two nucleic acid sequences are complementary and antiparallel to each other, and thereby the two nucleic acid sequences bind to each other. The term “substantially” means that the complementarity between the two sequences is sufficient to bind to each other for an amount of time sufficient to have an at least partial inhibitory effect. It is preferred of course that the complementarity is complete, but some gaps and/or mismatches may be allowed. The number of mismatches should be no higher than 10%. The important feature is that the complementarity is sufficient to allow for binding of the two strands in situ. The binding must be strong enough to exert an inhibitory effect.

The complete or partial first RNA sequence, as described above, is in a guide strand, which is also referred to as antisense strand as it is complementary (“anti”) to a sense target RNA sequence. The sense target RNA sequence is comprised in an RNA encoded by an ANGPTL3 gene.

Said second RNA sequence, as described herein, refers to as “sense strand”, having substantially identical sequence identity to said target RNA sequence, as described herein. The first and second RNA sequences are comprised in a double stranded RNA and are substantially complementary. Said double stranded RNA according to the invention is to induce RNAi, thereby reducing expression of ANGPTL3 transcripts.

In said RNA molecule, as described above, the sequence comprised in the first RNA sequence optionally has at most 4 nucleotides, 5 nucleotides, or 6 nucleotides different from a complementary sequence of said target sequence comprised in an RNA encoded by the ANGPTL3 gene, preferably the human ANGPTL3 gene. Optionally, the sequence comprised in the first RNA sequence optionally has at least 1 nucleotide, 2 nucleotides, or 3 nucleotides different from a complementary sequence of said target sequence comprised in an RNA encoded by the ANGPTL3 gene, preferably the human ANGPTL3 gene. Optionally, said sequence comprised in the first RNA sequence is identical to a complementary sequence of said target sequence comprised in an RNA encoded by the ANGPTL3 gene, preferably the human ANGPTL3 gene.

Thereby, said RNA molecule, as described above, is capable of inducing RNAi, and is thereby sequence-specifically binding to a sequence comprising the target RNA sequence. Hence, said sequence comprised in said first RNA sequence, as described above, has a sequence-specific binding to said target RNA sequence encoded by the ANGPTL3 gene

Preferably, said ANGPTL3 gene, as described herein, is a mammalian ANGPTL3 gene. More preferably, said ANGPTL3 gene is a mouse ANGPTL3 gene, or a non-human primate (NHP) ANGPTL3 gene. Most preferably, said ANGPTL3 gene is a human ANGPTL3 gene (OMIM:604774).

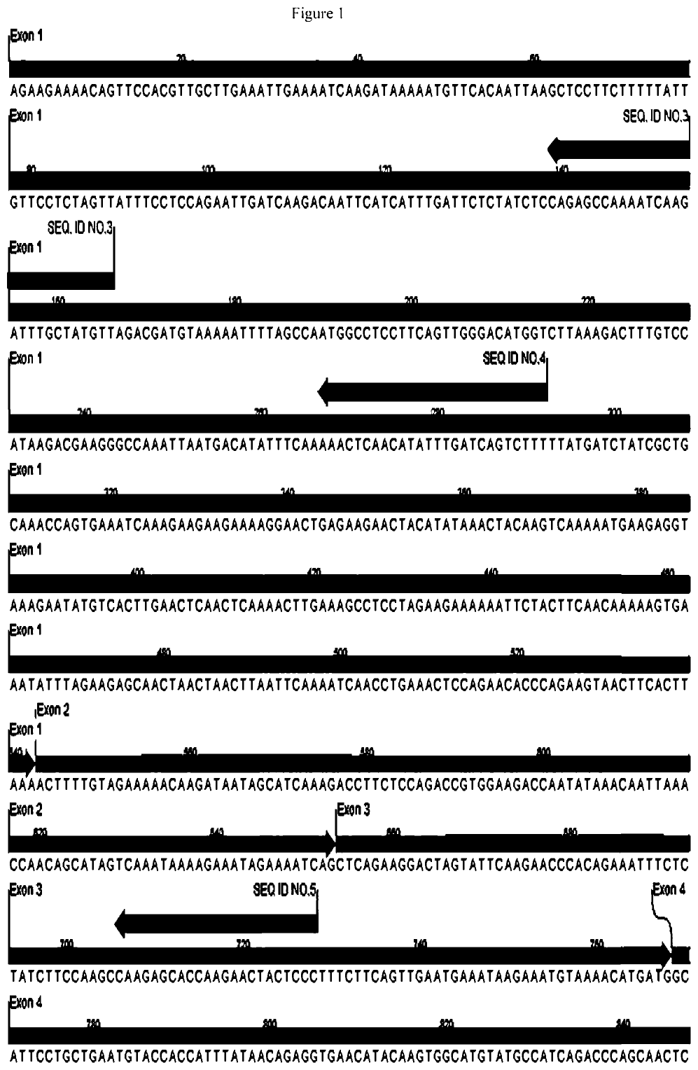

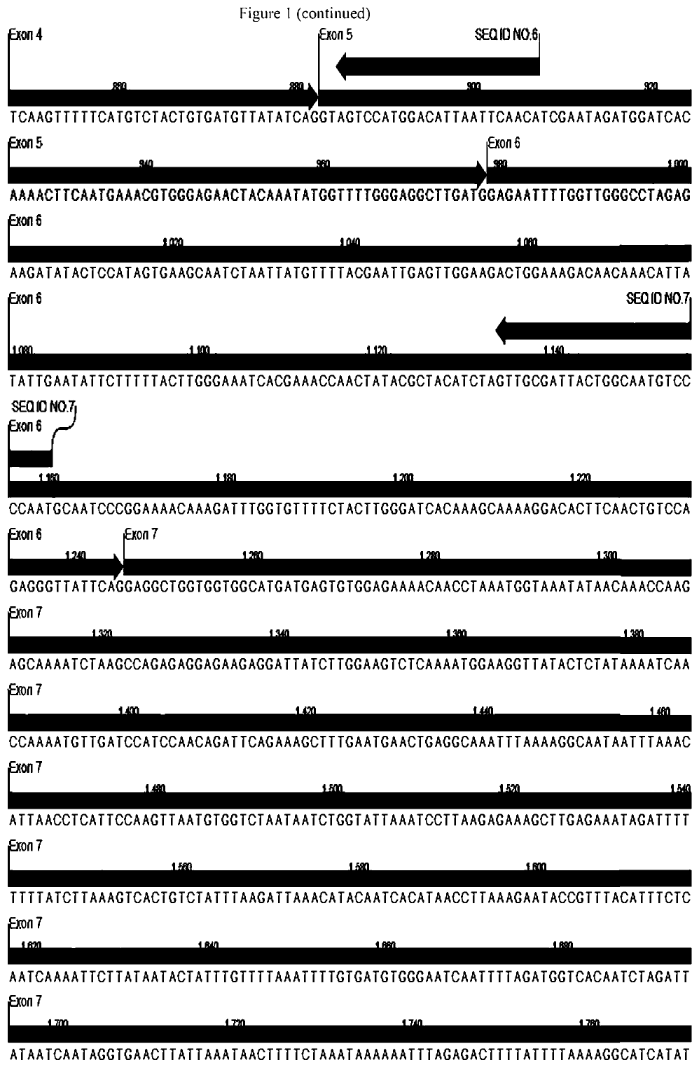

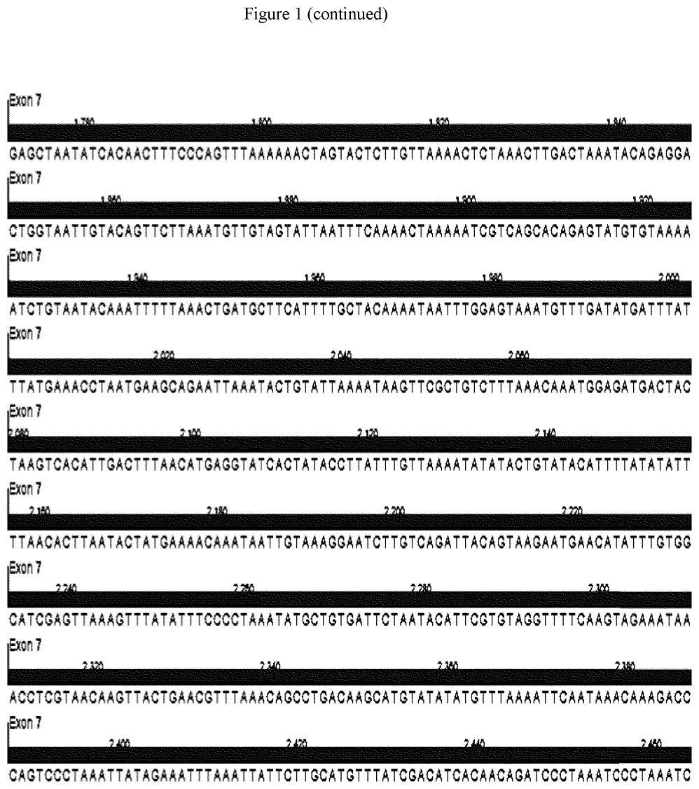

Preferably, said target RNA sequence, as described herein, is comprised in a RNA sequence encoded by the DNA sequence as shown in Figure. 1 (nucleotides 1-2926 of SEQ ID. NO.2). Said DNA sequence encodes a spliced mRNA of the human ANGPTL3 gene.

According to the present invention, SEQ ID. NO.1 is used as a reference gene sequence for the ANGPTL3 gene (i.e. NCBI Reference Sequence: NG_028169.1). Thereby, exon 1-7 sequences of SEQ ID. NO. 1 correspond to exon 1-7 of SEQ ID. NO. 2, as shown in FIG. 1. That is, Exon 1 of SEQ ID. NO. 1 corresponds to nucleotides 5005-5546 of SEQ ID. NO. 2; Exon 2 of SEQ ID. NO. 1 corresponds to nucleotides 6181-6291 of SEQ ID. NO. 2; Exon 3 of SEQ ID. NO. 1 corresponds to nucleotides 8567-8681 of SEQ ID. NO. 2; Exon 4 of SEQ ID. NO. 1 corresponds to nucleotides 9254-9367 of SEQ ID. NO. 2; Exon 5 of SEQ ID. NO. 1 corresponds to nucleotides 9770-9865 of SEQ ID. NO. 2; Exon 6 of SEQ ID. NO. 1 corresponds to nucleotides 11454-11720 of SEQ ID. NO. 2, and Exon 7 of SEQ ID. NO. 1 corresponds to nucleotides 12118-13798 of SEQ ID. NO. 2.

The term “at least one”, as described herein, refers to that the indicated subject, such as the exon, as described herein, is in the amount of one, two, three, or more.

The term “conserved sequence” or “conserved region”, as described herein, refers to a short length of sequence which can be found in various species with a high level of similarity. A conserved sequence can be identified through aligning a number of nucleic acid sequences from various species for encoding the same RNA or the same protein, and thereby a part of the sequences can be found to be substantially identical. The term “conserved sequence” is also known as “conservative sequence” or “conserved region”.

The term “exon”, as described herein, refers to a region of the genes that encode proteins.

Said target sequence encoded by the ANGPTL3 gene, as described above, is designed to comprise a complete or a part of at least one conserved sequence encoded by the ANGPTL3 gene. A number of the conserved sequences of the ANGPTL3 gene are identified and selected for the present invention.

Preferably, the conserved sequences are comprised in exon 1, exon 3, exon 5 or exon 6 of the ANGPTL3 gene, as described above. More preferably, said target sequence, as described above, comprises a complete or a part of the conserved sequence in exon 1, exon 5 or exon 6 of the ANGPTL3 gene, as described above. Yet more preferably, said target sequence, as described above, comprises a complete or a part of the conserved sequence in exon 1 or exon 5 of the ANGPTL3 gene, as described above.

As described above, the RNA molecule according to the present invention knocks down the transcripts of the ANGPTL3 gene. Moreover, the RNA molecule, as described above, improves the “off-target” issue typically present in RNAi-based gene therapies. The “off-target” issue, as described herein, refers to that said second RNA sequence of the RNA molecule, as described herein, binds to an unintended target RNA sequence. Thereby, the RNA molecule, as described above, can suppress or inhibit the transcripts of the ANGPTL3 gene effectively.

Moreover, with the RNA molecule, as described above, medical practitioners and/or patients can administer said RNA molecule, a composition, an AAV vehicle or a formulation comprising said RNA molecule into the human body in a convenient and simple manner. Thereby, the RNA molecule, as described above, meets the aforementioned needs for the treatment and/or prevention of Dyslipidemia. Preferably, in said RNA molecule, as described above, said first RNA sequence is substantially complementary to said second RNA sequence, as described above.

Said RNA molecule, as described above, preferably includes an RNA hairpin or a double-stranded RNA (dsRNA). More preferably, said RNA molecule includes miR-451.

The term “RNA hairpin”, as described herein, refers to a secondary structure of an RNA, which comprises two strands which are complementary to each other and also comprises a loop which connects the two strands. An RNA hairpin can guide RNA folding, determine interactions in a ribozyme, protect messenger RNA (mRNA) from degradation, serve as a recognition motif for RNA binding protein.

The term “dsRNA”, as described herein, refers to two nucleic acid strands which are complementary and antiparallel to each other. The two strands are stabilized by hydrogen bonds.

The term “shRNA”, as described herein, refers to an artificial RNA molecule with a hairpin structure which can be used in RNAi for degrading or cleaving a target mRNA or suppress the translation of the target mRNA.

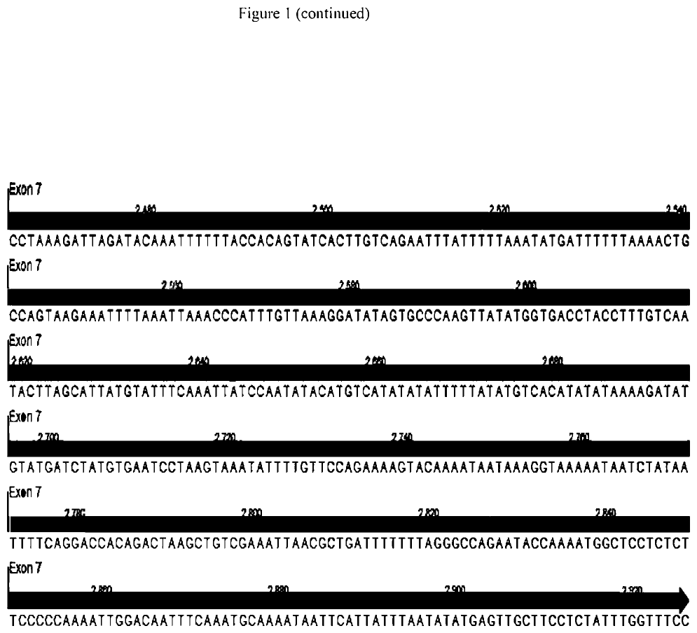

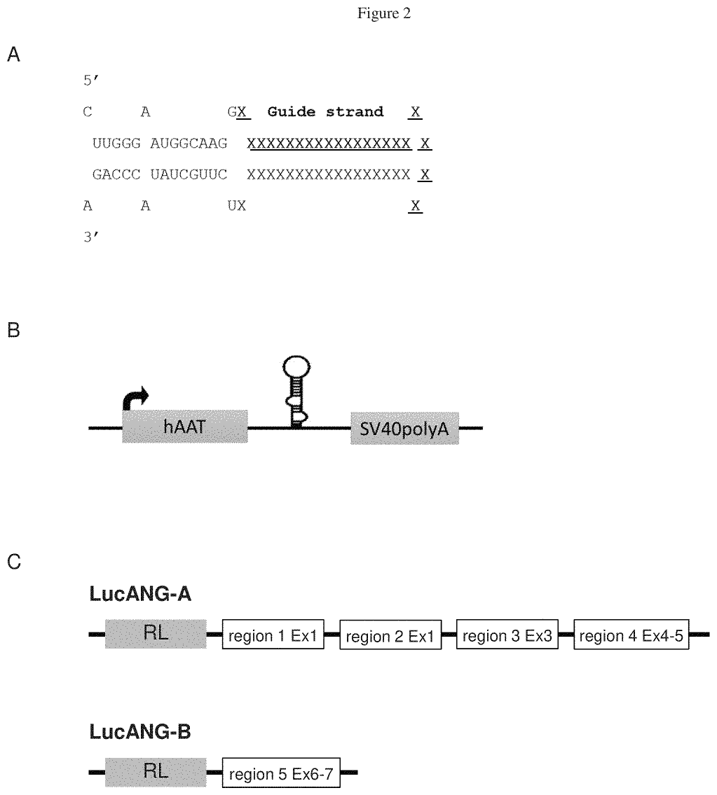

The term “miR-451”, as described herein, refers to a specific scaffold obtained from microRNA 451a. The pri-miRNA scaffold for miR-451 is depicted in FIG. 2.A. This scaffold allows to induce RNAi, in particularly that RNAi is induced by the guide strand of this scaffold. The pri-miR451 scaffold does not result in a passenger strand because the processing is different from the canonical miRNA processing pathway (Cheloufi, S. et. al., 2010 and Yang, J. S. et. al., 2010). Thereby, the use of miR-451 can prevent or reduce the possibility of having unwanted potential off-targeting by passenger strands.

In said RNA molecule, as described above, said sequence comprised in the first RNA sequence has optionally at least 15 nucleotides, optionally at least 16 nucleotides, optionally at least 17 nucleotides, optionally at least 18 nucleotides, optionally at least 19 nucleotides, optionally at least 22 nucleotides, or optionally at least 24 nucleotides. Also, said sequence comprised in the first RNA sequence, as described above, has optionally at most 30 nucleotides, optionally at most 28 nucleotides, or optionally at most 26 nucleotides.

Preferably, said sequence comprised in the first RNA sequence, as described above, is one selected from the group consisting of SEQ ID NOs. 8-25. More preferably, said sequence comprised in the first RNA sequence, as described above, is one selected from the group consisting of SEQ ID NOs. 8-17 and 19-25. Still more preferably, said sequence comprised in the first RNA sequence is one selected from the group consisting of SEQ ID NOs. 11, 12, 16, 17, 20 and 25. Yet more preferably, said sequence comprised in the first RNA sequence is one selected from the group consisting of SEQ ID NOs. 11, 12, 17, 20 and 25. Most preferably, said sequence comprised in the first RNA sequence includes SEQ ID NO. 12. Still most preferably, said sequence comprised in the first RNA sequence, as described above, consists SEQ ID NO. 12.

TABLE 1
sequence comprised in said First RNA sequences

| SEQ | FIRST RNA SEQUENCE | length |
|---|---|---|
| ID NO. | (5′-sequence-3′) | (nucleotides) |
| 8 | AACAUAGCAAAUCUUGAUUUUG | 22 |
| 9 | ACAUAGCAAAUCUUGAUUUUGG | 22 |
| 10 | CAUAGCAAAUCUUGAUUUUGGC | 22 |
| 11 | AUAGCAAAUCUUGAUUUUGGCU | 22 |
| 12 | UAGCAAAUCUUGAUUUUGGCUC | 22 |
| 13 | AGCAAAUCUUGAUUUUGGCUCU | 22 |
| 14 | GCAAAUCUUGAUUUUGGCUCUG | 22 |
| 15 | GACUGAUCAAAUAUGUUGAGUU | 22 |
| 16 | AGACUGAUCAAAUAUGUUGAGU | 22 |
| 17 | AAGACUGAUCAAAUAUGUUGAG | 22 |
| 18 | GGGAGUAGUUCUUGGUGCUCUU | 22 |
| 19 | GGAGUAGUUCUUGGUGCUCUUG | 22 |
| 20 | UGUUGAAUUAAUGUCCAUGGAC | 22 |
| 21 | GUUGAAUUAAUGUCCAUGGACU | 22 |
| 22 | GGGGACAUUGCCAGUAAUCGCA | 22 |
| 23 | GGGACAUUGCCAGUAAUCGCAA | 22 |
| 24 | GGACAUUGCCAGUAAUCGCAAC | 22 |
| 25 | AUUGGGGACAUUGCCAGUAAUC | 22 |

In said RNA molecule, as described above, said first RNA sequence comprises a sequence which is substantially complementary to said target sequence, as described herein.

Said sequence comprised in the first RNA sequence, as described above, is designed based on one of the conserved sequences comprised in one of the exons, as described above.

Such a first RNA sequence is combined with a second RNA sequence. A skilled person is well capable of designing and selecting a suitable second RNA sequence to combine with said first RNA sequence, as described above, that induces RNAi in a cell. Suitable second RNA sequences are listed below in Table 2.

TABLE 2
second RNA sequences

| SEQ | SECOND RNA SEQUENCE | length |
|---|---|---|
| ID NO. | (5′-sequence-3′) | (nucleotides) |
| 26 | AUCAAGAUUUGCUAUGU | 17 |
| 27 | AAUCAAGAUUUGCUAUG | 17 |
| 28 | AAAUCAAGAUUUGCUAU | 17 |
| 29 | AAAAUCAAGAUUUGCUA | 17 |
| 30 | CAAAAUCAAGAUUUGCU | 17 |
| 31 | CCAAAAUCAAGAUUUGC | 17 |
| 32 | GCCAAAAUCAAGAUUUG | 17 |
| 33 | CAACAUAUUUGAUCAGU | 17 |
| 34 | AACAUAUUUGAUCAGUC | 17 |
| 35 | ACAUAUUUGAUCAGUCU | 17 |
| 36 | GCACCAAGAACUACUCC | 17 |
| 37 | AGCACCAAGAACUACUC | 17 |
| 38 | AUGGACAUUAAUUCAAC | 17 |
| 39 | CAUGGACAUUAAUUCAA | 17 |
| 40 | AUUACUGGCAAUGUCCC | 17 |
| 41 | GAUUACUGGCAAUGUCC | 17 |
| 42 | CGAUUACUGGCAAUGUC | 17 |
| 43 | ACUGGCAAUGUCCCCAA | 17 |

Preferably, said first RNA sequence is comprised in a miRNA scaffold, more preferably a miR-451 scaffold.

A preferred scaffold comprising said first and second RNA sequences, as described above, comprises a sequence which is one selected from the group of sequences listed in Tables. 3 and 4.

Optionally, the sequences as listed in Table. 3 comprise further sequences. Also optionally, the sequences as listed in Table. 3 are comprised in the sequence of a pri-miRNA scaffold, preferably the pri-miRNA scaffold in Table. 4.

TABLE 3
Combination of first and second RNA sequences.

| SEQ | First RNA sequence- | length |
|---|---|---|
| ID | second RNA sequence | (nucleo- |
| NO. | [5′-sequence-3′] | tides) |
| 44 | AACAUAGCAAAUCUUGAUUUUGAUCAAGAUUUGCUAUGU | 39 |
| 45 | ACAUAGCAAAUCUUGAUUUUGGAAUCAAGAUUUGCUAUG | 39 |
| 46 | CAUAGCAAAUCUUGAUUUUGGCAAAUCAAGAUUUGCUAU | 39 |
| 47 | AUAGCAAAUCUUGAUUUUGGCUAAAAUCAAGAUUUGCUA | 39 |
| 48 | UAGCAAAUCUUGAUUUUGGCUCCAAAAUCAAGAUUUGCU | 39 |
| 49 | AGCAAAUCUUGAUUUUGGCUCUCCAAAAUCAAGAUUUGC | 39 |
| 50 | GCAAAUCUUGAUUUUGGCUCUGGCCAAAAUCAAGAUUUG | 39 |
| 51 | GACUGAUCAAAUAUGUUGAGUUCAACAUAUUUGAUCAGU | 39 |
| 52 | AGACUGAUCAAAUAUGUUGAGUAACAUAUUUGAUCAGUC | 39 |
| 53 | AAGACUGAUCAAAUAUGUUGAGACAUAUUUGAUCAGUCU | 39 |
| 54 | GGGAGUAGUUCUUGGUGCUCUUGCACCAAGAACUACUCC | 39 |
| 55 | GGAGUAGUUCUUGGUGCUCUUGAGCACCAAGAACUACUC | 39 |
| 56 | UGUUGAAUUAAUGUCCAUGGACAUGGACAUUAAUUCAAC | 39 |
| 57 | GUUGAAUUAAUGUCCAUGGACUCAUGGACAUUAAUUCAA | 39 |
| 58 | GGGGACAUUGCCAGUAAUCGCAAUUACUGGCAAUGUCCC | 39 |
| 59 | GGGACAUUGCCAGUAAUCGCAAGAUUACUGGCAAUGUCC | 39 |
| 60 | GGACAUUGCCAGUAAUCGCAACCGAUUACUGGCAAUGUC | 39 |
| 51 | AUUGGGGACAUUGCCAGUAAUCACUGGCAAUGUCCCCAA | 39 |

TABLE 4
pri-miRNA sequences

| SEQ ID | flank-first RNA sequence- | length |
|---|---|---|
| NO. | second RNA sequence-flank [5′-NNNN-3′] | |
| 62 | CUUGGGAAUGGCAAGGAACAUAGCAAAUCUUGAUUUUG | 72 |
| | AUCAAGAUUUGCUAUGUCUCUUGCUAUACCCAGA | |
| 63 | CUUGGGAAUGGCAAGGACAUAGCAAAUCUUGAUUUUGG | 72 |
| | AAUCAAGAUUUGCUAUGCUCUUGCUAUACCCAGA | |
| 64 | CUUGGGAAUGGCAAGGCAUAGCAAAUCUUGAUUUUGGC | 72 |
| | AAAUCAAGAUUUGCUAUCUCUUGCUAUACCCAGA | |
| 65 | CUUGGGAAUGGCAAGGAUAGCAAAUCUUGAUUUUGGCU | 72 |
| | AAAAUCAAGAUUUGCUACUCUUGCUAUACCCAGA | |
| 66 | CUUGGGAAUGGCAAGGUAGCAAAUCUUGAUUUUGGCUC | 72 |
| | CAAAAUCAAGAUUUGCUCUCUUGCUAUACCCAGA | |
| 67 | CUUGGGAAUGGCAAGGAGCAAAUCUUGAUUUUGGCUCU | 72 |
| | CCAAAAUCAAGAUUUGCCUCUUGCUAUACCCAGA | |
| 68 | CUUGGGAAUGGCAAGGGCAAAUCUUGAUUUUGGCUCUG | 72 |
| | GCCAAAAUCAAGAUUUGAUCUUGCUAUACCCAGA | |
| 69 | CUUGGGAAUGGCAAGGGACUGAUCAAAUAUGUUGAGUU | 72 |
| | CAACAUAUUUGAUCAGUAUCUUGCUAUACCCAGA | |
| 70 | CUUGGGAAUGGCAAGGAGACUGAUCAAAUAUGUUGAGU | 72 |
| | AACAUAUUUGAUCAGUCCUCUUGCUAUACCCAGA | |
| 71 | CUUGGGAAUGGCAAGGAAGACUGAUCAAAUAUGUUGAG | 72 |
| | ACAUAUUUGAUCAGUCUCUCUUGCUAUACCCAGA | |
| 72 | CUUGGGAAUGGCAAGGGGGAGUAGUUCUUGGUGCUCUU | 72 |
| | GCACCAAGAACUACUCCAUCUUGCUAUACCCAGA | |
| 73 | CUUGGGAAUGGCAAGGGGAGUAGUUCUUGGUGCUCUUG | 72 |
| | AGCACCAAGAACUACUCAUCUUGCUAUACCCAGA | |
| 74 | CUUGGGAAUGGCAAGGUGUUGAAUUAAUGUCCAUGGAC | 72 |
| | AUGGACAUUAAUUCAACCUCUUGCUAUACCCAGA | |
| 75 | CUUGGGAAUGGCAAGGGUUGAAUUAAUGUCCAUGGACU | 72 |
| | CAUGGACAUUAAUUCAAAUCUUGCUAUACCCAGA | |
| 76 | CUUGGGAAUGGCAAGGGGGGACAUUGCCAGUAAUCGCA | 72 |
| | AUUACUGGCAAUGUCCCAUCUUGCUAUACCCAGA | |
| 77 | CUUGGGAAUGGCAAGGGGGACAUUGCCAGUAAUCGCAA | 72 |
| | GAUUACUGGCAAUGUCCAUCUUGCUAUACCCAGA | |
| 78 | CUUGGGAAUGGCAAGGGGACAUUGCCAGUAAUCGCAAC | 72 |
| | CGAUUACUGGCAAUGUCAUCUUGCUAUACCCAGA | |
| 79 | CUUGGGAAUGGCAAGGAUUGGGGACAUUGCCAGUAAUC | 72 |
| | ACUGGCAAUGUCCCCAACUCUUGCUAUACCCAGA | |

In said RNA molecule, as described above, said target sequence is comprised in an RNA encoded by said ANGPTL3 gene. Preferably, said target sequence comprised in an RNA encoded by a part of at least one exon is comprised in said ANGPTL3 gene. Still preferably, said exon, as described above, is exon 1, exon 3, exon 5, or exon 6, comprised in the ANGPTL3 gene. More preferably, said exon, as described above, is exon 1, exon 5, or exon 6, comprised in the ANGPTL3 gene. Still more preferably, said target sequence comprised in an RNA is encoded by said ANGPTL3 gene. Preferably, said target sequence comprised in an RNA is encoded by at least one conserved sequence comprised in one exon, as described above, comprised in said ANGPTL3 gene. Preferably, the conserved sequence (NCBI reference sequence: NM_014495.4: position 139-166 nucleotides, hereafter referred to as SEQ ID NO.3) is comprised in exon 1 of the ANGPTL3 gene, the conserved sequence (NCBI reference sequence: NM_014495.4: position 267-292 nucleotides, hereafter referred to as SEQ ID NO.4) is comprised in exon 1 of the ANGPTL3 gene, the conserved sequence (NCBI reference sequence: NM_014495.4: position 706-728 nucleotides, hereafter referred to as SEQ ID NO.5) is comprised in exon 3 of the ANGPTL3 gene, the conserved sequence (NCBI reference sequence: NM_014495.4: position 885-907 nucleotides, hereafter referred to as SEQ ID NO 6) is comprised in exon 5, or the conserved sequence (NCBI reference sequence: NM_014495.4: position 1134-1160 nucleotides, hereafter referred to as SEQ ID NO.7) is comprised in exon 6.

TABLE 5
Suitable target RNA sequence
of the present invention

| SEQ | | length |
|---|---|---|
| ID. | TARGET RNA SEQUENCE | (nucleo- |
| NO. | (5′-sequence-3′) | tides) |
| 3 | CAGAGCCAAAAUCAAGAUUUGCUAUGUU | 28 |
| 4 | AAACUCAACAUAUUUGAUCAGUCUUU | 26 |
| 5 | CAAGAGCACCAAGAACUACUCCC | 23 |
| 6 | AGUCCAUGGACAUUAAUUCAACA | 23 |
| 7 | GUUGCGAUUACUGGCAAUGUCCCCAAU | 27 |

SEQ ID. NO.s. 3-7 comprised in exons in ANGPTL3 gene (NCBI Reference Sequence: NM_014495.4 (SEQ ID NO.2)). SEQ ID NO.3: 139-166 in exon 1; SEQ ID NO.4: 267-292, exon 1; SEQ ID NO.5: 706-728 in exon 3; SEQ ID. NO.6: 885-907 in exon 5; SEQ ID. NO.7: 1134-1160, exon 6. Target RNA sequences SEQ ID. NO.s. 3, 4, 5 and 6 are fully or essentially conserved in a number of animals, such as human, monkey, mouse and rat. Target RNA sequence SEQ ID NO.7 was selected as indicated by (Graham, M. J. et al., 2017) to be the target RNA sequence for antisense oligonucleotide (ASO) IONIS-ANGPTL3-L_{RX}.

One of the objectives of the present invention is to provide a composition comprising said RNA, as described above or a nucleic acid encoding said RNA, as described above, or an AAV gene therapy vehicle comprising said RNA.

The term “plasma cholesterol levels” or “cholesterol levels in the plasms” as used above and herein, refers to the amount of cholesterol present in the plasm.

The term “TC” as used above and herein, refers to the amount of cholesterol present in the plasma and serum.

Optionally, said composition further comprises an additive, wherein said additive is for further enhancing the stability of said composition, such as for longer shelf-life, easy storage, easy transportation, and/or less degradations.

The term “additive” as described above and herein, refers to a substance further added into said composition, as described above, in order to further enhance the properties of said composition or to act as a filler without altering or affecting the effectiveness and/or the properties of said composition, as described above.

One of the objectives of the present invention is the use of said composition, as described above as a medicament. Preferably, said composition, as described above, is used as a medicament for knocking down the transcripts of the ANGPTL3 gene, as described above.

The term “transcripts” as used above and herein, refers to gene products encoded by a gene, such as the ANGPTL3 gene, as described above. Said gene products includes the RNA encoded from the ANGPTL3 gene, as described above, and the proteins encoded from the ANGPTL3 gene.

The term “knockdown”, “knock down” or “knocking down”, as used herein, refers to that the level of the transcripts of the ANGPTL3 gene, as described above, is lowered, reduced, suppressed, and/or decreased. Also, the term “knockdown”, “knock down” or “knocking down”, as used herein, refers to that the level of the transcripts of the ANGPTL3 gene, as described above, is inhibited or silenced.

The RNA molecule, as described above, knocks down the transcripts of the ANGPTL3 gene. Hence, the composition, as described above, reduces and/or inhibits the levels of phospholipids, plasma cholesterol levels, LDL-C, TC, and/or TG and/or reduce and/or inhibit initial, mild, and/or severe atherosclerotic lesions in the human body, and thereby said composition, as described above, is used for the treatment and/or prevention of lipid and/or lipoprotein metabolic disorder. Preferably, the lipid and/or lipoprotein metabolic disorders including hyperlipidemias such as familial hypercholesterolemia, LDL-hypercholesterolemia, hypertriglyceridemia, mixed hyperlipoproteinemia, and nonalcoholic steatohepatitis (NASH). Preferably, the composition, as described above, is used as a medicament for the treatment and/or prevention of Dyslipidemia.

The term “atherosclerotic lesions” means the lesion severity and/or the lesion size of atherosclerosis. Said atherosclerotic lesions are classified into five categories according to the American Heart Association (Stary, H. C. et al., 1995; Stary, H. C., 2000): type I is early fatty streak; type II is regular fatty streak; type III is mild plaque; type IV is moderate plaque; type V is severe plaque. The atherosclerotic lesions, as used above and herein, comprise initial lesions, mild lesions, and/or severe lesions.

The term “initial lesions”, as described above and herein, is referred to as comprising early and regular fatty streaks. That also means that the initial lesions comprise types I-II according to the American Heart Association (Stary, H. C. et al., 1995; Stary, H. C., 2000).

The term “mild lesions”, as describe above and herein, is referred to as comprising mild plaque. That also means that the mild lesions comprise type III according to the American Heart Association (Stary, H. C. et al., 1995; Stary, H. C., 2000).

The term “severe lesions”, as described herein, is referred to as comprising moderate plaque and severe plaque. That also means that the severe lesions comprise types IV and V according to the American Heart Association (Stary, H. C. et al., 1995; Stary, H. C., 2000).

One objective of the present invention is to provide a DNA expression cassette.

The term “DNA expression cassette”, as described herein, refers to a DNA nucleic acid sequence comprising a gene or a nucleic acid sequence encoding an RNA molecule, a promoter, and a nucleic acid sequence encoding a poly A tail. Said DNA expression cassette is flanked by ITRs and is comprised in a virus vehicle and subsequently delivered to a target organ, such as the liver.

The term “RNA molecule”, as used herein, refers to a hairpin, a double stranded RNA (dsRNA), small interfering RNA (siRNA), and microRNA (miRNA). Said hairpin is preferably a short hairpin RNA (shRNA) or long hairpin RNA (lhRNA). More preferably, said RNA molecule is miR-451 or an RNA molecule encoded by SEQ ID NO 124.

The term “promoter”, as used herein, refers to a DNA sequence that is typically located at the 5′ end of transcription initiation site for driving or initiating the transcription of a linked nucleic acid sequence. Preferably, said promoter includes a liver-specific promoter as ANGPTL3 is expressed mainly in the liver.

More preferably, said promoter, as described above, is selected from the group consisting of pol I promoter, pol II promoter, pol III promoter, an inducible or repressible promoter, an al-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, HLP, minimal TTR promoter, FVIII promoter, hyperon enhancer, ealb-hAAT, EF1-Alpha promoter, Herpes Simplex Virus Tymidine Kinase (TK) promoter, U1-1 snRNA promoter, Apolipoprotein promoter, TRE promoter, rtTA-TRE (inducible promoter), LP1 promoter, Q1 promoter, Q1-prime promoter, C14 promoter, C16 promoter or any synthetic promoter selected from SEQ ID NOs 84-87 and 108-109 and 112-115 and variants thereof.

Optionally, each of said variants of SEQ ID NOs 84-87 and 108-109 and 112-115, as described above, have sequences essentially identical to SEQ ID NOs 84-87 and 108-109 and 112-115, respectively, and said variants have substantially the same function as SEQ ID NOs 84-87 and 108-109 and 112-115.

Optionally, each of said variants of SEQ ID NOs 84-87 and 108-109 and 112-115, as described above, has a nucleic acid sequence comprising at least 1, 2, 3, 4, or 5 nucleotides different from the sequences of SEQ ID NOs 84-87 and 108-109 and 112-115.

Optionally, each of said variants of SEQ ID NOs 84-87 and 108-109 and 112-115, as described above, has a nucleic acid sequence comprising at most 40, 35, 30, 25, or 20 nucleotides different from the sequence of SEQ ID NOs 84-87 and 108-109 and 112-115.

The term “poly A tail”, as described herein, refers to a long chain of adenine nucleotides that is added to a mRNA molecule for increasing the stability of the RNA molecule. Preferably, the poly A tail is the simian virus 40 polyadenylation (SV40 polyA; SEQ ID NO.88), Bovine Growth Hormone (BGH) polyadenylation and synthetic polyadenylation.

The term “a nucleic acid sequence encoding an RNA molecule” as described herein, refers to a nucleic acid sequence encoding an RNA molecule such as a hairpin, a double stranded RNA (dsRNA), small interfering RNA (siRNA), and microRNA (miRNA). Preferably, said nucleic acid sequence encodes a microRNA based on the miR451 scaffold. Preferably, said nucleic acid sequence comprises SEQ ID NO 124. Said RNA molecule can be used in reducing and/or knocking down the transcripts of the ANGPTL3 gene.

The term “inverted terminal repeats (ITRs)”, as described herein, refers to the sequences at the 5′ and 3′ end of said DNA expression cassette, as described above, which function in cis as origins of DNA replication and as packaging signals for the virus. Said ITRs are preferably selected from a group consisting of adeno-associated virus (AAV) ITR sequences. More preferably, said ITRs sequences are both AAV1, both AAV2, both AAV5, both AAV6, or both AAV5 ITRs sequences. Also, more preferably, said ITR sequence at the 5′ end of said DNA expression cassette differs from said ITR sequence at the 3′ of said DNA expression cassette, and said ITR sequence is selected from the AAV1, AAV2, AAV5, AAV6, and AAV8 ITRs sequences.

One objective of the present invention is to provide a virus vehicle which comprises said DNA expression cassette encoding said RNA molecule, as described above.

The term “gene therapy vehicle”, “virus vehicle” or “viral vehicle”, as described herein, refers to a wild-type or recombinant virus which acts as a vehicle to carry a genetic material, such as a gene of interest, a nucleic acid of interest, a vector comprising said gene of interest, or a vector comprising said nucleic acid of interest or a DNA expression cassette comprising said gene or nucleic acid encoding an RNA molecule into a target cell, organ or tissue. Suitable virus vehicles can be alphavirus, flavivirus, herpes simplex viruses (HSV), Simian Virus 40, measles viruses, rhabdoviruses, retrovirus, Newcastle disease virus (NDV), poxviruses, picornavirus, lentivirus, adenovirus or AAV. Preferably, said virus vehicle is an AAV gene therapy vehicle, and said AAV gene therapy vehicle comprising said DNA expression cassette, as described above. More preferably, said AAV gene therapy vehicle comprising said DNA expression cassette, wherein said DNA expression cassette comprises a nucleic acid sequence encoding an RNA molecule as described above, a promoter as described above, and a poly A tail as described above, and wherein each of the ends of said DNA expression cassette is flanked by an ITR sequence, as described above.

The term “AAV gene therapy vehicle”, as described herein, is an adeno-associated viral gene therapy vehicle. AAV viruses are classified into a number of clades based on the viral capsid protein (VP) sequence and antigenicity. Suitable AAV gene therapy vehicles, as described herein, comprise a capsid protein having an AAV1, AAV2, AAV3, AAV4, AAV5, AAV2/5 hybrid, AAV7, or AAV8 capsid protein sequence. Preferably, the capsid protein of said AAV gene therapy vehicle, as described above, has an AAV2, AAV2/5 hybrid, AAV3 or AAV5 capsid protein sequence. More preferably, the capsid protein of said AAV gene therapy vehicle, as described herein, is encoded by an AAV2/5 hybrid or AAV5 capsid protein sequence.

Also, a suitable AAV gene therapy vehicle, as described herein, comprises a capsid protein having the capsid protein sequence of AAV1, AAV2, AAV3, AAV4, AAV5, AAV6, AAV7, or AAV8 or newly developed AAV-like particles obtained by e.g. capsid shuffling techniques and AAV capsid libraries.

Optionally, capsid protein VP1, VP2, and/or VP3 for use in the present invention are selected from the known 42 serotypes.

Optionally, said capsid protein of said AAV gene therapy vehicle, as described herein, comprises VP1, VP2, and/or VP3. Also optionally, said capsid protein of said AAV gene therapy vehicle, as described herein, comprises VP1 and/or VP3.

Optionally, said AAV gene therapy vehicle comprises said DNA expression cassette wherein said DNA expression cassette comprises SEQ ID NO 124 encoding a RNA molecule or said DNA expression cassette encodes miR-451, and wherein said RNA molecule can target, cleave and/or knock down the transcripts of the ANGPTL3 gene, and wherein the capsid protein of said AAV gene therapy vehicle is encoded by an AAV2/5 hybrid capsid protein sequence or by an AAV5 capsid protein sequence.

One of the objectives of the present invention is to provide the use of said virus vehicle, as described above, as a medicament. Preferably, said medicament, as described herein, is used as a medicament for reducing and/or knocking down the transcripts of the ANGPTL3 gene. Preferably, said virus vehicle is said AAV gene therapy vehicle, as described above. Still preferably, said AAV gene therapy vehicle, as described above, is used as a medicament for reducing and/or inhibiting the level of cholesterol in the plasma, LDL-C level, TC level, TG level, phospholipids levels, and/or mild, moderate, and/or severe atherosclerotic lesions in a mammal, such as a human subject. Thereby, said AAV gene therapy vehicle, as described above, is used for the treatment and/or prevention of lipid and/or lipoprotein metabolic disorder. Preferably, lipid and/or lipoprotein metabolic disorders including hyperlipidemias such as familial hypercholesterolemia, LDL-hypercholesterolemia, hypertriglyceridemia, mixed hyperlipoproteinemia, and nonalcoholic steatohepatitis (NASH). Preferably, said AAV gene therapy vehicle, as described above, is used for the treatment and/or prevention of Dyslipidemia.

The term “composition”, as used herein, refers to a mixture, combination, and/or a formulation that comprises said nucleic acid as described above, or said AAV gene therapy vehicle as described above. Preferably, at least one molecule capable of reducing and/or inhibiting the cholesterol levels in plasma, LDL-C levels, and/or severe atherosclerotic lesions is further comprised in said composition.

Optionally, said composition further comprises at least one additive selected from the group consisting of an aqueous liquid, an organic solvent, a buffer and an excipient. Optionally, the aqueous liquid is water. Also optionally, said buffer is selected from a group consisting of acetate, citrate, phosphate, tris, histidine, and 4-(2-hydroxyethyl)-1-piperazineethanesulfonic acid (HEPES). Still optionally, the organic solvent is selected from a group consisting of ethanol, methanol, and dichloromethane. Still more, the excipient is a salt, sugar, cholesterol or fatty acid. Still optionally, said salt, as described above, is selected from a group consisting of sodium chloride, potassium chloride. Yet optionally, said sugar, as described above, is sucrose, mannitol, trehalose, and/or dextran.

One objective of the present invention is to provide a kit comprising said nucleic acid, as described above, said RNA molecule, as described above, said composition comprising said RNA molecule, as described above, said composition comprising said nucleic acid, as described above, or said AAV gene therapy vehicle, as described above.

For the purpose of treating and/or preventing the diseases or disorders as described above, said composition, as described above, and optionally at least one additive such as an excipient, as described above, may conveniently be combined into a kit. Thus, the term “kit” as described herein, includes at least said nucleic acid as described above, at least said RNA molecule as described above, or said AAV gene therapy vehicle, as described above, or said composition as described above, and means for retaining said nucleic acid, said AAV gene therapy vehicle, said RNA molecule, or said composition, such as a container or a bottle.

Suitably, the composition comprising said RNA molecule, as described above and herein, and/or said composition comprising said nucleic acid, as described above and herein, is retained in a container comprised in the kit. Medical practitioners and patients can readily follow the labels and/or the instructions to apply said composition and/or said AAV gene therapy vehicle, as described above, on a mammal, such as a human subject.

### Examples of the Present Invention

The main indication for dyslipidemia treatment is prevention of atherosclerotic cardiovascular diseases. Patients with lipid disorders should adopt a healthy lifestyle (heart healthy diet, regular exercise, avoidance of tobacco, and maintaining a healthy weight) regardless of whether drug therapy is being prescribed. Statins are the preferred drugs to lower lipids. Additional drugs have emerged as agents to decrease lipids, such as ezetimibe that is a PCSK9 inhibitor. However, these drugs alone do not decrease the risk for atherosclerotic disease. Pharmacologic interventions that are not recommended for primary prevention include fibrates, bile acid-binding resins, omega-3 fatty acid supplements, plant sterols or stanols, and niacin (Kopin, L. and Lowenstein, C. 2017). Medical procedures (such as lipoprotein apheresis) that lower cholesterol levels are reserved for people with very high levels of LDL-C that do not respond to diet and lipid-lowering drugs. Such people include those with familial hypercholesterolemia (https://www.ncbi.nlm.nih.gov/books/NBK425700/). At present, few efficacious drugs are available that can reduce severely elevated remnant lipoproteins, triglyceride-rich lipoproteins and/or Lp(a) levels. These lipoproteins can be reduced using novel gene silencing approaches such as ASO inhibition and small interfering RNA (siRNA) technology by targeting proteins that have an important role in lipoprotein production or removal (Nordestgaard, B. G. et al., 2018). Angiopoietin-like 3 (ANGPTL3) protein represents one of central regulators of TG and triglyceride-rich lipoproteins (TRL) metabolism and are considered attractive therapeutic targets (Olkkonen, V. M. et al., 2018). Previous studies report that targeting ANGPTL3, in which loss-of-function mutations are naturally occurring, is safe. Individuals with no or reduced circulating ANGPTL3 protein had no perturbation in the whole-body cholesterol homeostasis and was not associated with pathological conditions (Minicocci, I. et al., 2012).

The present inventors now sought to provide for a gene therapy approach for the treatment of dyslipidemia that is both safe and effective for human use by silencing human ANGPTL3 gene expression using microRNA constructs delivered with adeno-associated viral vector of serotype 5 (AAV5). Conserved target regions of ANGPTL3 across non-human primates (NHPs), humans and ideally rodents, are targeted using the microRNA constructs (miANGs). Eighteen constructs were generated and screened for their ability to knockdown a luciferase reporter construct and endogenous mRNA expression in human liver cells. Three potent silencing constructs were selected for further testing using AAV vectors in rodents and a dyslipidemic mouse model. Upon successful proof of concept (PoC) in small animals, AAVs were tested in combination with statins in dyslipidemic mice and NHPs.

Design of Therapeutic miRNAs Targeting Angiopoietin-Like Protein 3 (ANGPTL3)

### ANGPTL3 RNA Target Sequence Analysis and Guide Strand Design

The miANGs, miRNA guide strands targeting conserved RNA sequences of the ANGPTL3 genes throughout different species were designed. The full length of the ANGPTL3 mRNA sequences of selected species (Homo sapiens, the NCBI accession number NM_014495.4, SEQ ID NO.2; Macaca fascicularis, the NCBI accession number XM_005543185.2, SEQ ID NO.89; Mus musculus, the NCBI accession number NM_013913.4, SEQ ID NO.90; Rattus norvegicus, the NCBI accession number NM_001025065.1, SEQ ID NO.91) were aligned. Multiple Sequence Comparison by Log-Expectatio (MUSCLE) alignment tool was used to perform the alignment of the ANGPTL3 mRNA sequences with default settings (https://www.ebi.ac.uk/Tools/msa/muscle/). Four conserved sequences (SEQ ID NOs. 3-6) of the ANGPTL3 mRNA sequences in human, monkey, mouse and rat were identified and used for the design of 14 miRNAs (SEQ ID NOs. 8-21). Each of the conserved sequences was used to generate a number of different guide strands having 22 nucleotides (nts) with the “tiling strategy”. The strategy consisted in designing overlapping 22 nt guides to fully cover a conserved sequence larger than 22 nts or, to extend the conserved sequence in 5′ or 3′ direction when the conserved sequence was shorter than 19 nts. Seven guides targeting ANGPTL3 (named miANG1-miANG7, SEQ ID NOs. 8-14) were designed on the first conserved region (NM_014495.4: position 139-166 nt) located in exon 1. Three guides targeting ANGPTL3 (named miANG8-miANG10, SEQ ID NOs. 15-17) were designed on the second conserved region (NM_014495.4: position 267-292 nt) located in exon 1. Two guides targeting ANGPTL3 (named miANG11 and miANG12, SEQ ID NOs. 18 and 19) were designed on the third conserved region (NM_014495.4: position 706-728 nt) located in exon 3. Two guides targeting ANGPTL3 (named miANG13 and miANG14, SEQ ID NOs. 20 and 21) were designed on the fourth conserved region (NM_014495.4: position 885-907 nt) located in exon 5. Four guides were designed by overlapping the ASO IONIS-ANGPLT3Rx developed by Ionis (SEQ ID NOs. 22 through 25). IONIS-ANGPTL3Rx (SEQ ID NO. 7) is a second-generation 2′-O-methoxyethyl (2′-MOE) chimeric antisense oligonucleotide drug targeting the ANGPTL3 mRNA sequence consisting of the nucleotide sequence 5′-GGACATTGCCAGTAATCGCA-3′ (Graham, M. J. et. al., 2017). The nucleotide sequence of IONIS-ANGPTL3Rx is complementary to a 20 nts sequence within exon 6 of the ANGPTL3 mRNA coding sequence at position 1136-1155 of the sequence with the NCBI NM_014495.4. The newly designed sequences of miANG15, miANG16 and miANG17 (SEQ ID NOs. 22-24) were identical to ASO IONIS-ANGPTL3Rx with two more nts at 5′ of the sequence, one more nucleotide at 5′ and 3′ of the sequence, and two more nts at 3′ of the sequence, respectively (NM_014495.4: position 1134-1157). miANG18 (SEQ ID NO. 25) has 17 nts sequence overlapping with ASO IONIS-ANGPTL3Rx (NM_014495.4: position 1139-1155) and four more nucleotides at 3′ of the sequence (NM_014495.4: position 1156-1160) To generate a negative control the miANG6 and guides were scrambled using the GenScript software (https://www.genscript.com/tools/create-scrambled-sequence). The scramble guides were named miANG-SCR1 and miANG-SCR2 (SEQ ID NOs.92 and 93).

### DNA Constructs

The miANGs and the miANG-SCR controls were embedded in the human pre-miR-451 scaffold (FIG. 2.A), flanked by 90 nts of 5′ and 3′ flanking regions, AscI and NotI restriction sites were added respectively at the 5′ and 3′ and the complete sequence was gene synthesized (GeneArt, Thermo Fisher Scientific). The pri-miANG cassettes were expressed from the HCR-hAAT promoter (the apolipoprotein E locus control region, human alpha1-antitrypsin, SEQ ID NO.94) and terminated by the simian virus 40 polyadenylation (SV40 polyA, SEQ ID NO.88) signal (FIG. 2.B). Two luciferase reporters LucANG-A (SEQ ID NO.95) and LucANG-B (SEQ ID NO.96) were generated by respectively combining the fragments of the exons 1, 3, 4, (NM_014495.4: positions 128-177, 255-304, 692-741, 871-920; FIG. 2.C) and exon 6 and 7 (NM_014495.4: position 1121-1170; FIG. 2.C). Flanking regions at the 5′ and 3′ were included with AscI and NotI restriction sites. The complete sequences as well the cloning in the 3′UTR of the Renilla luciferase (RL) gene of the psiCHECK-2 vector (Promega, Thermo Fisher Scientific) were synthesized and cloned by GeneArt (Invitrogen, Thermo Fisher Scientific). The secondary structure of the RNA transcripts was predicted using the mfold program (http://mfold.rna.albany.edu/?q=mfold, Zucker 2013).

### Materials and Methods

In Vitro Experiments

### Transfection Assays and Cells

The human hepatocyte derived cellular carcinoma Huh-7 cells were maintained in Dulbecco's modified Eagle's medium (Thermo Fisher Scientific) containing 10% fetal calf serum (Greiner, Kremsmünster), at 37° C. and 5% CO₂. For luciferase assays and small RNA NGS, cells were seeded in 24-well plates at a density of 1E+05 cells per well in Dulbecco's modified Eagle's medium (Thermo Fisher Scientific) one day prior transfection. Transfections were performed with Lipofectamine 3000 reagent (Thermo Fisher Scientific) according to the manufacturer's instructions.

### Dual Reporter Luciferase Assay

Huh-7 cells were cotransfected in triplicate with miANG expression constructs and luciferase reporters that contain both the RL gene fused to ANGPTL3 target sequences and the Firefly luciferase (FL) gene. pBluescript was added to transfect equal amounts of DNA. Transfected cells were assayed at 48 hours post-transfection in 100 μl 1× passive lysis buffer (Promega, Thermo Fisher Scientific) by gentle rocking for 15 minutes at room temperature. The cell lysates were centrifuged for 5 minutes at 4,000 rpm and 10 μl of the supernatant was used to measure FL and RL activities with the Dual-Luciferase Reporter Assay System (Promega, Thermo Fisher Scientific). Relative luciferase activity was calculated as the ratio between RL and FL activities.

### RNA Isolation and Next-Generation Sequencing (NGS)

Huh-7 cells were transfected with 250 ng or 400 ng of miANG5, miANG10 and miANG13 constructs using Lipofectamine 3000 reagent (Thermo Fisher Scientific) and total RNA was isolated from cells 48 hours post-transfection using TRIzol® Reagent (Thermo Fisher Scientific) and Direct-zol RNA Miniprep (Zymo Research) according to the manufacturer's protocol. RNA samples were treated with dsDNase from Thermo Fisher Scientific according to manufacturer's instructions. For sequencing, total RNA samples from miANG5, miANG10, miANG13 and untransfected Huh-7 was sent out for small RNA sequencing (BaseClear B.V.). Small RNA sequencing libraries for the Illumina platform were prepared and sequenced at BaseClear B.V.

### NGS Data Analysis

Analysis of the miRNA expression and processing in transfected Huh-7 cells was performed using CLC Genomics Workbench 10. The obtained reads were adaptor-trimmed. The custom adapter sequence used for trimming in the plus strand was TGGAATTCTCGGGTGCCAAGG, and that in the minus strand was CCTTGGCACCCGAGAATTCCA. A second trimming was performed, and 4 nts were removed from the 5′ and 3′ of each read. All reads containing ambiguity N symbols, reads shorter than 15 nts or longer than 70 nts were excluded. Next, the obtained unique small RNA reads were annotated using miRNA human database (miRBase) and aligned to the references sequences of the pri-miANG constructs. The percentage of expression of miANG5, miANG10 and miANG13 in the total pool of endogenous miRNAs was calculated by the software CLC Genomics Workbench 10 during the annotation process. To investigate the processing of miANG5, miANG10 and miANG13, length and percentage of each mature miRNA species were assessed by considering the top 20 most abundant annotations (set to 100%) against the appropriate pri-miANG sequence (SED ID. NOs.66, 77 and 75).

Measurement of Endogenous Huh-7 ANGPTL3 mRNA Knockdown

RT-QPCR was performed to confirm miRNA expression by knockdown of endogenous ANGPTL3 mRNA. Huh-7 cells were transfected with miANG5, miANG10, miANG13, miANG-SCR1 and miANG-SCR2 constructs. Two days after transfection, the medium was refreshed. Cell monolayers were harvested with TRIzol® Reagent (Thermo Fisher Scientific) 48 hours after transfection and RNA was isolated using Direct-zol RNA Miniprep (Zymo Research) according to manufacturer's instructions. DNase treatment and cDNA synthesis were performed by using Ambion® TURBO DNA-free™ DNase Treatment (ThermoFisher Scientific) and Maxima First Strand cDNA Synthesis Kit (Thermo Fisher Scientific) according to manufacturer's instructions. QPCR was performed with TaqMan ready-to-use primer-probe (Thermo Fisher Scientific) from Gene Expression Assay (Thermo Fisher Scientific): ANGPTL3 (Assay ID: Hs00205581_m1, Thermo Fisher Scientific) and β-actin (ACTB) as housekeeping gene (Assay ID: Hs01060665_g1, Thermo Fisher Scientific). Relative gene expression data were obtained normalizing ANGPTL3 data with human ACTB as reference gene. Results are shown relative to the miANG-SCR1 sample set to 100%.

### DNA Constructs for Baculovirus Seed Generation

The expression cassettes were incorporated in a plasmid encoding the AAV ITRs. The expression cassettes comprising a promoter sequence driving the expression of miRNA targeting ANGPLT3. Expression cassettes used in the examples comprise e.g. promoter sequences such as listed in SEQ ID NO.94 representing the apolipoprotein E locus control region (HCR), human alpha1-antitrypsin (hAAT) promoter (HRC-hAAT), combined with miRNA encoding sequences such as listed e.g. in SEQ ID NO.66 (pri-miANG5). Exemplary expression cassettes as used in the studies being listed in SEQ ID NO.97 (hAAT-pri-miANG5). An example of a representative viral vector genome is listed in SEQ ID NO.98, which comprises the hAAT-pri-miANG5 expression cassette.

### AAV5 Vectors

Recombinant AAV5 (SEQ ID NO.99) harboring the expression cassettes were produced by infecting SF+ insect cells (Protein Sciences Corporation, Meriden, Conn., USA) with two Baculoviruses, encoding Rep, Cap and Transgene. Following standard protein purification procedures on a fast protein liquid chromatography system (AKTA Explorer, GE 30 Healthcare) using AVB sepharose (GE Healthcare) the titer of the purified AAV was determined using QPCR.

### AAV5 Transduction of Huh-7 Cells

The human hepatocyte derived cellular carcinoma Huh-7 cells were maintained in Dulbecco's modified Eagle's medium (Thermo Fisher Scientific) containing 10% fetal calf serum (Greiner), at 37° C. and 5% CO₂. For transduction assays, cells were seeded in 24-well plates at a density of 1E+05 cells per well in Dulbecco's modified Eagle's medium (Thermo Fisher Scientific) one day prior transduction. Cells were transduced with 100 μL of AAV5 vectors at a multiplicity of infection (MOI) of 1E+05, 1E+06 and 1E+07 genome copies (gc) per cell in triplicate. Three days post-transduction, the monolayers were harvested in 200 μl RTL plus buffer (AllPrep DNA/RNA Mini Kit, Qiagen). Three wells belonging to the same condition were pooled for DNA extraction.

Vector DNA Isolation and Quantification from Cells

DNA extraction was performed using AllPrep DNA/RNA Mini Kit (Qiagen) and following manufacturer's instructions. Vector genome copies were quantified by using TaqMan QPCR assay (Thermo Fisher scientific) (SEQ ID NO.100 through 102) and ACTB SybrGreen assay was used as loading control gene (SEQ ID NO.103 and SEQ ID NO.104).

Animals Studies

### Mouse Studies

To study ANGPTL3 mRNA and protein lowering in C57BL/6 mice upon IV injection of AAV5 vectors, 6-8 weeks old male wild type C57/BL6JRj mice (n=6) received 1E+13, 5E+13 and 2.5E+14 gc/kg of AAV5-miANG5 and 2.5E+14 gc/kg of AAV5-miANG-SCR1 vectors in their tail vein. At 2, 4, 6 and 8 weeks post-treatment, blood samples were taken to determine the ANGPTL3 protein level in the plasma, alanine aminotransferase (ALT) and aspartate aminotransferase (AST) levels, total cholesterol (TC) and TG levels. At week 8 animals were sacrificed, livers were taken from the mice to extract DNA for vector genome quantification and RNA for murine Angptl3 mRNA expression and miANG5 quantification.

To examine the effect of AAV-mediated gene silencing of ANGPTL3 on plasma lipid metabolism and development of atherosclerosis, APOE*3-Leiden.CETP mice were used (TNO, the Netherlands). Eighty-two APOE*3-Leiden.CETP transgenic female mice, of approximately 8-10 weeks old, were put on a Western-type diet (WTD) with 0.15% cholesterol and 15% saturated fat. After a 3 week run-in period, 17 low-responder mice were removed from the study and the remaining 65 mice were subdivided in 4 groups; n=15 for all groups except group 1 that had 20 mice, matched for age, body weight, plasma total TC, and TG after 4 hours fasting. In week 0, the mice received an IV tail vein injection of AAV5 vector at a dose of 5E+13 gc/kg of AAV5-miANG-SCR1 (Group 2) or AAV5-miANG5 (Group 3) or AAV5-miANG13 (Group 4). A control group (Group 1) received the formulation buffer (vehicle) only. Body weight (individual) and food intake (per cage) were determined in week 0, 1, 2, 4, 6, 8, 10, 12, 14, and 16. Plasma total cholesterol and triglycerides were measured in week 0, 2, 4, 6, 8, 10, 12, 14, and 16. Plasma ALT and AST as markers for liver injury were measured in week 0, 1, 4, 8, 12, and 16 using group pooled plasma samples. In week 0, 4, 8, 12, and 16, lipoprotein profiles were measured using group pooled plasma samples. In week 12, 5 mice in Group 1 were sacrificed via CO₂ asphyxiation, non-fasted, to evaluate atherosclerosis development in the aortic root. Based on the cholesterol exposure and atherosclerosis development in the mice selected for the pilot sacrifice, the cholesterol exposure of the rest of the mice in the control group and the curve showing the relationship between lesion area and cholesterol exposure the study was prolonged to a total of 16 weeks after AAV injections. In week 16 after AAV injection, mice were sacrificed via CO₂ asphyxiation, non-fasted. EDTA-plasma was obtained via heart puncture. Heart, aorta, liver, and spleen tissue were collected. Livers were used to extract DNA for vector genome quantification and RNA for murine Angplt3 expression, and miANG5 levels.

A study was carried out to examine the effect of AAV-mediated gene silencing of ANGPTL3 on development of atherosclerosis, alone and in combination with atorvastatin treatment in APOE*3-Leiden.CETP mice (TNO, the Netherlands). Hundred approximately 8-12 old weeks old female APOE*3 Leiden.CETP mice were put on a Western-type diet (WTD) with 0.15% cholesterol and 15% saturated fat. After 3-weeks run-in period 20 low-responder mice are removed from the study and the remaining 80 mice are sub-divided into one control group of mice (group 1) and 4 AAV treatment groups. The treatment groups are matched for age, body weight, plasma cholesterol and triglycerides after 4 hr fasting. The animals are dosed with 1E+14 gc/kg of AAV vectors via IV tail vein injection. Atorvastatin is administered by diet admix at a concentration of 0.0035% (w/w) (approximately 3.5 mg/kg body weight/day). The groups are as follows: group 1: formulation buffer (vehicle) n=20, group 2: AAV-miANG-SCR n=15, group 3: AAV-miANG5 n=15, group 4: AAV-miANG-SCR+Atorvastatin n=15 and group 5: AAV-miANG5+Atorvastatin. Body weight (individual) and food intake (per cage) are determined in week 0, 1, 2, 4, 6, 8, 10, 12, 14, and 16. Plasma total cholesterol and triglycerides are measured in week 0, 2, 4, 6, 8, 10, 12, 14, and 16. Plasma ALT and AST as markers for liver injury are measured in week 0, 1, 4, 8, 12, and 16 using group pooled plasma samples. In week 0, 4, 8, 12, and 16, lipoprotein profiles are measured using group pooled plasma samples. Pools include samples of mice with confirmed effects on plasma cholesterol and/or triglycerides to rule out inclusion of mice that do not receive a correct AAV dose due to unsuccessful injection. In week 12, 5 mice in group 1 are sacrificed via CO₂ asphyxiation, non-fasted, to evaluate atherosclerosis development in the aortic root. The cholesterol exposure (plasma cholesterol concentration x duration) of approximately 280 mM weeks, resulting in an expected lesion area of approximately 160,000 μm2 (data based on a curve showing the relationship between lesion area and cholesterol exposure, made on the basis of previous studies in female E3L.CETP transgenic mice performed by TNO) is to be observed. Based on the cholesterol exposure and atherosclerosis development in the mice selected for the pilot sacrifice, the cholesterol exposure of the rest of the mice in the control group and the curve showing the relationship between lesion area and cholesterol exposure, a prediction on the expected atherosclerosis development of the control group is made. When the expected atherosclerosis development of the control group is <100,000 μm2, the study plan can be adjusted, after consultation with the Sponsor, to prolong the study for 2 weeks to a total of 18 weeks after AAV injections. If this is not the case, the remaining mice are sacrificed in week 16. In week 16 or 18 after AAV injection, mice are sacrificed via CO₂ asphyxiation, non-fasted. EDTA-plasma is obtained via heart puncture. Heart, aorta, liver, kidney, and spleen tissues are collected.

### AAV Testing in Diet-Induced Dyslipidemia in Cynomolgus Monkeys

The in-life experimental procedures were in accordance with the Animal Welfare Act, the Guide for the Care and Use of Laboratory Animals, and the Office of Laboratory Animal Welfare Animals are housed individually in stainless steel cages (except during periods of commingling). Animals are provided either Teklad TD 110084 or LabDiet® 5AVO (LabDiet® 5040 w/ Hi Fructose, Fat, and 0.25% Cholesterol) or LabDiet® 9GA6 (LabDiet® 5040 w/ Hi Fructose, Fat, and 0.05% Cholesterol) (LabDiet, U.S.A.) at least 2 times daily for a period of at least 8 weeks prior to the AAV injection. Only animals with measured triglyceride levels of at least 85 mg/dL are included in the study. In addition, the animals are prescreened for their AAV5 neutralizing antibody titer, sequence of the target region (liver biopsy), plasma lipid profile, and diet preference to assign the animals to the dosing groups.

Male Cynomolgus Macaques (Macaca fascicularis; n=3 per group) received a single intravenous administration of the AAV encoding the miRNA targeting ANGPTL3 at a dose of 1E+14 gc/kg or the formulation buffer. Blood is collected prior to the AAV-injection and throughout the duration of the study to detect ANGPTL3 and miRNA levels, lipid profiles, vector clearance, clinical chemistry and hematology markers. At day 57 to 84 post-AAV injection, the animals received Simvastatin daily at a dose of 20 mg/kg to study the effect of the gene therapy in combination with Simvastatin. Prior to the diet, once after the diet and at day 29, 57, 85 and 120 post-AAV dosing, the animals received a surgical liver biopsy under anesthesia and analgesia. At day 141 post-treatment, the animals are sacrificed and examined. A number of organs including adrenals, brain, heart, kidney, liver, spleen, testis, lungs and subcutaneous abdominal white fat are collected.

Vector DNA, mRNA and miANG5 Quantification in Mouse and Monkey Liver

DNA from the livers is extracted using the DNeasy® Blood and Tissue kit (Qiagen) according to the supplier's protocol. Vector genome copies are quantified as described in the paragraph “Vector DNA isolation and quantification from cells”. ANGPTL3 mRNA levels in livers are determined as previously described in “Measurement of endogenous Huh-7 ANGPTL3 mRNA knockdown” paragraph. To quantify miANG5, RNA from livers is reverse transcribed using the TaqMan MicroRNA Reverse Transcription kit (Thermo Fisher Scientific) with a reverse transcription primer specific for the 24 nts (primer target sequence SEQ ID NO.105) or 23 nts (variant T) processed miANG5 (primer target sequence SEQ ID NO.125). Two custom TaqMan QPCR small RNA assay (Thermo Fischer Scientific) are performed to measure the most abundant miANG5 species of 24 nts (Assay ID CTFVKZT, Rack ID, SEQ ID NO.106) or 23 nts (variant T) in length (Assay ID CTGZFJPSEQ ID NO.126). A serial dilution of the synthetic RNA oligo is used as standard to calculate the amount of miANG5 24 nts (SEQ ID NO.107) or 23 nt (variant T; SEQ ID NO.127) molecules/cell per liver sample (Integrated DNA Technologies).

### Mouse ANGPTL3 ELISA

Murine ANGPTL3 protein in the plasma was determined with a commercially available Enzyme-Linked Immunosorbent Assay (ELISA) kit RAB0756 (Sigma-Aldrich) according to the manufacturer's instructions. Plasma samples were diluted in provided dilution buffer to obtain an optical density (O.D.) value which fits in the reference standard curve and each plasma sample was measured in duplicate. Reference curve is generated by preparing a serial dilution of a standard included in the ELISA kit according to the supplier's protocol.

### Measurement of Lipids Profile in Mouse Plasma

To measure total cholesterol levels in murine plasma, the commercially available Amplex Red Cholesterol Assay Kit (Cat. No. A12216, Thermo Fisher Scientific) was used according to manufacturer's instructions. Triglycerides levels in murine plasma were determined using the Triglyceride Quantification Kit (Cat. No. MAK266, Sigma-Aldrich) according to the manufacturer's instructions.

### ALT and AST Activity Assay

Alanine Aminotransferase (ALT) and aspartate aminotransferase (AST) activity assay was performed in murine plasma samples to detect hepatocellular injury. Two commercially available kits, Aspartate Aminotransferase Activity Assay Kit (Cat. No. MAK055, Sigma-Aldrich) and Alanine Aminotransferase Activity Assay Kit (Cat. No. MAK052, Sigma-Aldrich) were used according to manufacturer's instructions.

### Results

In Vitro Experiments Results

In Vitro Silencing Efficacy of Artificial miANG Constructs

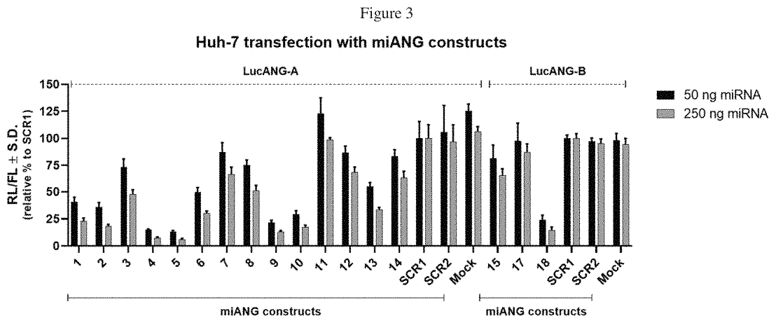

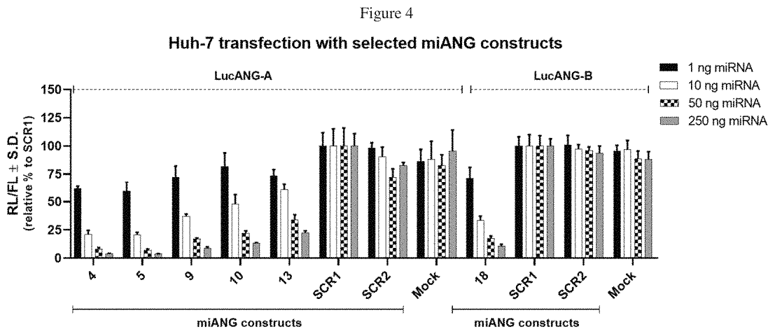

To evaluate the miANG knockdown efficacy of the miANG constructs in vitro, Huh-7 cells were co-transfected with Renilla luciferase reporters encoding the ANG target sequences and said miANG constructs. The Firefly luciferase (FL) gene was expressed from the same reporter vector and served as an internal control to correct for transfection efficiency. In the first screening Huh-7 cells were co-transfected with 50 or 250 ng of each of the miANG constructs, miANG-SCR1, miANG-SCR2 and pBlueScript (pBS) and 50 ng of LucANG-A or LucANG-B. From miANG1-miANG14 constructs designed to target ANGPTL3 exon 1, 3 and 5, miANG1, miANG2, miANG3, miANG6, miANG8 and miANG13 induced mild luciferase knockdown between 25-65%. miANG4, miANG5, miANG9, miANG10 and miANG18 (targeting ANGPTL3 exon 6) construct was highly effective and induced more than 70% inhibition of the ANGPTL3 luciferase reporter plasmid (FIG. 3). To further determine the potency, a number of miANG constructs were selected for titration experiments (4, 5, 9, 10, 13 and 18). The constructs were co-transfected in Huh-7 cells in different concentrations; 1, 10, 50 or 250 ng with 10 ng ofANGPTL3 luciferase reporter plasmid (FIG. 4). The lowest miRNA concentration tested of 1 ng (ratio luciferase:miRNA is 10:1) was able to elicit approximately a knockdown between 20% and 40%. The knockdown measured at increased miRNA concentration was respectively 20-75% with 10 ng miRNA (ratio luciferase:miRNA is 1:1), 60-90% with 50 ng miRNA (ratio luciferase:miRNA is 1:5) and 70-96% with 250 ng miRNA (ratio luciferase:miRNA is 1:25). The most potent constructs were miANG5 and miANG4, both targeting a similar region of ANGPTL3 exon 1, followed by miANG18, miANG10 and miANG13. miANG5, and miANG10 and miANG13 respectively targeting ANGPTL3 exon 3 and exon 5, were the candidates for a further in vitro testing, NGS analysis, baculovirus generation and AAV5 production. Although miANG18 was a potent candidate based on its Luc knockdown potential, it was not further tested because its specificity is restricted to human and monkey species and not rodent species.

### Lowering of Endogenous ANGPTL3 Expression in Transfected Cells

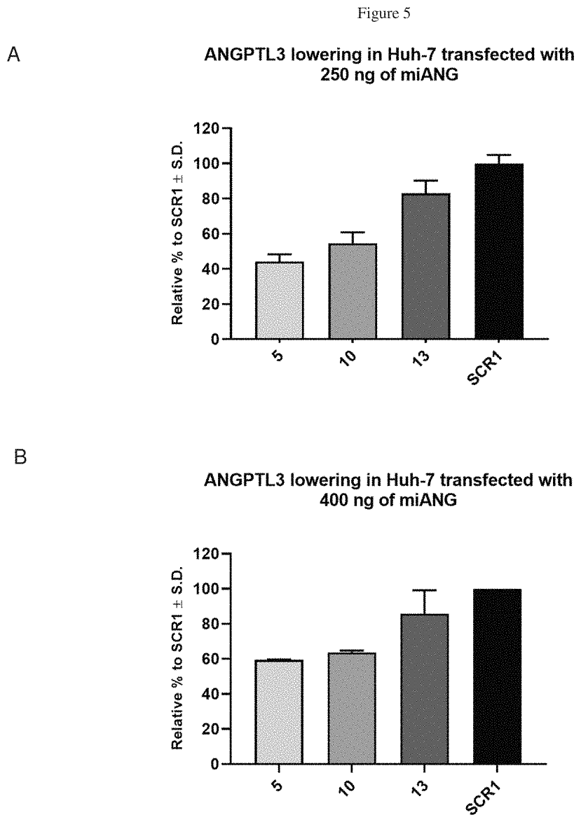

miANG5, miANG10 and miANG13 constructs were chosen for testing the knockdown of ANGPTL3 mRNA expression in cells. The knockdown of the endogenous ANGPTL3 gene expression in Huh-7 cells was confirmed by RT-QPCR on transfected cells. Transfection of 250 ng of miRNA plasmid resulted in a decrease of ANGPTL3 mRNA expression of ˜60% by miANG5, followed by miANG10 and miANG13 with a knockdown of ˜50 and 20%, respectively (FIG. 5.A). The transfection of 400 ng of miRNA plasmid showed similar results as the transfection using 250 ng construct (FIG. 5.B).

Expression Levels of miRNAs in Transfected Cells (NGS Data)

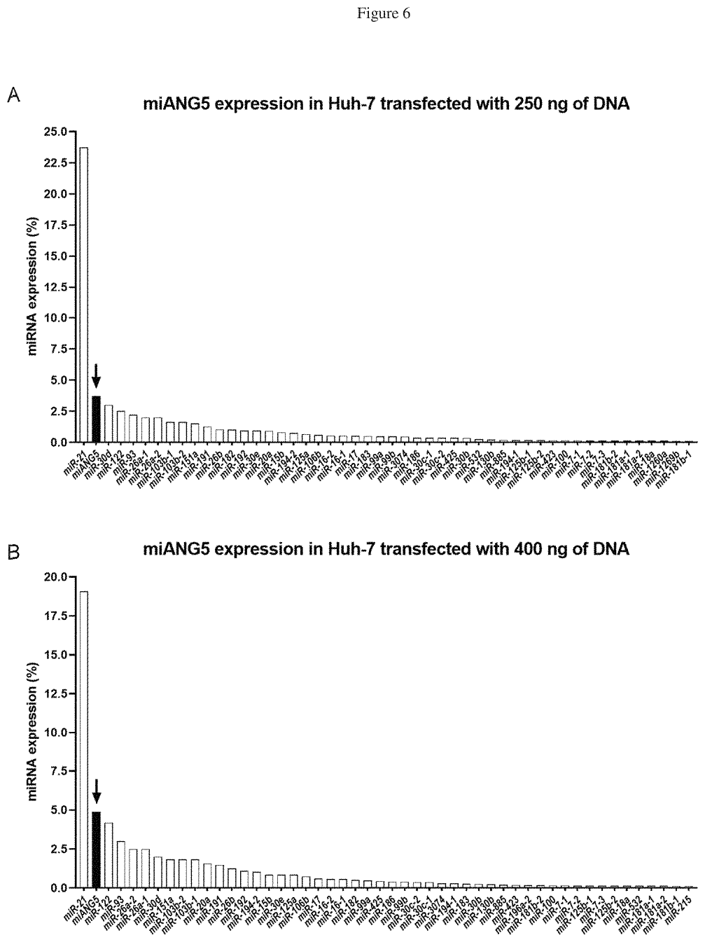

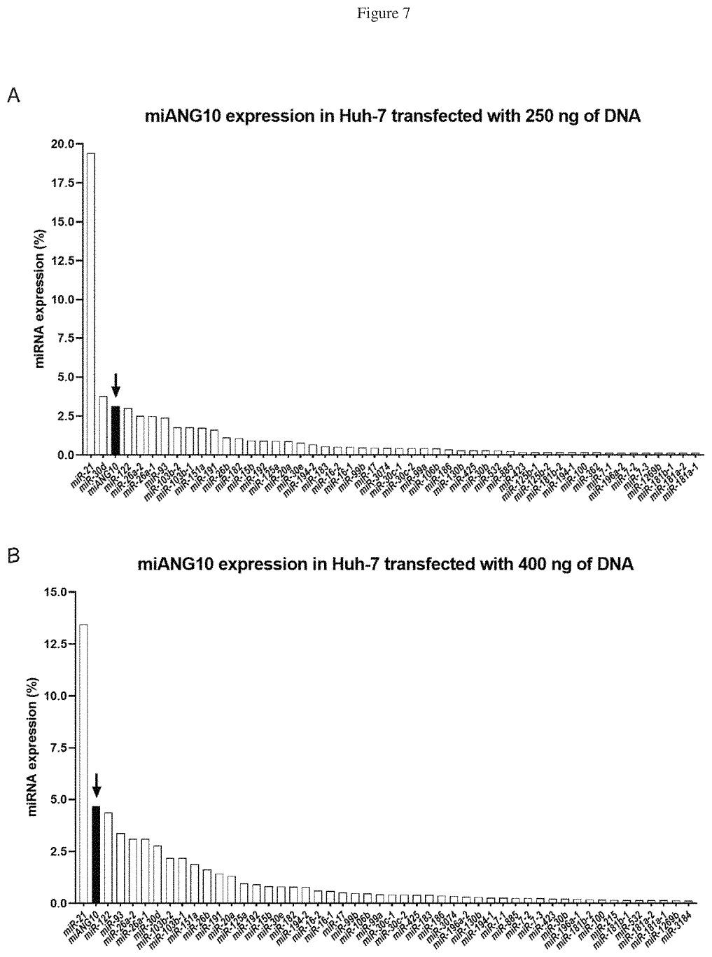

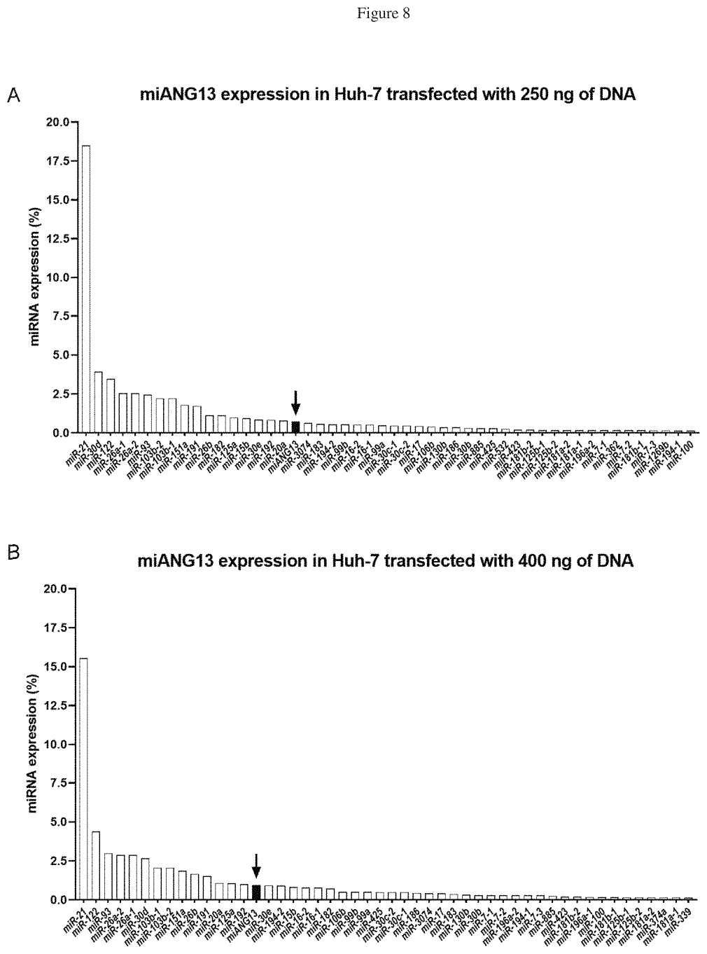

The expression level of the mature miRNAs was quantified based on the number of the total reads annotated by using miRBase and the pre-miRNA sequence of interest (SED ID. NOs.66, 71 and 74). FIGS. 6.A and B showed the expression levels of the top 50 most expressed miRNAs in Huh-7 transfected with 250 or 400 ng of miANG5. miANG5 was the second most abundant mature miRNA found in Huh-7. All the processed forms of miANG5 counted for 3.7% (250 ng transfection) and 4.9% (400 ng transfection) of the total annotated reads. Similar to miANG5, miANG10 was one of the most abundant miRNAs in transfected Huh-7, the third most expressed when 250 ng of DNA were transfected and an expression level of 3.1% (FIG. 7.A) and the second most expressed miRNA when using 400 ng of DNA reaching the expression level of 4.7%. (FIG. 7.B). Results from miANG13 abundancy are shown in FIGS. 8.A and B. All the processed forms of miANG13 counted for 0.7% (250 ng transfection) and 1% (400 ng transfection) of the total annotated reads. In both set of experiments the results showed that the expression levels of the transfected miRNAs are not exceeding those of the endogenous Huh-7 miRNAs and at higher DNA concentration corresponded increased miRNA expression levels.

Processing of miANG Constructs Upon Transfection in Cells (NGS Data)

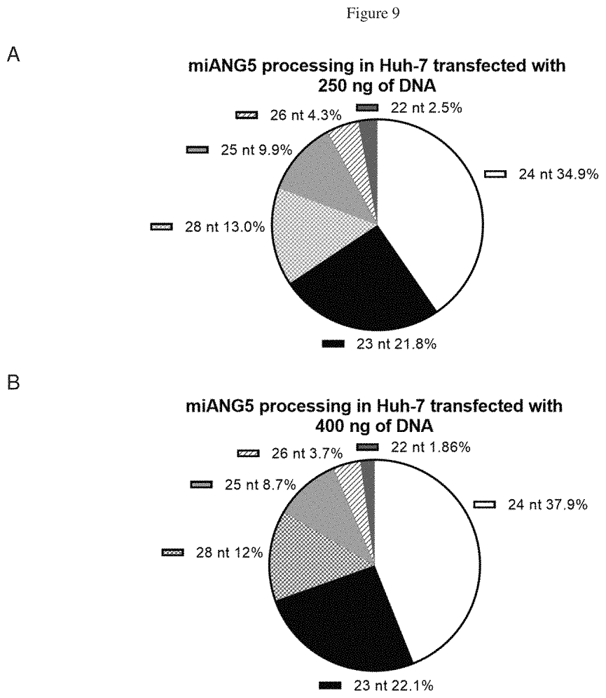

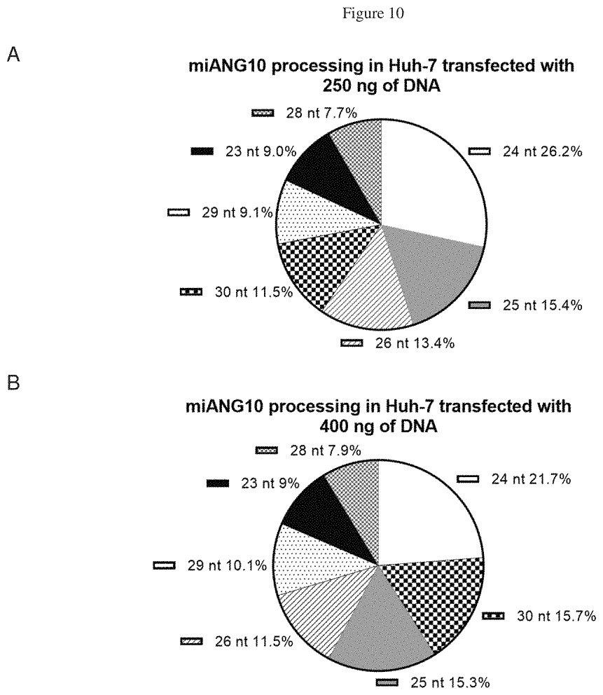

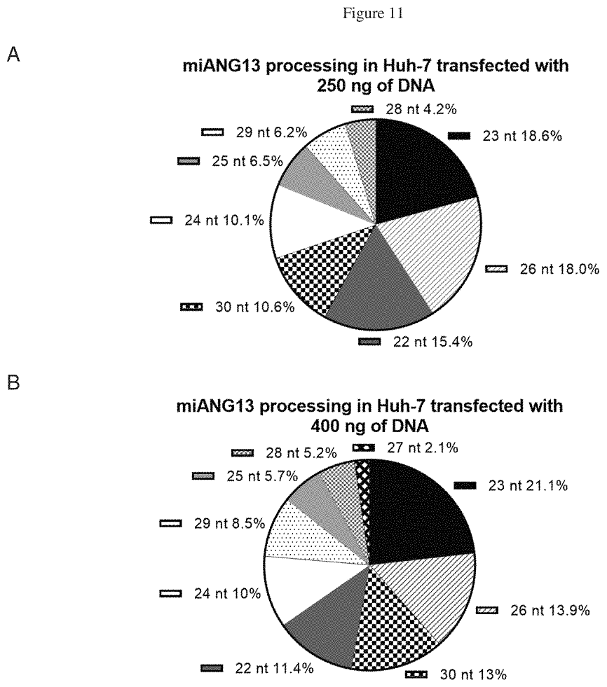

The miRNAs processing was also investigated by alignment of the reads to the pre-miRNA sequences SED ID. NOs.66, 71 and 74. The top 20 most abundant mature forms obtained from the annotation process were considered for graphical purposes and set to 100%. No mismatches with the reference sequences were allowed and the reads represented with less than 2% are not shown. Independently from the amount of miRNA plasmid (250 ng or 400 ng) transfected in Huh-7 cells, the length of the most abundant form for miANG5 was 24 nts (FIGS. 9.A and B), for miANG10 was 24 nts (FIGS. 10.A and B) and for miANG13 was 23 nts (FIGS. 11.A and B). Observed mismatches with the reference sequence consisted in sequence modification at the 3′ in which adenine or thymine (uracil) was added to the mature guide sequences. For all the constructs none of these variants reached more than the 2% threshold set for analysis. This is in accordance with previously published data on 3′ end editing events in various cell lines and tissues (Landgraf, P. et al., 2007). However, the exact roles of mono-uridylation and mono-adenylation still needs to be determined.

### AAV5-miANG Transduction in Cells

To investigate the ability of obtained AAV5-miANG5, AAV5-miANG10, AAV5-miANG13 and AAV5-miANG-SCR1 to transduce and deliver the packaged expression cassette, Huh-7 cells were transduced (n=1) at a Multiplicity of Infection (MOI) of 1E+07 (tested only for miANG5 and miANG-SCR), 1E+06 and 1E+05 gc/cell. Vector DNA was quantified by QPCR. The results showed a dose-dependent increase in detected vector genome DNA copies at higher MOIs. (Table 6).

TABLE 6
Vector genome DNA from transduced Huh-7 cells
with miANG5, miANG10, miANG13 and miANG-SCR1

| AAV5 vector | MOI 1E+05 | MOI 1E+06 | MOI 1E+07 |
|---|---|---|---|
| encoding: | gc/μg DNA | gc/μg DNA | gc/μg DNA |
| miANG5 | 1.76E+07 | 7.35E+08 | 4.5E+09 |
| miANG10 | 2.15E+05 | 2.19E+06 | n.a.* |
| miANG13 | 6.2E+04 | 1.81E+06 | n.a.* |
| miANG-SCR1 | 5.18E+07 | 3.92E+09 | 1.02E+10 |

*not analyzed

In Vivo Experiments Results

Processing of miANG5 in Livers of APOE*3-Leiden.CETP Transgenic Mice

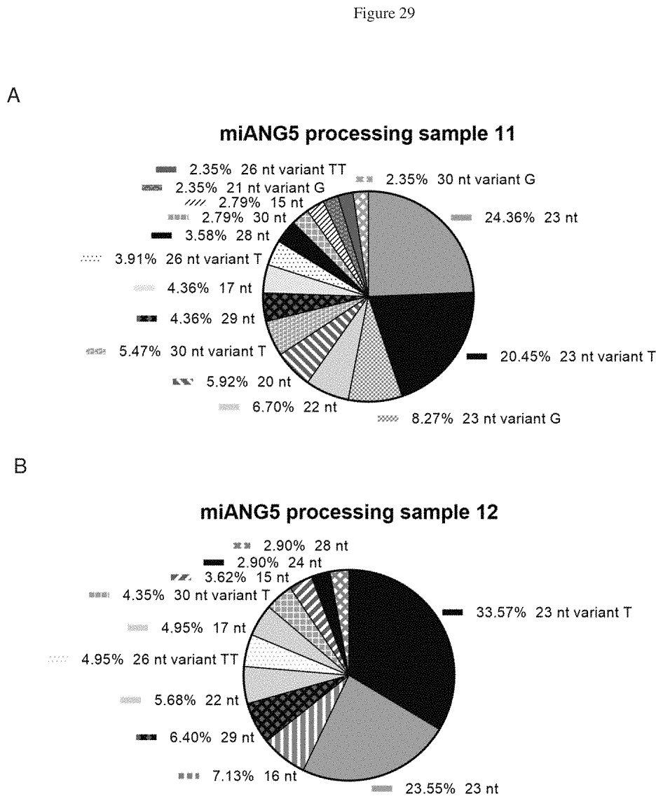

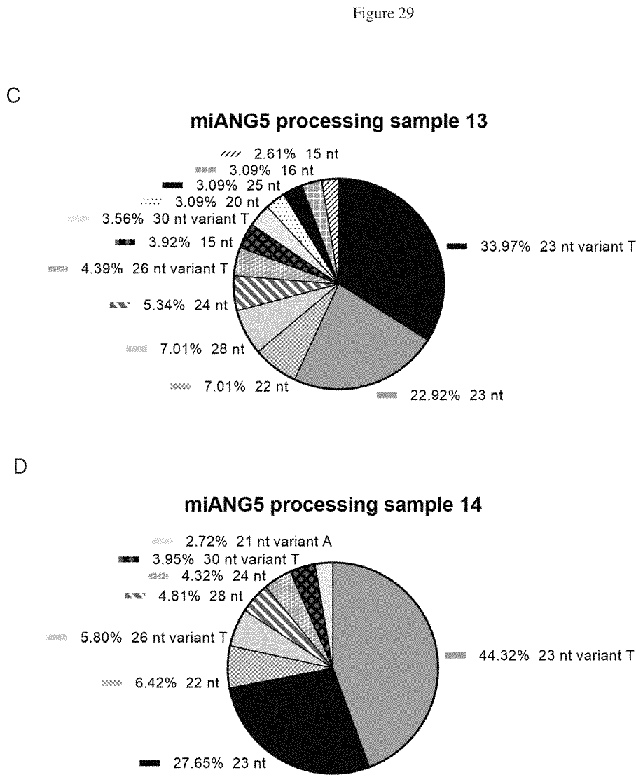

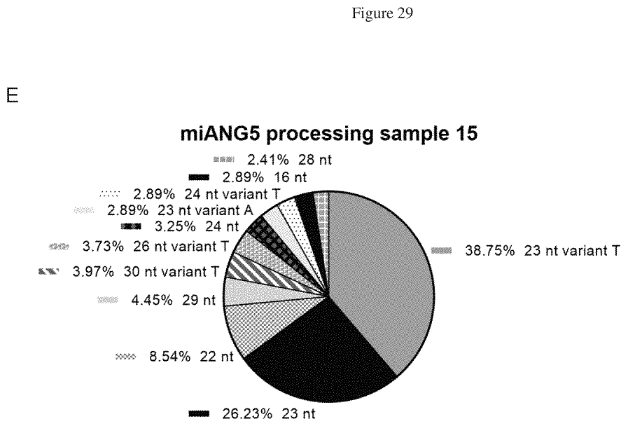

In liver samples of mice injected with AAV5-miANG5 (experimental samples 11-15, injection dose 5e13 gc/kg) the length of the mature miANG5 forms was investigated. The miRNAs processing was analyzed by alignment of the reads to the pre-miRNA sequences SED ID. NOs.66. The top 20 most abundant mature forms obtained from the annotation process were considered for graphical purposes and set to 100%. Two mismatches with the reference sequences were allowed and the reads represented with less than 2% are not shown. In samples 12-15 observed mismatches with the reference sequence were observed in sequence modification at the 3′ in which adenine or thymine (uracil) was added to the mature guide sequences (FIGS. 29.B, 29.C, 29.D and 29.E). Sample 11 showed greater sequence modification at the 3′ in which adenine, thymine (uracil) or guanine was added to the mature guide sequences (FIG. 29.A). The length of the most abundant form for mature miANG5 is 23 nts in samples 11 (5′-TAGCAAATCTTGATTTTGGCTCC-3′) or 23 nts with one nucleotide variant in samples 12-15 (5′-TAGCAAATCTTGATTTTGGCTCT-3′).

### Silencing Efficacy of AAV5-miANG5 Vector in Wild Type Mice

To investigate the safety and silencing efficacy of miANG5 in vivo, AAV5 vectors were generated encoding miANG5 and miANG-SCR1 that served as negative control. C57BL/6 female mice were IV injected in their tail vein using a low dose of 1E+13 gc/kg, a mid dose of 5E+13 and a high dose of 2.5E+14 gc/kg (n=6). The miANG-SCR1 control group only received the highest dose of 2.5E+14 gc/kg.

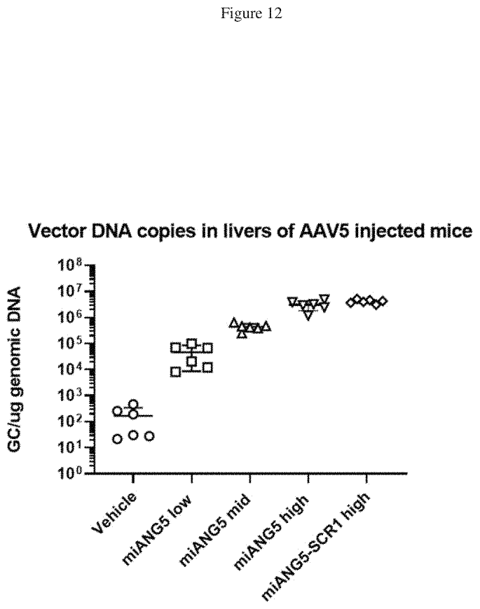

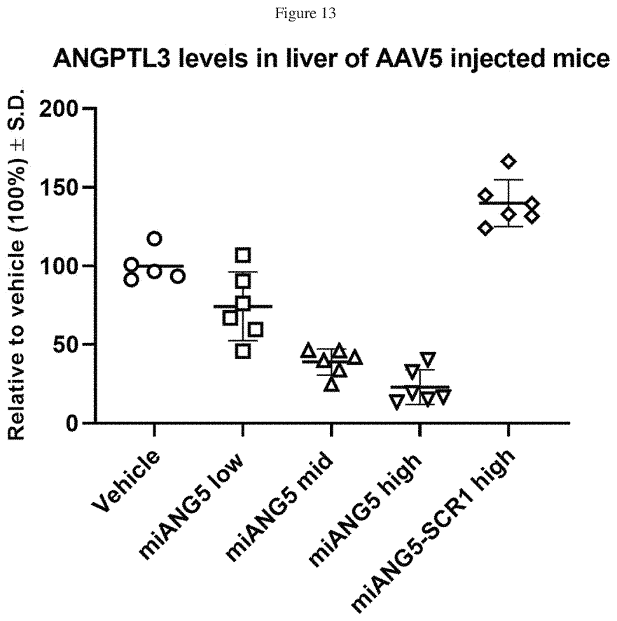

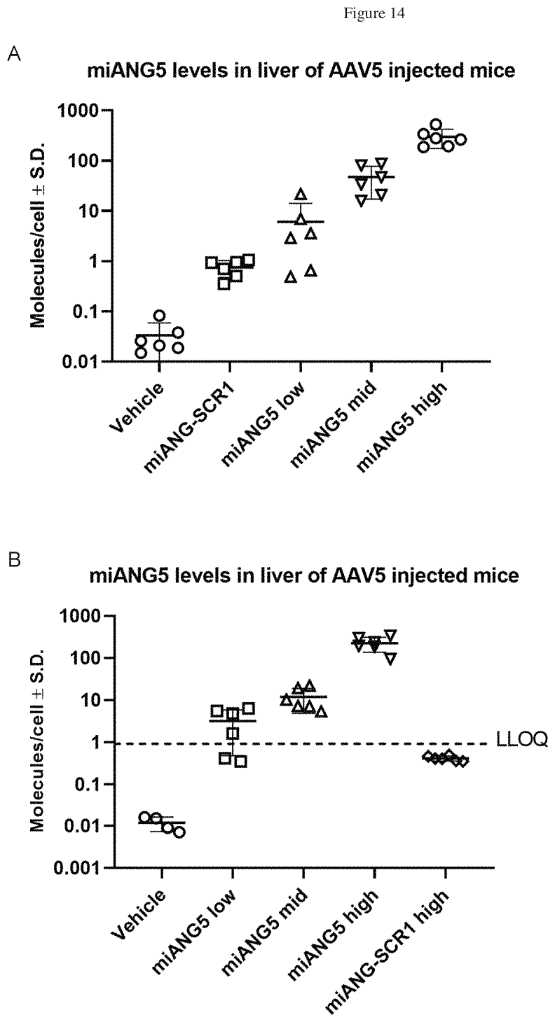

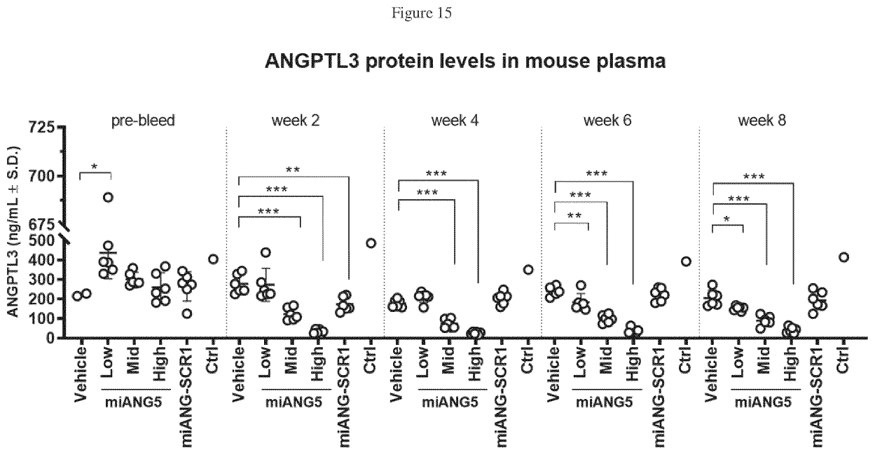

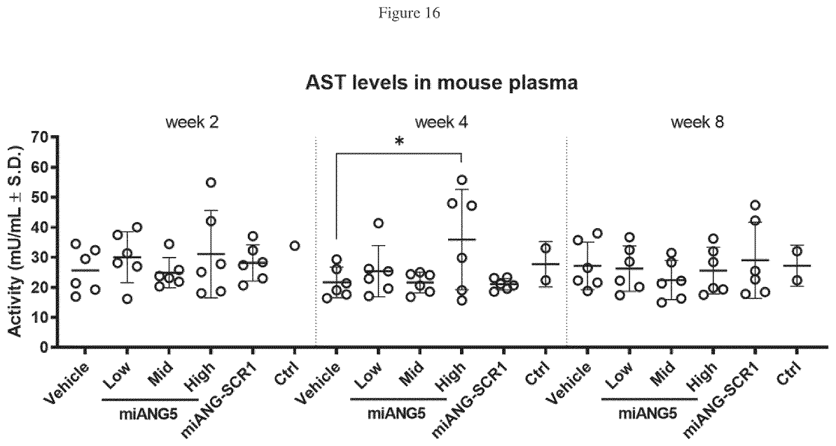

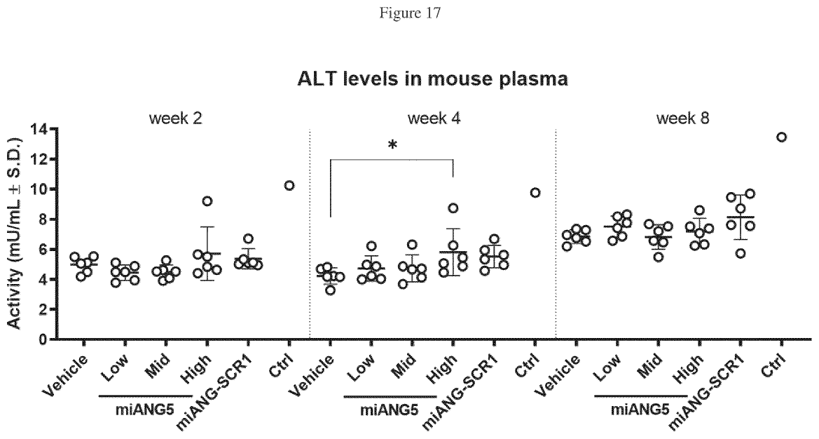

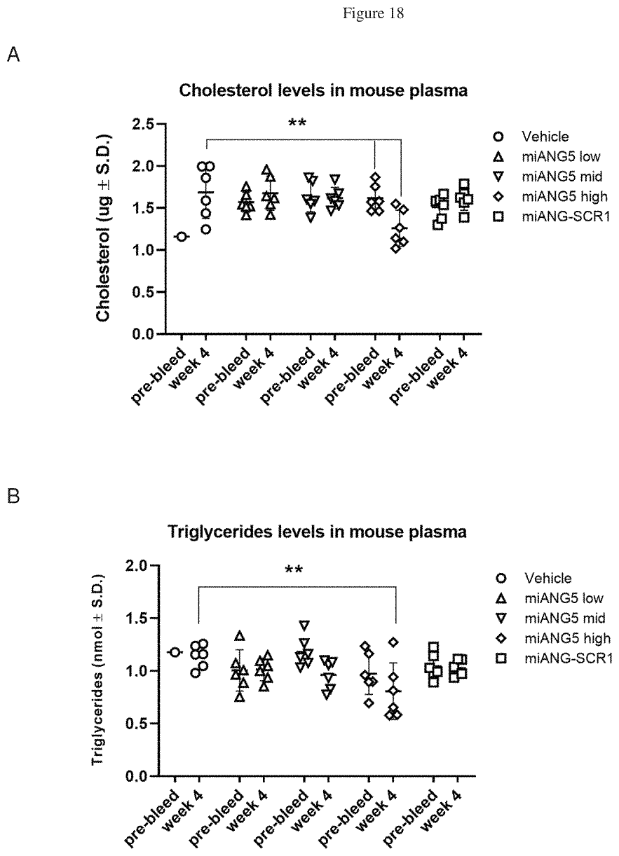

Vector DNA measurements showed that there were no mis-injected animals and that the increase on the gc number was dose-dependent, with an average of 4.7E+4 gc/μg of DNA in miANG5 low dose, 4.4E+5 gc/μg of DNA in miANG5 mid dose and 3.1E+6 and 4.2E+6 gc/μg of DNA respectively in miANG5 and miANG5-SCR high dose (FIG. 12). There is a clear dose-dependent pattern in the decrease in mouse Angptl3 mRNA expression upon AAV5-miANG5 injection. A maximum decrease of approximately 77% was reached at the high dose compared to the vehicle injected group, followed by 60% and 25% at mid and low dose (FIG. 13). The group injected with AAV5-miANG-SCR1 showed approximately 40% of increased Angptl3 mRNA expression compared to the vehicle group, which was not reflected in a higher ANGPTL3 plasma protein level suggesting that this is due to assay variability. In a follow-up study, APOE*3-Leiden.CETP mice were injected with miANG-SCR1 at a lower dose (5E+13 gc/kg vs. 2.5E+14 gc/kg) and no difference in the Angptl3 expression level compared to the vehicle group was observed. In line with the vector DNA and mRNA results, a dose-dependent increase of mature miANG5 (of 24 nts or 23 nts variant T) was detected in the livers; an average of ˜6±8 molecules/cell was calculated for miANG5 low dose, ˜47±30 molecules/cell in miANG5 mid dose and ˜299±125 molecules/cell in miANG5 high dose (miANG5 24 nts, FIG. 14.A). An average of ˜3±3 molecules/cell was calculated for miANG5 low dose, ˜12±7 molecules/cell in miANG5 mid dose and ˜224±86 molecules/cell in miANG5 high dose (miANG5 23 nts variant T, FIG. 14.B). The 23 nts variant T is the most abundant mature form of miANG5 in mouse livers, the higher number of miANG5 24 nts molecules/cell detected is most probably due to the assay background. Expression of miANG5 induced a strong and dose-dependent lowering of the circulating ANGPTL3 protein (FIG. 15) in mice. Up to 90% of ANGPTL3 protein knockdown was detected in the highest dose group and ˜50% and 25% knockdown in ANGPTL3 protein levels in the mid and low dose group. ALT and AST were measured to monitor liver function. The concentration of ALT in hepatic cell cytoplasm is comparable to AST and in all other tissues, in particularly that ALT activity is significantly less than AST (Vroon, D H., and Israili, Z., 1990). AST levels at week 2 and 8 post-AAV injection were not elevated in the miRNA expressing mice (miANG or miANG-SCR1) compared to vehicle-injected mice. A small significant increase was observed for AST at week 4 post-AAV treatment compared to the vehicle group (FIG. 16). The wider spread of AST activity levels within animals of the same group, compared to AST levels, is probably due to a higher physiological variation. Hepatic cell injury usually results in 10 to 20 times increased AST levels; therefore the observed small elevation was not related to a pathological condition (Vroon, D H., and Israili, Z., 1990). Measurements of ALT at week 2 and 8 post-AAV injection in mice did not show a significant increase compared to vehicle-injected mice. A transient increase was found in the miANG5 high dose injected mice at only week 4 (FIG. 17). Hepatocellular injury in mice following the AAV administration is characterized by a much higher and sustained ALT level (Borel, F. et. al., 2011). This suggest that it is likely related to assay or physiological mice variations instead of a pathological ALT elevation. At week 4 post-injection, TC and TG levels were significantly lower in the highest dose group receiving AAV5-miANG5 compared to the vehicle group, and solely for TC level, also when compared to pre-bleed measurement (FIGS. 18.A and B).

### Silencing Efficacy of AAV5-miANG5 and AAV5-miANG10 Vectors in APOE*3-Leiden.CETP Transgenic Mice

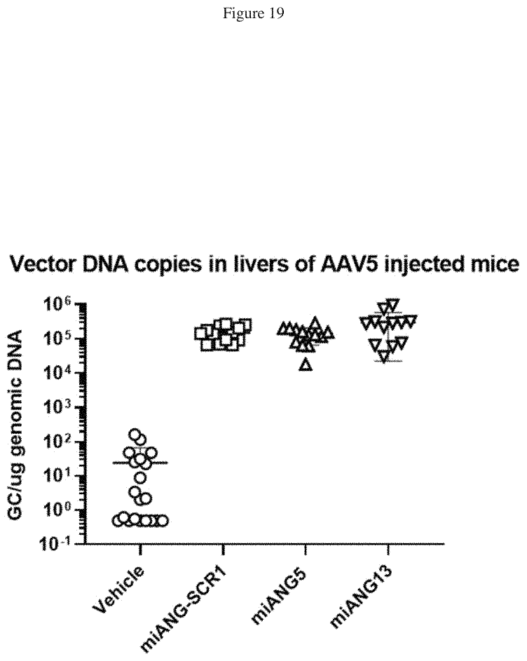

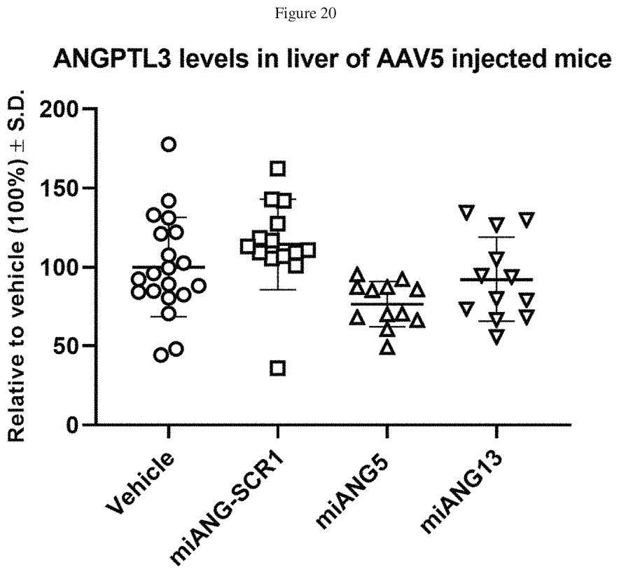

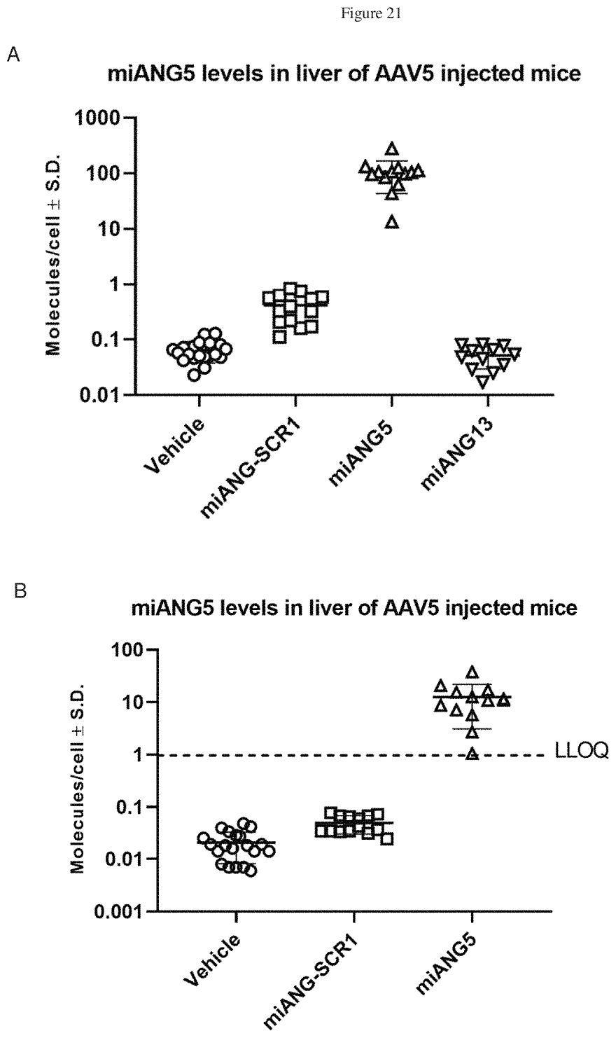

The APOE*3-Leiden.CETP mouse was used to study miANG5 and miANG13 efficacy. This mouse model possesses human characteristics with respect to lipid metabolism, including a reduced HDL/LDL ratio and increased susceptibility to diet-induced atherosclerosis and responds similarly as humans to all registered hypolipidemic drugs, such as statins, fibrates, niacin, ezetimibe and anti-PCSK9 monoclonal antibodies. Hyperlipidemia was induced by using a Western and cholesterol-containing diet. APOE*3-Leiden.CETP female mice were IV injected using a dose of 5E+13 of AAV5-miANG5, AAV5-miANG13 or AAV5-miANG-SCR1 (n=15). At week 16, the animals were sacrificed, and subsequently the vector genome copies, Angptl3 mRNA expression, ANGPTL3 protein and miANG5 (23 nts variant T mature form) levels in the livers were determined. Body weight, food intake, TC, TG, lipids profile, plasma ANGPTL3 protein expression and ALT/AST levels were assessed up to 16 weeks for AAV5-miANG5 and AAV5-miANG5-SCR1 and up to 12 weeks for AAV5-miANG13. Equal vector gc numbers were detected within the groups with an average of 1.38E+05 gc/μg of DNA in AAV5-miANG5, 3.01E+05gc/μg of DNA in AAV5-miANG13 and 1.41E+05 gc/μg of DNA in AAV5-miANG5-SCR1, respectively (FIG. 19). Few animals were partially dosed and were excluded from all analyses: mouse 36 and 45 from AAV5-miANG5 group; mouse 51, 54 and 61 from AAV5-miANG13 group and mouse 24 in AAV5-miANG-SCR1 group having <3.07E+03 gc/μg of DNA. Approximately 30% decrease in the mouse Angptl3 mRNA expression was observed in the group injected with AAV5-miANG5, while no effect was observed in AAV5-miANG13 and AAV5-miANG-SCR1 injected mice (FIG. 20). The mild decrease of Angptl3 mRNA expression at week 16 is consistent with the ANGPTL3 protein expression data at week 12 and 16. The miANG5 24 nts mature form was detected and the expression was on average 105±62 molecules/cell (FIG. 21.A. The mature form of miANG5 of 23 nts variant T was also detected and the expression was on average 13±9 molecules/cell (FIG. 21.B). The 23 nts variant T is the most abundant mature form of miANG5 in mouse livers, the higher number of miANG5 24 nts molecules/cell detected is most probably due to the assay background.

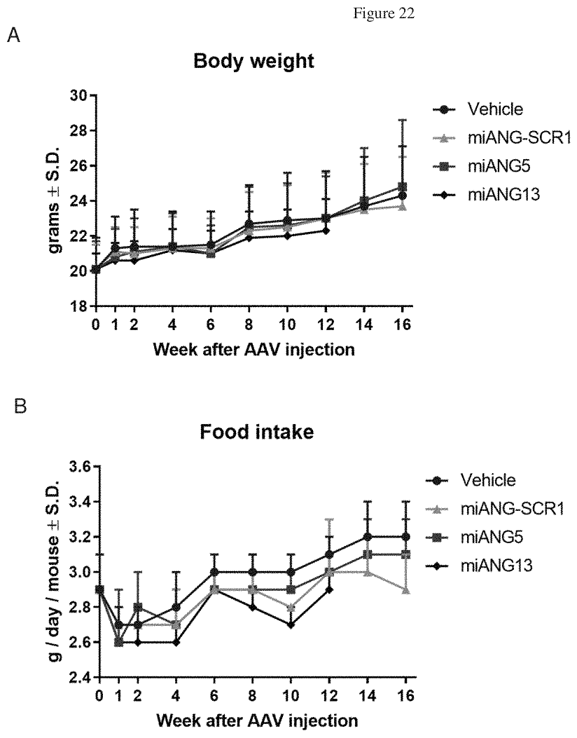

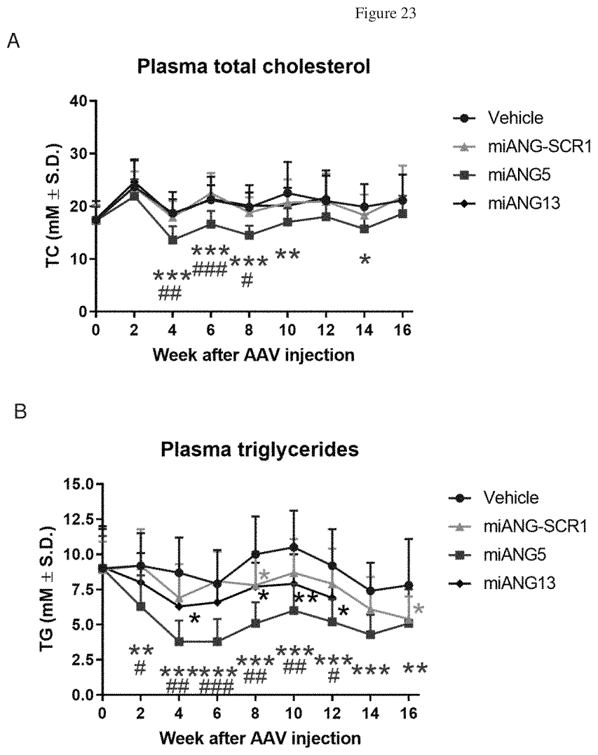

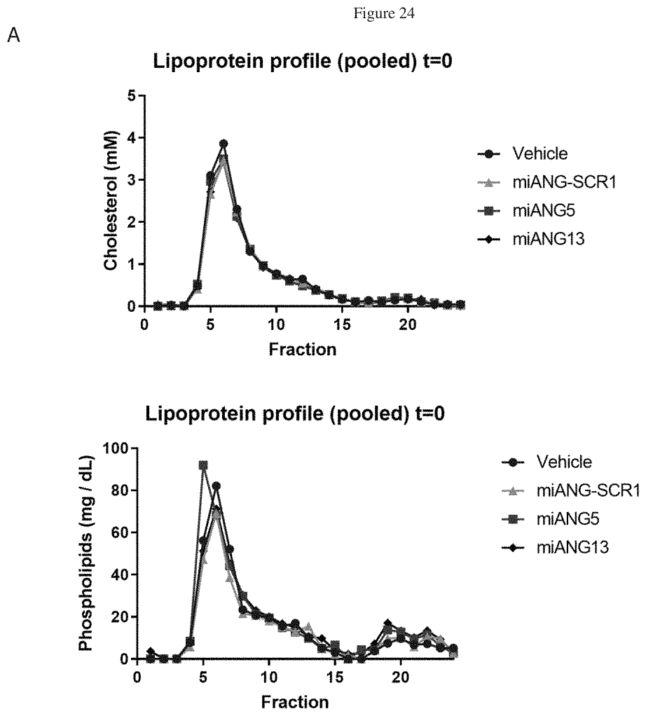

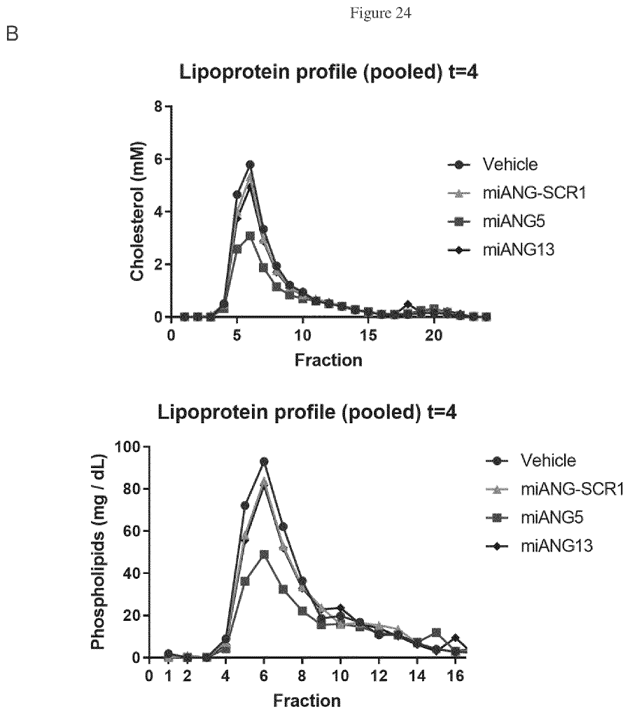

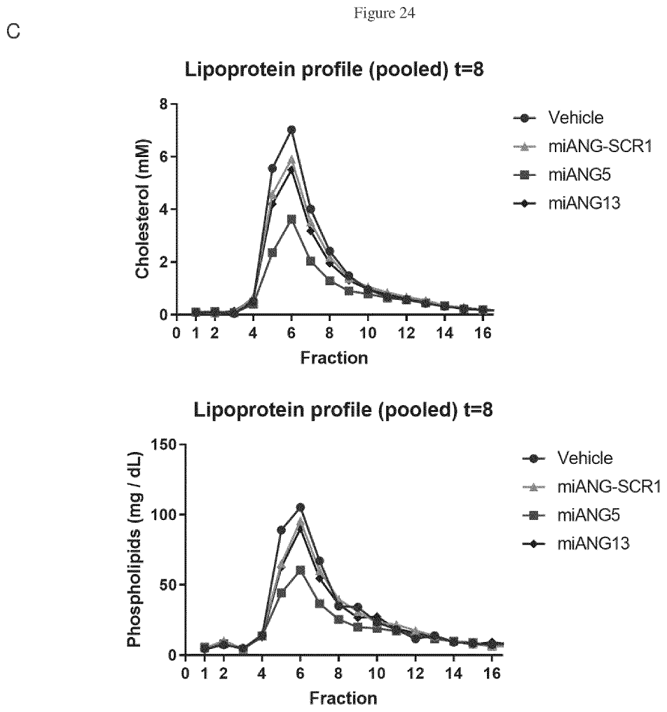

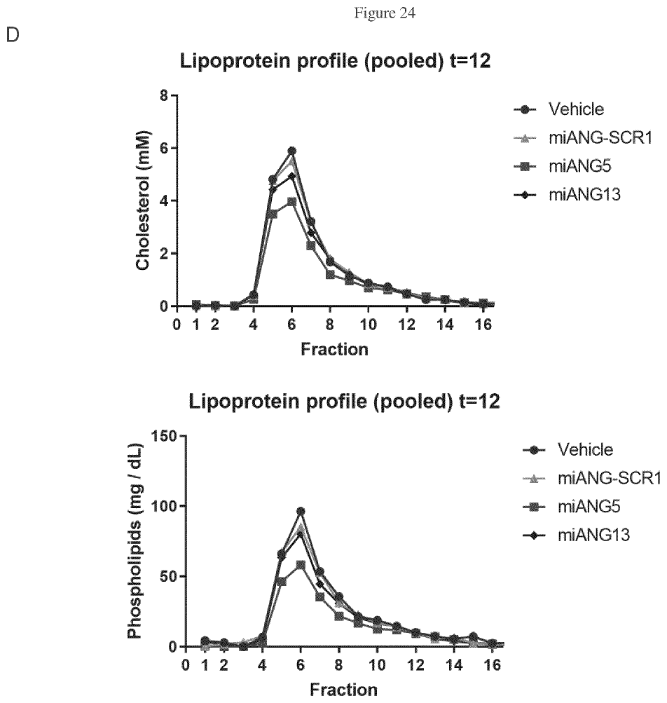

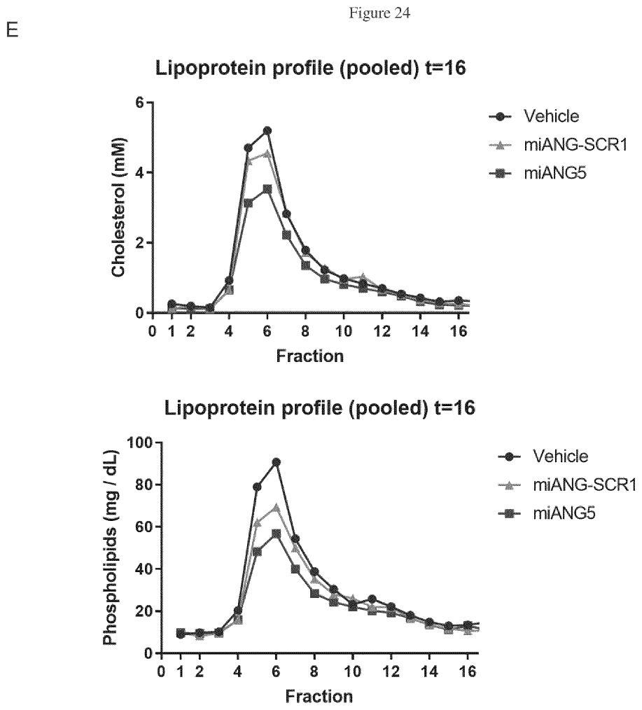

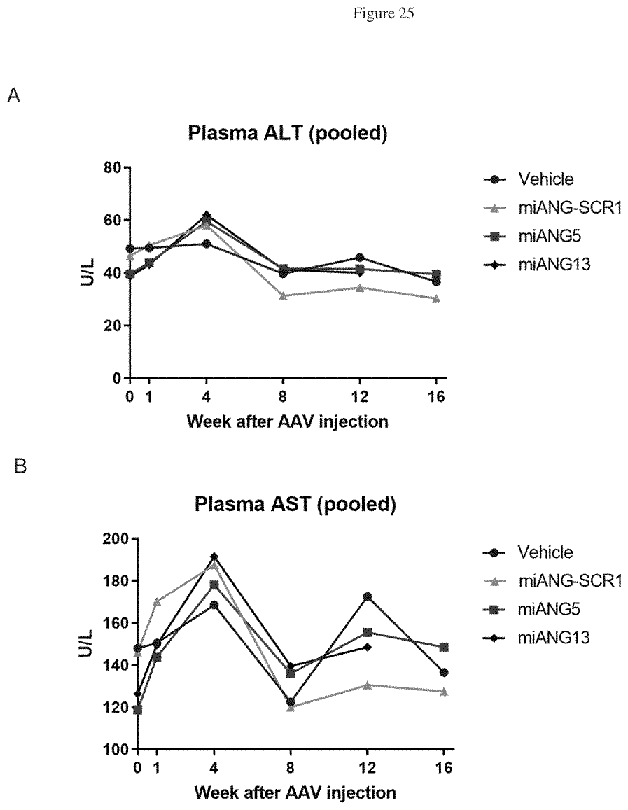

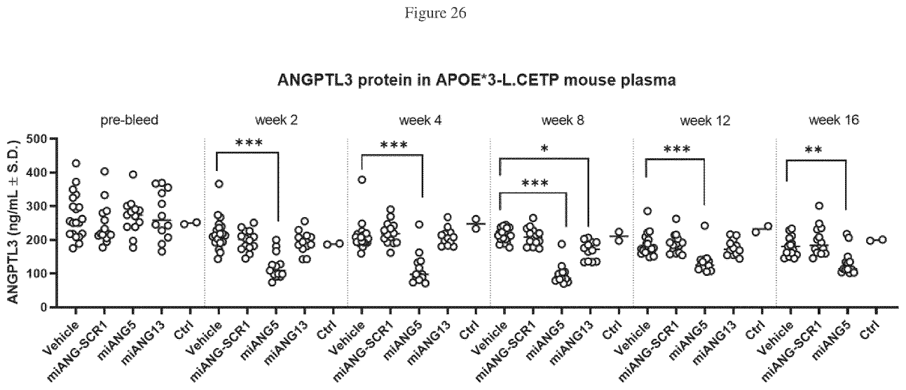

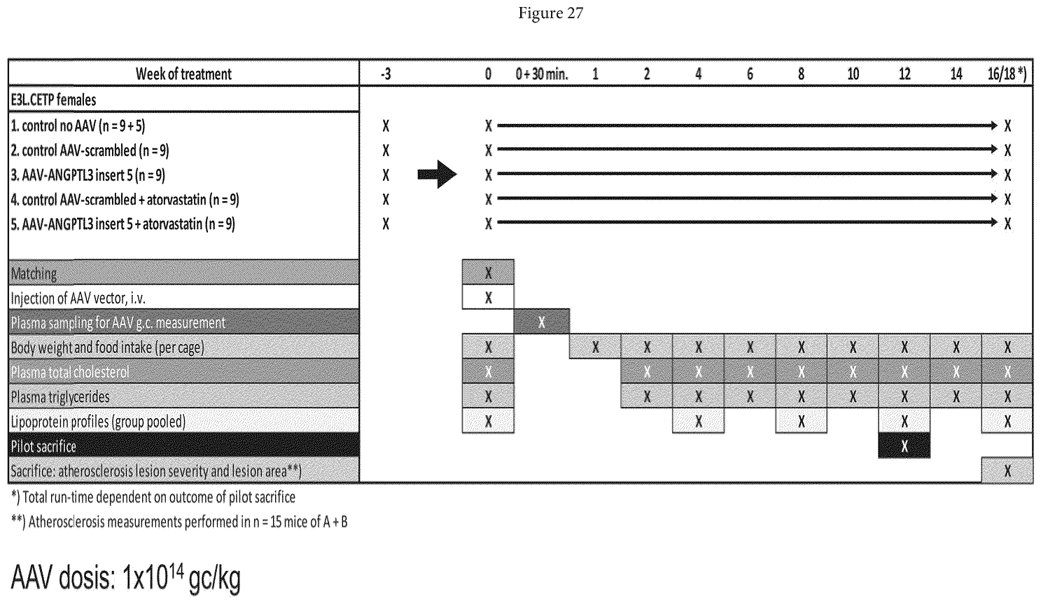

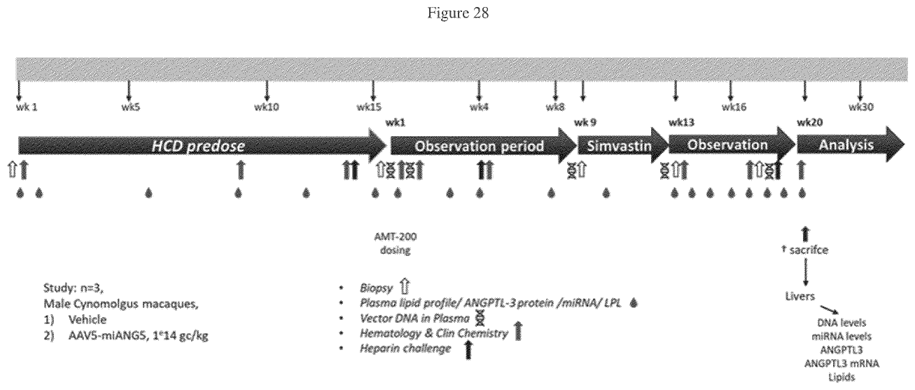

The results showed no differences in body weight and food intake between the vehicle group and AAV5 injected groups throughout the duration of the study (FIGS. 22.A and B). Plasma total cholesterol was lowered by AAV5-miANG5 administration at week 4 (˜25%), 6 (˜29%) and 8 (˜22%), and was lowered by AAV5-miANG-SCR1 at week 4 (˜27%), 6 (˜24%), 8 (˜25%), 10 (˜24%) and 14 (˜20%), when compared to the vehicle group (FIG. 23.A). Plasma TG were also significantly lower (up to ˜58%) at week 4 in the AAV5-miANG5 group versus the SCR and vehicle group (FIG. 23.B). The group receiving AAV5-miANG13 did not show a consistent effect. The plasma TG levels of the AAV5-miANG13 group at week 4 (˜27%), 8 (˜23%), 10 (˜26%) and 12 (˜27%) were significantly reduced compared to those of the vehicle group, but not when compared to the AAV5-miANG-SCR1 group. This is due to the reduction of TG level in the AAV5-miANG5-SCR1 control group at week 8 (˜20%) and 16 (˜29%) compared to the vehicle group. Lipoprotein profiles data showed that the decrease in plasma total cholesterol and TG observed in AAV5-miANG5 group occurred in the (V)LDL fraction. A reduction in the (V)LDL fraction was observed from week 4 up to week 16 (FIGS. 24.A-.E). In the pooled plasma samples, the levels of ALT/AST hepatotoxicity markers were within the normal range, indicating no liver related injury due to the AAV-treatment (FIGS. 25.A and B). ANGPTL3 protein was significantly lower in plasma of AAV5-miANG5 injected APOE*3-Leiden.CETP mice throughout the entire study, whereas AAV5-miANG13 injected mice showed only a minor (˜20%) but significant decrease in week 8 post-IV (FIG. 26. The maximum silencing effect of miANG5 was reached at week 8 with a ˜54% decrease in ANGPTL3 protein expression compared to the vehicle group and the group injected with AAV5-miANG-SCR1.

Silencing Efficacy of AAV5-miANG5 Alone or in Combination with Atorvastatin on Atherosclerosis Development in APOE*3-Leiden.CETP Transgenic Mice

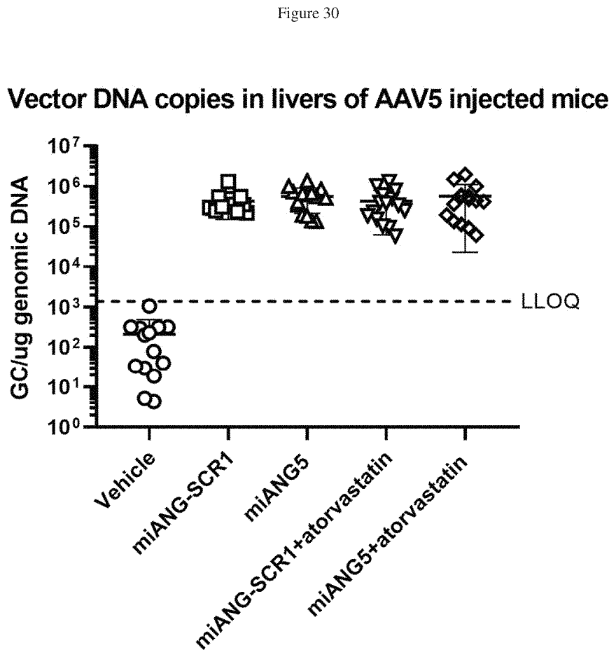

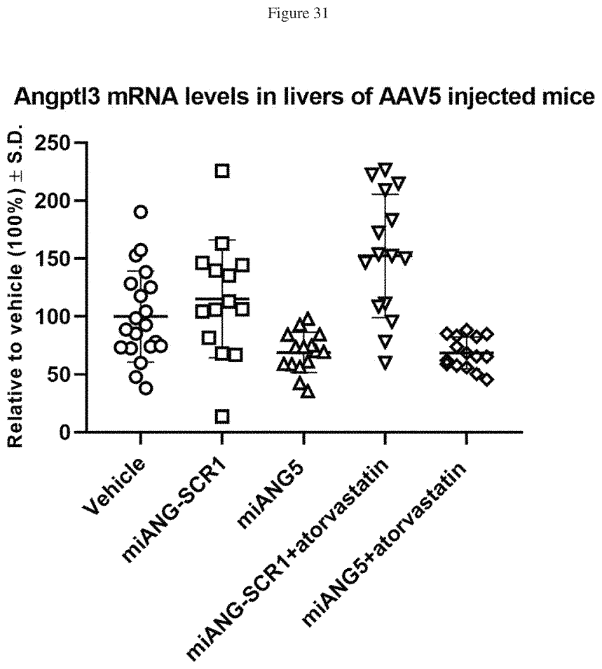

The aim of this study was to examine the effect of AAV5-miANG5 to induce gene silencing of ANGPTL3, alone or in combination with atorvastatin treatment, on development of atherosclerosis in APOE*3-Leiden.CETP mice. Hyperlipidemia was induced by using a Western and cholesterol-containing diet. APOE*3-Leiden.CETP female mice were injected intravenously with vehicle solution (n=20), 1E+14 gc/kg of AAV5-miANG5 (alone or in combination with atorvastatin, n=15 per group) or AAV5-miANG-SCR1 (alone or in combination with atorvastatin, n=15 per group). At week 16, the animals were sacrificed, and subsequently the vector genome copies, Angptl3 mRNA expression, ANGPTL3 protein and miANG5 (23 nts variant T mature form) levels in the livers were determined. Body weight, food intake, TC, TG, lipids profile, plasma ANGPTL3 protein expression and ALT/AST levels were assessed up to 16 weeks. Atherosclerosis measurements (severity and lesion area) in aortic root in week 12 (in 5 pilot mice) and week 16 (in 15 mice per group) were performed. Equal vector gc numbers were detected within the groups with an average of 4.26E+05 gc/μg of DNA in AAV5-miANG-SCR1, 5.56E+05gc/μg of DNA in AAV5-miANG5, 4.27E+05gc/μg of DNA in AAV5-miANG-SCR1+atorvastatin and 5.65E+05 gc/μg of DNA in AAV5-miANG5+atorvastatin, respectively (FIG. 30). Vector DNA measurements showed that mouse 31 (belonging to the AAV5-miANG-SCR1 group) was most likely misinjected animals with gc/μg of DNA below the LLOQ. Approximately 30% decrease in the mouse Angptl3 mRNA expression was observed in the group injected with AAV5-miANG5 and AAV5-miANG5 with atorvastatin (FIG. 31). Similarly, as observed in the previous study in APOE*3-Leiden.CETP mice, the Angplt3 mRNA expression in the vehicle and the AAV5-miANG-SCR groups showed highly variable levels. Quantifiable levels of miANG5 23 nts variant T were detected only in the AAV5-miANG5 injected groups with an average of ˜87 and ˜107 molecules/cell respectively in the group treated with AAV5-miANG5 alone and in combination with atorvastatin (FIG. 32).

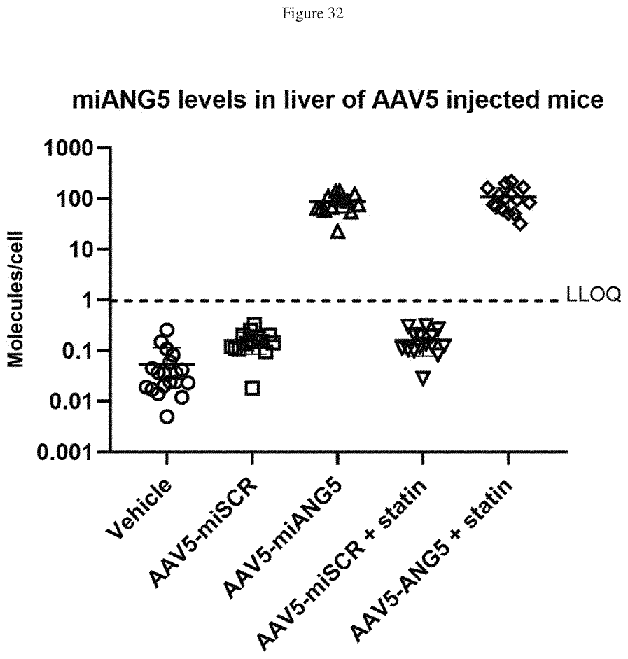

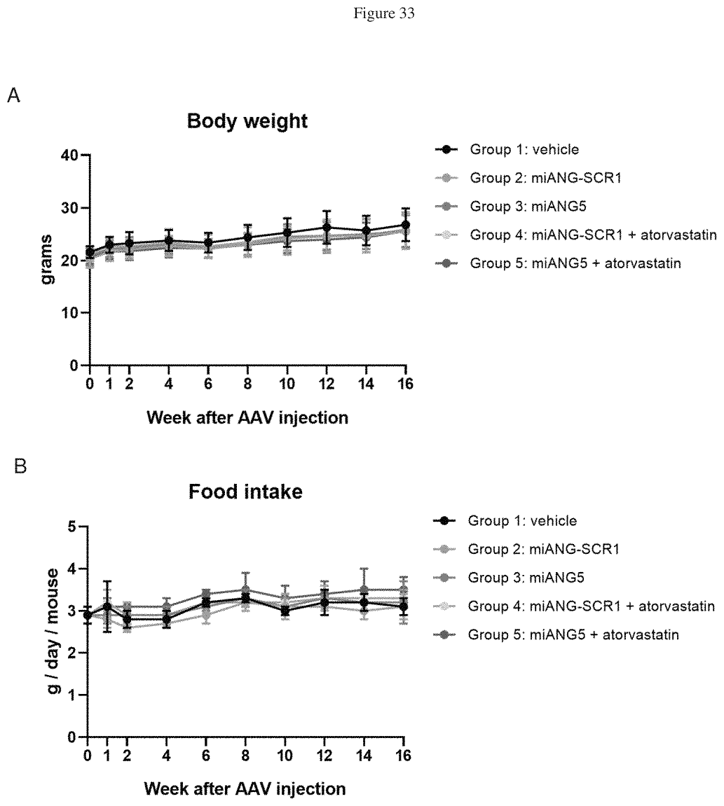

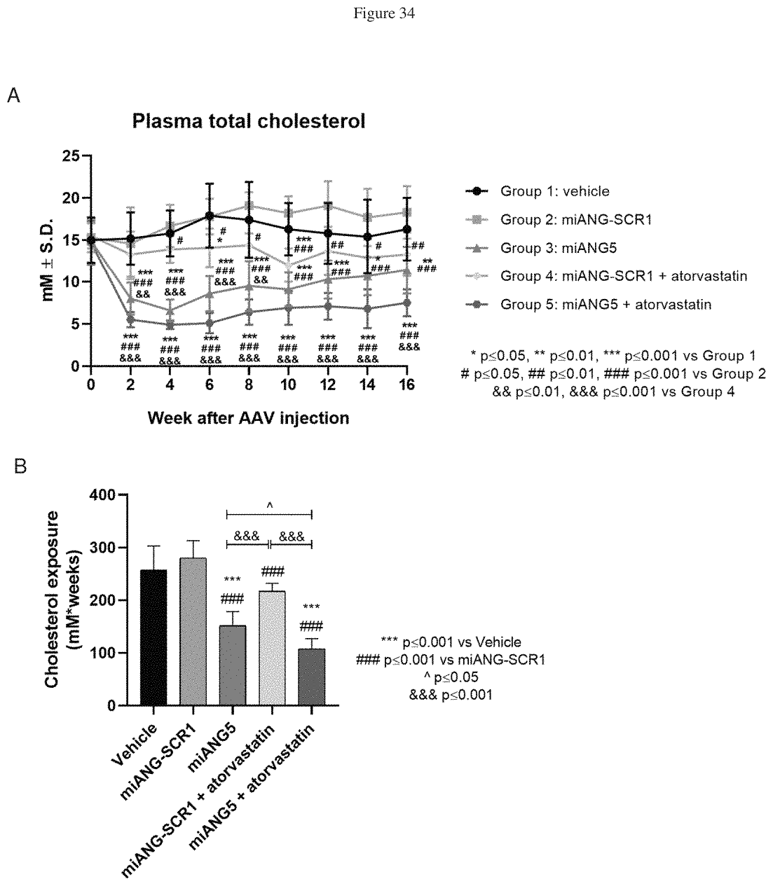

The results showed no differences in body weight and food intake between the vehicle group and AAV5 injected groups throughout the duration of the study (FIGS. 33.A and B). Plasma total cholesterol levels measured in the vehicle group were approximately 15-18 mmol/L during the study (group 1) and for the miANG-SCR1 group approximately 15-19 mmol/L (group 2). Thus, there were no differences in plasma total cholesterol levels between the vehicle and the scrambled control group at any of the time points (FIG. 34.A). Animals treated with AAV5-miANG5 only (group 3) had decreased plasma total cholesterol levels compared to both the vehicle control group and the scrambled control group in all weeks after start of treatment. The average reduction per sample point in plasma total cholesterol after injection of AAV5 was −43% compared to the vehicle control group and −48% compared to the scrambled miRNA control group. Based on the cholesterol exposure, the average decrease in plasma cholesterol up to study week 16 was −41% and −46% compared to the vehicle control group and the scrambled control group, respectively (FIG. 34.A). Animals treated with scrambled miANG-SCR1 and atorvastatin (group 4) showed a decrease in plasma total cholesterol levels compared to the vehicle control group in weeks 6 (˜21%) and 10 (˜27%) of the study. Compared to the scrambled control, plasma total cholesterol levels were decreased from week 4 up to week 16 of the study. From week 8 up to week 16 of the study, the decrease in plasma total cholesterol was ≥−25% (FIG. 34.A). Animals treated with AAV5-mANG5 in combination with atorvastatin (group 5) had decreased plasma total cholesterol levels compared to both the vehicle control group and the scrambled control group in all weeks after start of treatment. The average reduction per sample point in plasma total cholesterol after injection of AAV5 was −61% compared to the vehicle control group, −64% compared to the scrambled miRNA control group, −53% compared to the atorvastatin-treated group, and −32% compared to the group treated with AAV5-miANG5 only (FIG. 34.A). Based on the cholesterol exposure, the average decrease in plasma cholesterol up to study week 16 was −58% and −61% compared to the vehicle control group and the scrambled control group, respectively. Compared to treatment with miANG-SCR1, treatment with AAV5-miANG5 and atorvastatin decreased plasma total cholesterol levels in all weeks after start of treatment. Based on the cholesterol exposure, the average decrease in plasma cholesterol up to week 16 was −50%. Compared to treatment with AAV5-miANG5 only, combination treatment did not statistically significantly affect plasma total cholesterol levels at any of the individual time points, however trends towards a significant difference were found in weeks 6, 14 and 16, and cholesterol exposure showed statistically significant decrease (−29%) (FIG. 34.B).

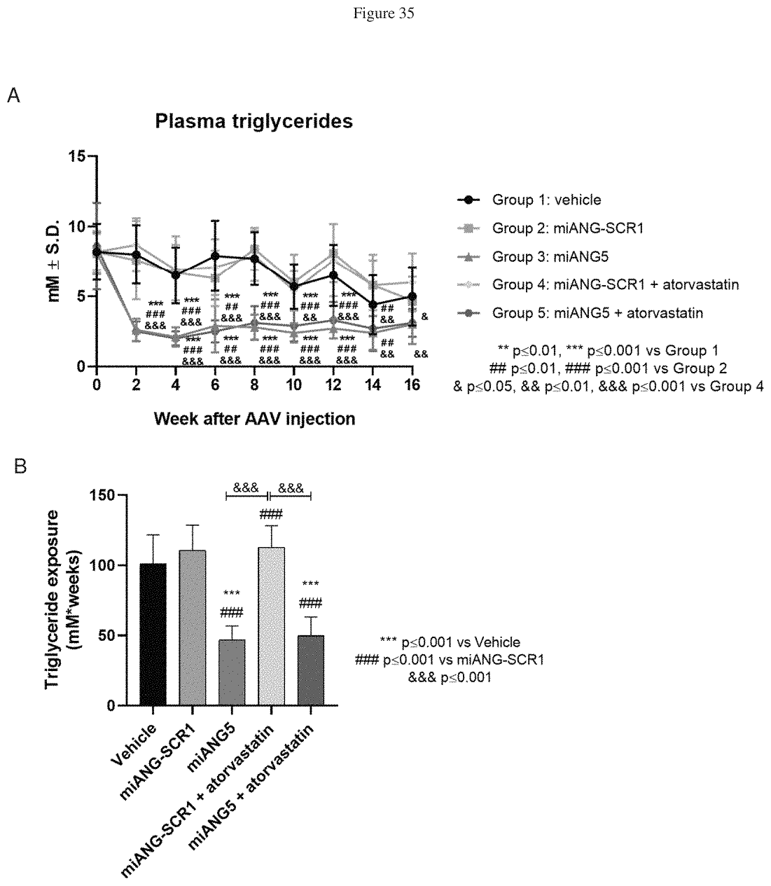

The vehicle control group (group 1) showed plasma triglyceride levels of approximately 4-8 mmol/L during the study and the miANG-SCR1 group (group 2) levels of approximately 5-8 mmol/L. Thus, there were no differences in plasma triglyceride levels between the vehicle and the scrambled control group at any of the time points (FIG. 35.A). Animals treated with AAV5-miANG5 only (group 3) had decreased plasma triglyceride levels compared to the vehicle control group and the scrambled control group from week 2 up to week 12 and 14 of the study, respectively. The average reduction per sample point in plasma triglycerides after injection of AAV5 was −60% compared to the vehicle control group and −61% compared to the scrambled miRNA control group. Based on the triglyceride exposure, the average decrease in plasma triglycerides up to week 16 was −54% and −58% compared to the vehicle control group and the scrambled control group, respectively (FIG. 35.A). Animals treated with scrambled miRNA and atorvastatin (group 4) showed no difference in plasma triglyceride levels compared to both controls. Animals treated with AAV5-miANG5 in combination with atorvastatin (group 5) had decreased plasma triglyceride levels compared to the vehicle and the scrambled control groups from week 2 up to week 12 and 14 of the study, respectively. The average reduction per sample point in plasma total triglycerides after injection of AAV5 was −57% compared to the vehicle control group, −59% compared to the scrambled miRNA control group, and −60% compared to the atorvastatin-treated group (FIG. 35.A). Based on the triglyceride exposure, the average decrease in plasma triglycerides up to week 16 was −51% and −55% compared to the vehicle control group and the miANG-SCR1, respectively. Compared to treatment with scrambled miRNA and atorvastatin, treatment with AAV5-miANG5 and atorvastatin decreased plasma triglyceride levels in all weeks after start of treatment. Based on the cholesterol exposure, the average decrease in plasma triglycerides up to week 16 was −56%. Compared to treatment with AAV5-miANG5 only, combination treatment did not affect plasma triglyceride levels at any of the study time points (FIG. 35.B).

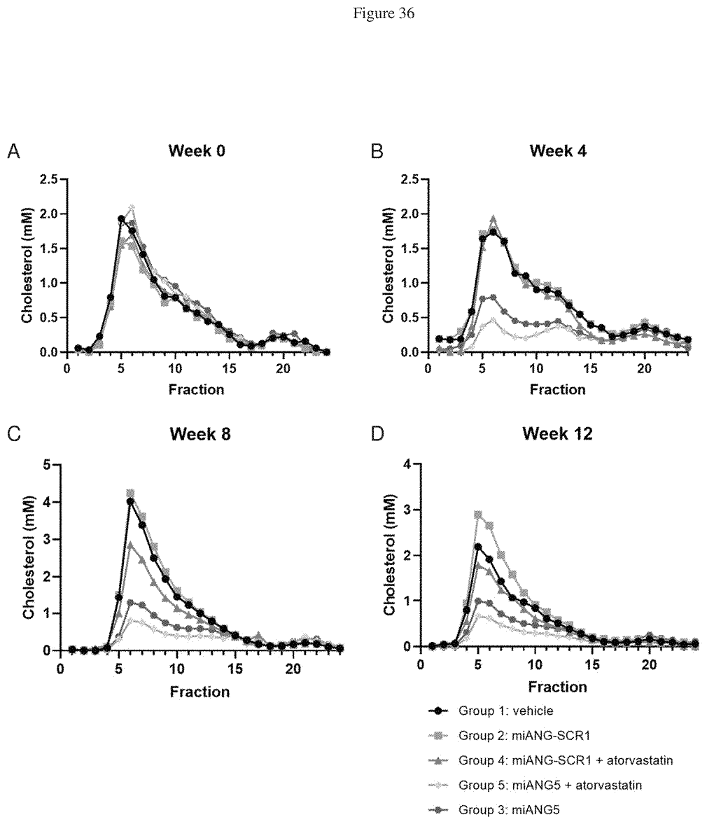

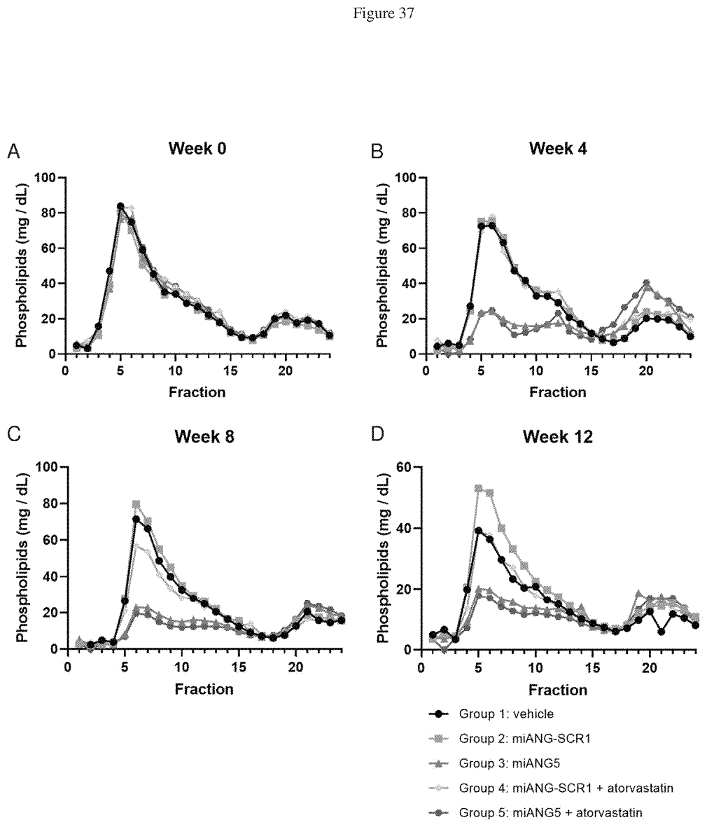

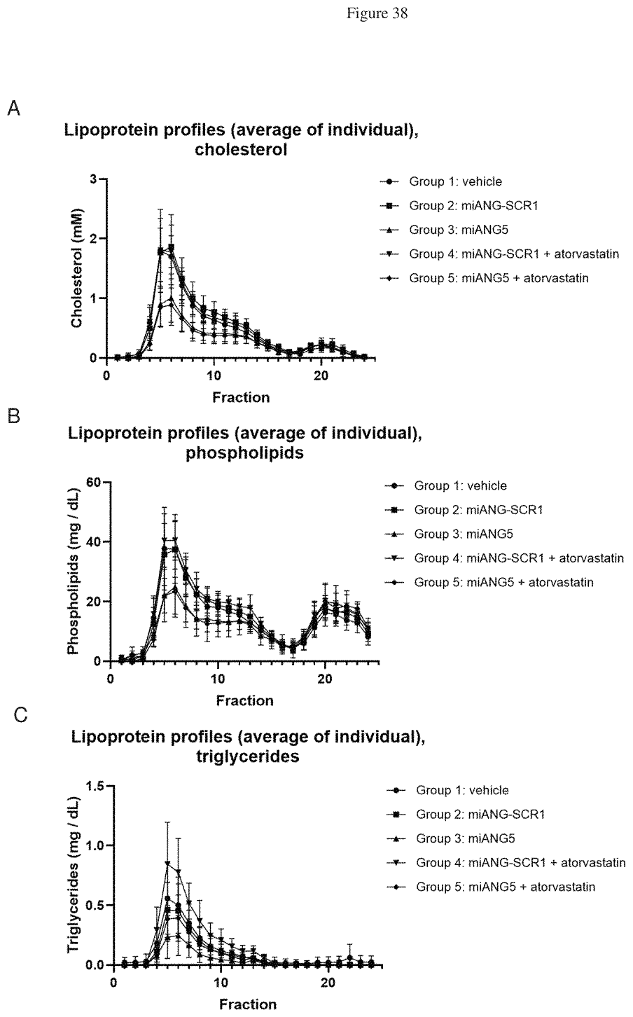

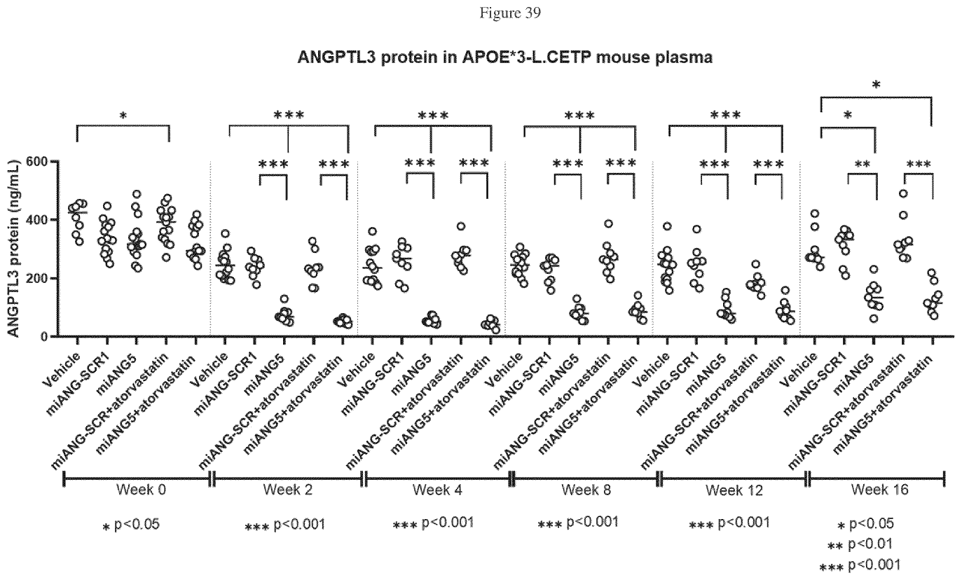

Lipoprotein measurements (cholesterol and phospholipids) were performed on pooled plasma samples per group in week 0, 4, 8, and 12 of the study (FIGS. 36 and 37), and on individual samples in week 16 of the study (FIG. 38). We consider fractions 3-8 as VLDL, 9-16 as IDL/LDL, and 17-24 as HDL. No statistics were performed for this analysis. The lipoprotein profiles confirm that injection of AAV5-miANG5 only (group 3) and AAV5-miANG5 in combination with atorvastatin (group 5) has cholesterol-lowering effect and reduces VLDL-cholesterol and LDL-cholesterol but does not appear to have an effect on HDL-cholesterol. ANGPTL3 plasma protein was significantly lowered in APOE*3-Leiden.CETP mice injected with AAV5-miANG5 only throughout the entire 16-weeks study when compared to the vehicle group and miRNA scramble group (˜78% lowering at week 4 compared to miANG-SCR1; FIG. 39). Animals treated with AAV5-miANG5 and atorvastatin showed significant ANGPTL3 plasma protein lowering during the whole study (˜83% lowering at week 4 compared to miANG-SCR1) and no additive effect on the plasma protein lowering was observed when compared with mice injected with miANG5 only. In miANG-SCR1 group no significant differences in ANGPTL3 plasma protein levels were observed when compared to the vehicle control group. In the group treated with miANG-SCR1 and atorvastatin a significant decrease in ANGPTL3 plasma protein level was observed only at week 12 (FIG. 39).

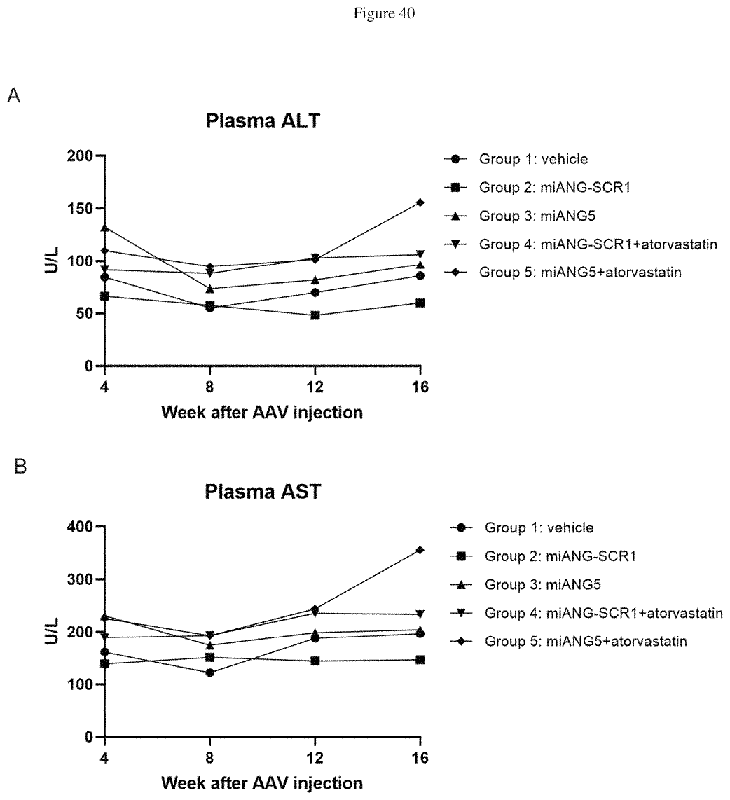

In mouse livers, ANGPTL3 protein level was significantly decreased in the AAV5-miANG-SCR1 and atorvastatin group when compared to the vehicle (˜21% lowering) and the miANG-SCR1 control groups (˜17% lowering). The ANGPTL3 protein level was also significantly lowered in livers of mice injected with AAV5-miANG5 and atorvastatin when compared to the vehicle (˜27% lowering) and the miANG-SCR1 control groups (˜24% lowering). No significant ANGPTL3 protein lowering was observed in the group treated with AAV5-miANG5 only (FIG. 40).

In 30 APOE*3-Leiden.CETP mice (6 mice/group) on 4 different time points (study week 4, 8, 12 and 16) ALT and AST parameters were analyzed (FIGS. 40.A and B). The measured levels were considered not biologically relevant, as these changes were noted at one time point only, in absence of an apparent trend regarding duration of treatment or as no statistical significance was achieved. In addition, microscopic examination of selected tissues did not reveal any differences in microscopic alterations for any of the groups.

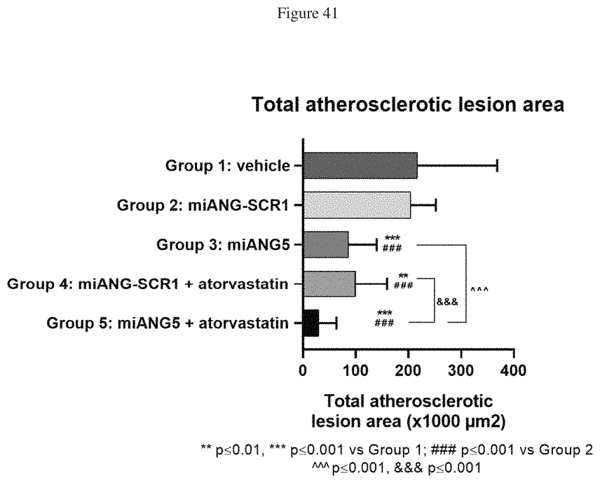

In the vehicle control group (group 1) and in miANG-SCR1 group (group 2), a mean total lesion area of 184 and 206×103 μm2, respectively, was found. In mouse 2 (belonging to group 1), a total lesion area of 685×103 μm2 was found; based on this, data for mouse 2 was excluded from further statistical analysis. Animals treated with AAV5-mANG5 only (group 3) had a reduced total lesion compared to vehicle control group (−53%) and the miANG-SCR1 group (−58%). Animals treated with miANG-SCR1 and atorvastatin (group 4) had a reduced total lesion compared to vehicle control group (−46%) and the miANG-SCR1 group (−52%), but not compared to the group treated with AAV5-miANG5 only (+15%). Animals treated with AAV5-miANG5 in combination with atorvastatin (group 5) had a reduced total lesion compared to vehicle control group (−84%) and the miANG-SCR1 group (−86%). Compared to animals treated with miANG-SCR1 and atorvastatin or AAV5-miANG5 only, a reduction in total lesion area of respectively −70% and −66% was found (FIG. 41).

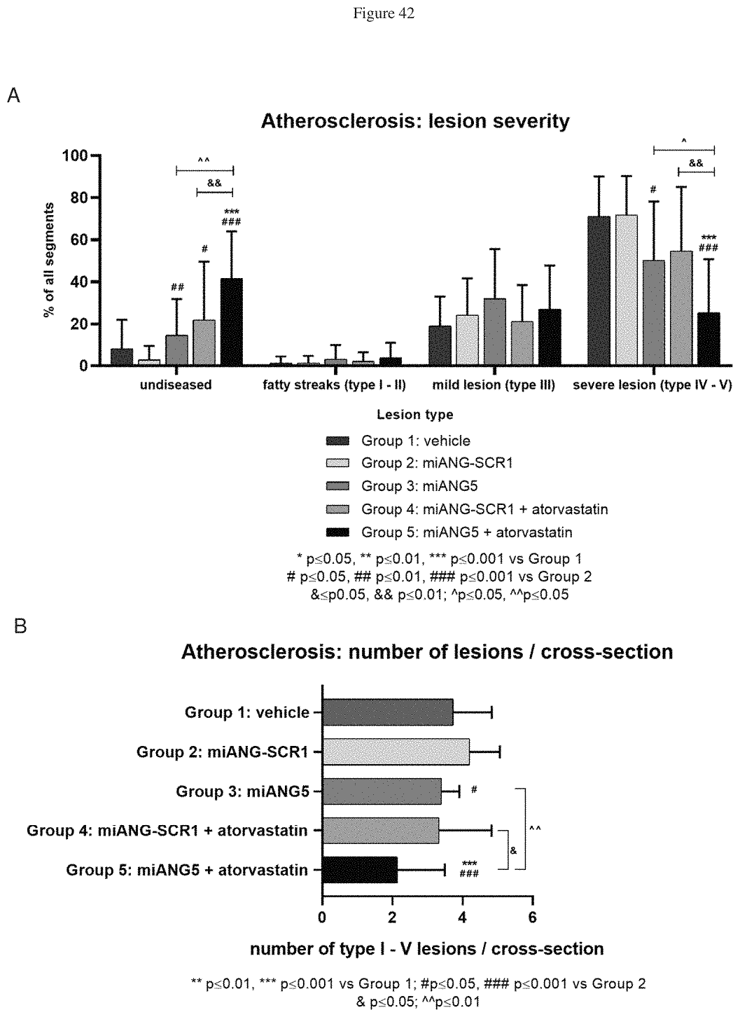

Compared to the vehicle control group (group 1), mice treated with AAV5-miANG5 in combination with atorvastatin (group 5) had a higher percentage of undiseased, normal segments in the aortic root (FIG. 42.A). Compared to the miANG-SCR1 group (group 2), mice treated with AAV5-miANG5, with atorvastatin, or their combination, had a higher percentage of undiseased, or normal segments in the aortic root. In addition, treatment with AAV5-miANG5 in combination with atorvastatin (group 5) also preserved undiseased segments when compared to treatment with AAV5-miANG5 or atorvastatin only (FIG. 42.A). Treatment with AAV5-miANG5, alone or in combination with atorvastatin, was associated with a reduction in development of severe type IV and V lesions when compared to both control groups. Treatment with AAV5-miANG5 and atorvastatin provided a further reduction in development of severe type IV and V lesions compared to single treatment with AAV5-miANG5 or atorvastatin (FIG. 42.A).

Animals treated with AAV5-miANG5 only (group 3) had a reduction in lesion number compared to the miANG-SCR1 group. Treatment with AAV5-miANG5 in combination with atorvastatin (group 5) gave a further reduction in lesion number compared to both control groups and compared to both single treatment groups (FIG. 42.B).

### Effect of a Compound for Inducing Autophagy the Transduction Efficiency of AAV Vehicles.

One million Huh7 cells per well in 1 ml of DMEM medium supplemented with 10% FBS and 1% P/S were prepared from a culture grown at 37C and 5% CO2. The cells were seeded in 6 well plates and supplemented with an additional 1 ml of fresh medium. After o/n incubation the cells are washed with 1×DPBS after which the cells where autophagy needs to be either activated or inhibited were pre-treated for 2 hours with a mixture of DMEM and Rapamycin (Activator) or Bafilomycin (Inhibitor) both at a concentration of 100 nM/ml. The cells were treated with the following conditions in 1 ml medium and where applicable AAV5 at a concentration of 5000 gc/cell and 20% intralipid at a dose of 1:64 (Sigma 1141). Treatments: a) Non-treated+AAV5, b) Activator, c) Inhibitor, d) Intralipid+AAV5, 5) Intralipid+Inhibitor+AAV5, e) Intralipid+Activator+AAV5, f) Activator+AAV5, g) Inhibitor+AAV5. All treatments were performed in biological triplicates. The cells were incubated for 4 hours before being harvested after which the DNA and RNA from the cell were isolated using the quick DNA kit from Zymo research cat no. D4074 and Promega RNA Miniprep systems cat no. Z6010 respectively. In total 25 ng of DNA was used in a qPCR reaction (Promega GoTaQ) with specific primers for human factor IX, LC3 (autophagosome marker) and GAPDH as housekeeping gene. The reactions were run and analyzed on an ABI 7500 system.

qPCR primers:
GAPDH
F: AGATCCCTCCAAAATCAAGTGG
R: GGCAGAGATGATGACCCTTTT
Human factor IX
F: CAAGTATGGCATCTACACCAAAGTCT
R: GCAATAGCATCACAAATTTCACAAA
LC3
F: CGTCCTGGACAAGACCAAGT
R: ATTGCTGTCCCGAATGTCTC

### Effect of AAV5-miANG5 on the Plasma Lipid Levels in Dyslipidemic NHPs

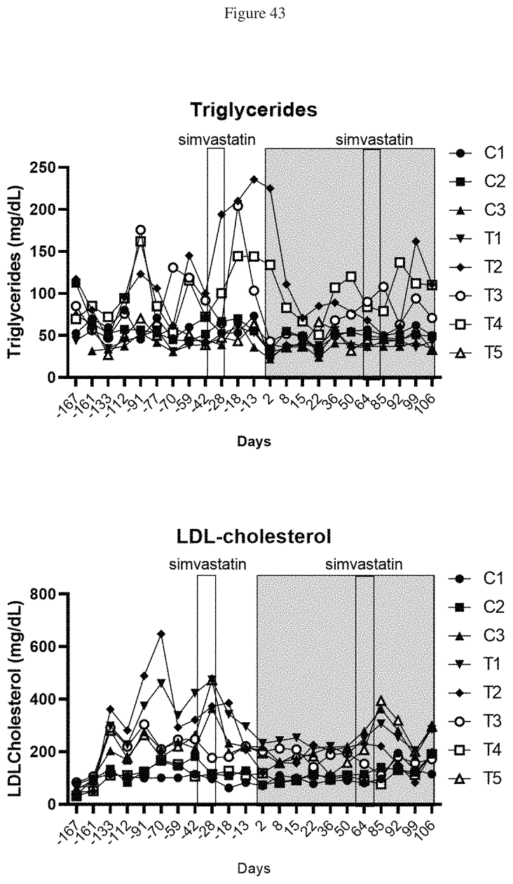

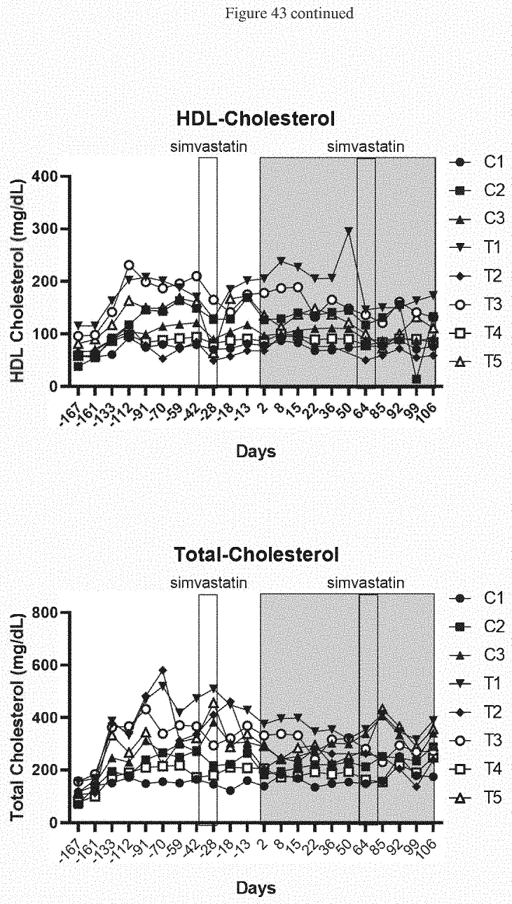

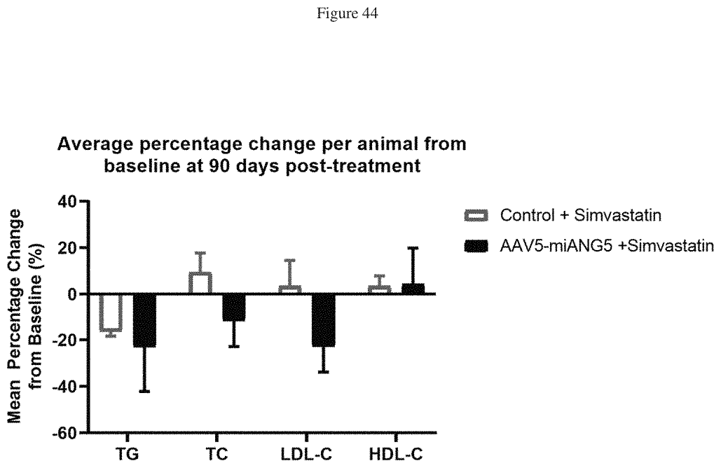

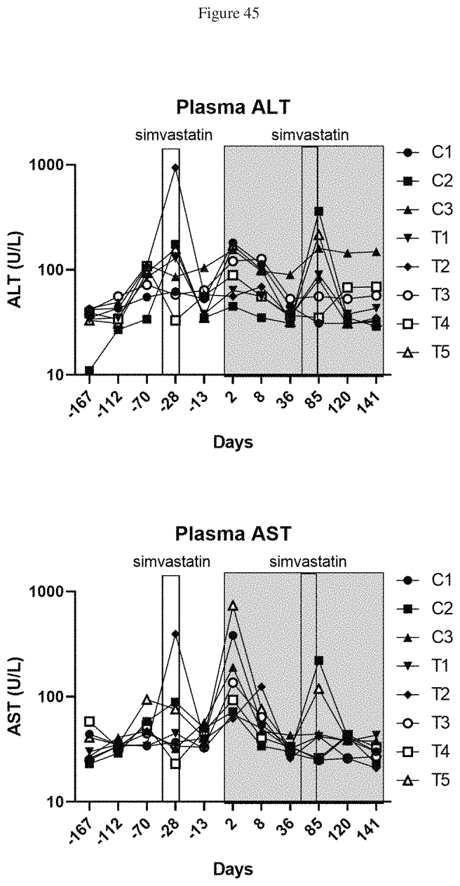

To assess the efficacy and safety, of AAV5-miANG5 in large animals, AAV5-miANG5 was IV injected in dyslipidemic Cynomolgus macaques. The NHPs received a high calorie diet for 167 days prior to dosing of the test material. The total cholesterol and triglyceride levels were elevated in all animals due to the high calorie diet. However, the response of each animal to the diet was highly variable. Prior to dosing the animals received Simvastatin to study their response to Simvastatin followed by a wash-out period. Three animals received the vehicle (formulation buffer) and 5 animals received AAV5-miANG5 at a dose of 1E+14 gc/kg intravenously. The objective of the study was to investigate the safety of silencing ANGPTL3 by AAV5-miANG5 and its effect on plasma lipid profile in co-administration with Simvastatin The results on triglyceride, LDL-C, HDL-C and total cholesterol levels showed that the levels are highly variable in the animals in response to the high calorie diet (FIG. 43). The Simvastatin treatment alone or in combination with AAV5-miANG5 did not show an (additive) effect on the lipid markers. Therefore, the lipid marker levels of 90 days pre- and post-treatment including Simvastatin treatment were used to calculate the change in baseline levels in the TG, TC, LDL-C and HDL-C (FIG. 44). Despite the variability a decrease seemed to be visible in the AAV5-miANG5 treated NHPs for triglyceride, total cholesterol, and LDL-C at 90 days post-dosing (FIG. 45), However, this conclusion was drawn cautiously due to the high variability. Treatment of dyslipidemic NHPs with Simvastatin and/or AAV5-miANG5 resulted in a transient increase in ALT/AST in some animals, which is an expected response towards statin and/or AAV-mediated gene therapy (FIG. 45). On target analyses (e.g. miRNA and ANGPTL3), histopathology and other additional lipid markers is measured.

### REFERENCES

Borel F, van Logtenstein R, Koornneef A, Maczuga P, Ritsema T, Petry H et al. In vivo knock-down of multidrug resistance transporters ABCC1 and ABCC2 by AAV-delivered shRNAs and by artificial miRNAs. J RNAi Gene Silencing 2011; 7: 434-442.
Chait A, Subramanian S. Hypertriglyceridemia: Pathophysiology, Role of Genetics, Consequences, and Treatment. 2019 Apr. 23. In: Feingold K R, Anawalt B, Boyce A, Chrousos G, Dungan K, Grossman A, Hershman J M, Kaltsas G, Koch C, Kopp P, Korbonits M, McLachlan R, Morley J E, New M, Perreault L, Purnell J, Rebar R, Singer F, Trence D L, Vinik A, Wilson D P, editors.
Ganda O P, Bhatt D L, Mason R P, Miller M, Boden W E. Unmet Need for Adjunctive Dyslipidemia Therapy in Hypertriglyceridemia Management. J Am Coll Cardiol. 2018 Jul. 17; 72(3):330-343.
Graham M J, Lee R G, Brandt T A, Tai U, Fu W, Peralta R, Yu R, Hurh E, Paz E, McEvoy B W, Baker B F, Pham N C, Digenio A, Hughes S G, Geary R S, Witztum J L, Crooke R M, Tsimikas S. Cardiovascular and Metabolic Effects of ANGPTL3 Antisense Oligonucleotides. N Engl J Med. 2017 Jul. 20; 377(3):222-232.
Kopin L, Lowenstein C. Dyslipidemia. Ann Intern Med. 2017 Dec. 5; 167(11):ITC81-ITC96.
Landgraf P, Rusu M, Sheridan R, Sewer A, lovino N, Aravin A, Pfeffer S, Rice A, Kamphorst A O, Landthaler M, Lin C, Socci N D, Hermida L, Fulci V, Chiaretti S, Foa R, Schliwka J, Fuchs U, Novosel A, Miller R U, Schermer B, Bissels U, Inman J, Phan Q, Chien M, Weir D B, Choksi R, De Vita G, Frezzetti D, Trompeter H I, Hornung V, Teng G, Hartmann G, Palkovits M, Di Lauro R, Wernet P, Macino G, Rogler C E, Nagle J W, Ju J, Papavasiliou F N, Benzing T, Lichter P, Tam W, Brownstein M J, Bosio A, Borkhardt A, Russo J J, Sander C, Zavolan M, Tuschl T. A mammalian microRNA expression atlas based on small RNA library sequencing. Cell. 2007 Jun. 29; 129(7):1401-14.
Lewis G F, Xiao C, Hegele R A. Hypertriglyceridemia in the genomic era: a new paradigm. Endocr Rev. 2015 February; 36(1):131-47.
Ling H, Burns T L, Hilleman D E. Novel strategies for managing dyslipidemia: treatment beyond statins. Postgrad Med. 2012 November; 124(6):43-54.
Minicocci I, Montali A, Robciuc M R, Quagliarini F, Censi V, Labbadia G, Gabiati C, Pigna G, Sepe M L, Pannozzo F, Lütjohann D, Fazio S, Jauhiainen M, Ehnholm C, Arca M. Mutations in the ANGPTL3 gene and familial combined hypolipidemia: a clinical and biochemical characterization. J Clin Endocrinol Metab. 2012 July; 97(7):E1266-75.
Nordestgaard B G, Nicholls S J, Langsted A, Ray K K, Tybjærg-Hansen A. Advances in lipid-lowering therapy through gene-silencing technologies. Nat Rev Cardiol. 2018 May; 15(5):261-272.
Olkkonen V M, Sinisalo J, Jauhiainen M. New medications targeting triglyceride-rich lipoproteins: Can inhibition of ANGPTL3 or apoC-III reduce the residual cardiovascular risk? Atherosclerosis. 2018 May; 272:27-32.
Palacio Rojas M, Prieto C, Bermúdez V, Garicano C, Núñez Nava T, Martínez M S, Salazar J, Rojas E, Pérez A, Marca Vicuña P, González Martinez N, Maldonado Parra S, Hoedebecke K, D′Addosio R, Cano C, Rojas J. Dyslipidemia: Genetics, lipoprotein lipase and HindIII polymorphism. Version 2. F1000Res. 2017 Nov. 30 [revised 2018 Jan. 1]; 6:2073.
Schleyer T, Hui S, Wang J, Zhang Z, Knapp K, Baker J, Chase M, Boggs R, Simpson R J Jr. Quantifying Unmet Need in Statin-Treated Hyperlipidemia Patients and the Potential Benefit of Further LDL-C Reduction Through an EHR-Based Retrospective Cohort Study. J Manag Care Spec Pharm. 2019 May; 25(5):544-554.
Toth P P, Patti A M, Giglio R V, Nikolic D, Castellino G, Rizzo M, Banach M. Management of Statin Intolerance in 2018: Still More Questions Than Answers. Am J Cardiovasc Drugs. 2018 June; 18(3):157-173.
Vroon D H and Israili Z (1990). Aminotransferases. In Walker H K, Hall W D, Hurst J W (Eds), Clinical Methods: The History, Physical, and Laboratory Examinations (3rd edition). Butterworths.
Zuker M. Mfold web server for nucleic acid folding and hybridization prediction. Nucleic Acids Res. 2003 Jul. 1; 31(13):3406-15.

Sequences
SEQ ID NO. 1
NA sequence human ANGPTL3 gene
AATGACAAACTGAAAAAATCTATTGTTTGTTATATATATA
ACAAAGAATTAGTATCCACAATATGTAAATAATTCCTAAA
ATTAGTCAGAAAGAGACAAACTTAAAAAGAGGGTAACAAG
GAGGGGAGCAAATTATGTACATAACCAGATGATTCGCAAA
GACGGCAACAGAGATGGCCAGCAAAACAAACTAGATATAT
ACTTGTCTATTAGATTTATCAACATTTTTTGCCTTTTTCA
TTAAAAGCATTTGTAAAAGGATATAGGAAAAGAGGAACTC
TCATATACTCCTGGCAGGGATGTAAATTGGTACAACCCTT
TTGAAGGACAATCTGACAAAAGCAATCGTAAGTTACAAGT
CAACATCTATGAATGTATATGAAAATATTTATATACATAC
ATCACCACCATAAAAGCATTTTCTATACATACTGTTTATA
ATTAGAAAATTGGAAACAAATGATTAAAAGGGGGCTGATT
AAATTAAGGTTCATCTATATAACAGGATTATGCAGCTATT
AAAAAGGACGTGGTAACTCTATAGACATTCATAGGAAAAT
AAATTTTAAAATACTAAGATCCTGAATGATATATATATCA
TGAGCTATTATACATAACAAGATCCCACTTGTGTTATAAA
AAATTATGTTTAGTCATTCAAAGGGTCTGGTATGATAGAC
CCAAAATGTTAATAGAGTCGAGATTTTTATTTTTTATAGG
TTTTTGAAATACCTGAATTTTCACAATAAGTACTTTGCAC
ATTAAAAATCTTAGCTGGGCATGGTGGCTCACGCTTGTAA
TCCCAGCACTCTGGGAGGCCAAGGTAGGCAGATCACCTGA
GGTCAGGAGTTCGAGACCAGTCTGGCCAACATGGTGAAAC
CCCGTCTCTACTAAAAATACAAAAATTAGCCAGGCTTGGT
TGGGGGTGCCTGTAATTCCAGATACTCGGGAAGCTGAGAC
AGGAGAATCGCTTGAACCCAGGAGGGGGAAGTTGCAGTGA
GCTGAGATCACGACGCTGCACTCCAGCCTGGGCAACAAAG
AGCAAAACTCCGTCTCAAAAATAAATAAAGAAAAAATCTT
TACATGTCCAAAGATACGGCTGTTCAACTAAAAAATATAT
ATGTATAAAACTTAACATGTTAATAGTGAACACACAAAAC
AGTAAGATAGATAAAATTATTCCTTCAAAGCTCACTTAAC
CTCTGGATCTACACTGTCCAAAAAGACGGTCTAATGAGAC
AATTGAGCACTTGATAGGTGAGTGGTTCTAACTGAGATAT
GTTCTCAGTATAAAACATACAATAGGATCTTCCTATACAA
CATTAATTAAAAAACAAACTATTGTAGTTAAAAAGGAAAA
AATTAGAGATACTATGTAAAAAAGAGCCAAAATACCTTGT
ATTTTATTTGAAAGACATATCTCCATAAGATTACACAACC
TCGTGTAGGATAAAGGACTTTGCTTTGCTTGGAATTTAAA
CAATTTAGGCTCTTAAATGTCCTAAAATTCTCTGTAGCTA
AGAAATTTTTATATTGGTTCCTAGGAACTAGGAATCCTTA
AATTAGGCCCTACATTTGCTTACAAGTTTATTTTCCTTGG
CATAAAATTTTTTAGTTTTTACATTACTGGTTATATTTGA
TCAGGGTTCTATTTAAATAGGCACAAGTTCAAGCAAAGAT
CAGATTCTGCTTTTAGCAGTGTGTACTCAGACAGGAAGTA
TTAAAAGGCAGGCAGAAAATCCTTTATAAAATTACTACTT
TCAATGCATTTTCCCACGTTGAAATGCTTCTGCAGTTTAT
AATTAGGCAAATTACTTTAATTATAATCAATAATGCTGTT
CAAATTACTATAAGAATTATACAGATATTTATACCAAGAG
ACAATATACTAGAAACCAAGACTACGTGACCATTACCTCT
ACTCTGTCAGTGTTATTTGTGAGAAATTGCACAAATTTTG
CAAAAAGTGTTAGTATCCTACTACAGTAGGATATAATATA
GAAGGAAATAATTTCATAAAGCCTGTCTTTGGTACTAGTG
CTCAGTTACTTTCATTAACTAAAAAAGGGGCTACTCTTCA
AATTCCCTTCTCTAAAAAGAATGTACTATATCAAAAGGGG
GTAAACACTACTACGTATACATTCTGCACTTAGAAATCCC
TATATGTTGATTTTATCATTCTCTTATTCAATAAATATTG
TTTCTACAATGTGTAAGGCATTACTGTACTAAAGCATTAT
AAGGAATATAAGTTAAAAACACATACAAATCTTGCCAATC
AACAGCTTATAGTGTAATAGGGGAGAGAAGCTGGCCCATC
TATATTCTCCCTCAACTAGCAAGTGGATGAAATATCAGGG
TCAATAGTTATAAGCCACAAAAGCTGACAGCTTAATTAAG
AGAAGTTTTGAAATATGTATTTCATGACCAATAATTACAA
CTGTAACTTTTCTATTTAAAGAAGGAGAAAATTTGAATTT
CTTCTCTAGCTCAACATACACTTCTATAATTCCATTACAT
GAACCAGAGTAAAGGGTAAGATGGAAATGAAGAATATTTT
CTTACCCTTTTGTGGTTCTATATTGGACACTTAAAAATCA
TACACAACCTAATCAAAAGATGTAATTCTTTAAAAAGGTA
CGAGACCAAAATTCAGAAAATCTAGACTATAACAAAATTT
CTCAATTTACATTATCTTAATATGCAATTAATTTTCACCA
GTAAAATACTATAGTATGGGTACAAATGCATTGATTAGTT
CTAATTACAAAAATGGCTAATATATAATACTGTGTAGTGT
TTATGATACATCAGATAATGTTCTAAGTGCTCTGAAAATA
TAAACTTTTAATCTTTTATACGACCCTATAAAATAGGTGT
TATTCTCACTGGAGAGATGAGAAAACAGGGGTTCAGAGAT
GTGAAGTAATTTGACCAAAGGTCACAAAGCTGAAGAATAT
GAAATCCGGGATTCTGATTCAGGCAGTCTTATTCCAGAAT
CATGCTCTTAACCACTATGGAATACTGCCTCTACTGTAAC
TATTATACCCAAAACCCTTAATCCTAAGTCATCAAAAGGA
AGAGCCTCTATTTTACACAATGAAGAGGCATTTCTAAGAA
TAGAAATTTAGGGACGAGCACAGTGGCTTACTCCTTTAAT
ATCAGCACTTTGAGAGGCTGATATGGGAGGTTCACTTGAA
GTCAGGAGTTCAAGGTCAGCTTGGGCAACATAGTGAAACA
CAGTCTCTACAAAATATTTAAAAATTAGCTGGGTGTGGTG
GCATGCATCTATAGTCTCAGCTACTTGGGAGACAGAGGGA
GGAGGCTTGCTCGAGCCCAGGAGTTCGTGGCTATAGTGAG
CTATGATCATGCCACTGCACTCCAGCCTGGACAACAGAGC
AAGACCCTGTCTCTAAAAAAGAAAAGAAATTTGGAAATGG
TTTATTTTGTATTAACAATTTATAATTTACACTGAAATTT
ATTATGATAAAACTTTTCCCTGTGTTAAAAAGCTATTAAC
TTTATGAAAAATTTCTTTTAGGTAAGGTTGATTATATATA
CCCACACACATACACAGGTTAAAAGTTAGTTTCATGTGAC
ATAATAACTAGCATTTTGAGCACTACCTGTTTGCCCAGCA
CTGTTCTAAGTGCTCTACATGTATTATTGTTAAATTATCA
TAACACTATGAATTATGTACTATAATTACCCCAGCTTTAC
AGATGAGGAGACTAATCCATGGGGAGGTTAAGTAACTTGT
CCAAGGCCAGACAGCTAGAGCCGGCTTTTGGACCCACACC
ACAGTCTGACTCCAGCACCCATATTCTTAACAATTTCACC
ATATTAATATGTCAAGATTAAGCAGTTTTAAAGGATGCTA
TTTTCTCACAAATTTCTTAATATGAACACTCAATAAGAAT
AATCACTAATATAAGCATTTAGTATTTTTTTAACACTAAG
TIGGAAGCATAGTGGAACATTTATTTTTAGAAATATTATT
AATTGGCTGGGCTCACGCTTGTAATCGGCTGGGCTCATGC
CTGTAAATTTTGGGAGGCCAAGGTAAAAGAATTGCTTGAG
CCCAGTATTTCCAGACCAGCATGGGCAATACATTAAGACA
TCATCTTTAAAAAAAAAATGTTATTAATCTCCTCTTTTTG
TTAAATGTATATTATCAAAATTGTTACTAAGCTAACAAAC
TTCAGAAAAACTTATGATGGGCAAGCTGCTTGTGACATTG
AAGGTATTTAAGATTCAATTCTAGTTTGGTCCTAGATGAC
CACATATCCATTGTTCCTTCAACGAGCACATGGTAAAGAG
CCTAGAACACAGAGACACAGAACACAGTGGAGAAAAGGGA
GTGAAATGTCTTTAATGACACTTACTATATATGGGATTTT
GTGACAATATACAAGGATGGTTAAGACATATAAGGTGATG
CAAAAAAACATATTAACAATTATAGTGACAAAAAATGAGG
AGCATATAATTATACATTGATTTATACAGAGTACCAGAGG
AACACAGCATTGAGAGCCGTAACACCACCTGAGGGAGTGG
AGAAAGGCTTCAGAGAGAAAGTGTTTTTTGGAATGGATCA
CTGTTTCCAAAAGAACTAAAGTACAGTTTGAGAAATGCAT
ACTTAATTCATTACTTTTTTCCCCTCAACTTTAATAATAA
ATTTACCCAACAAAAAAGTTTATTTTTGACTTGTAAATCT
CTTAAAATCATAAAAAAGTAAAATTAGCTTTTAAAAACAG
GTAGTCACCATAGCATTGAATGTGTAGTTTATAATACAGC
AAAGTTAAATACAATTTCAAATTACCTATTAAGTTAGTTG
CTCATTTCTTTGATTTCATTTAGCATTGATCTAACTCAAT
GTGGAAGAAGGTTACATTCGTGCAAGTTAACACGGCTTAA
TGATTAACTATGTTCACCTACCAACCTTACCTTTTCTGGG
CAAATATTGGTATATATAGAGTTAAGAAGTCTAGGTCTGC
TTCCAGAAGAAAACAGTTCCACGTTGCTTGAAATTGAAAA
TCAAGATAAAAATGTTCACAATTAAGCTCCTTCTTTTTAT
TGTTCCTCTAGTTATTTCCTCCAGAATTGATCAAGACAAT
TCATCATTTGATTCTCTATCTCCAGAGCCAAAATCAAGAT
TTGCTATGTTAGACGATGTAAAAATTTTAGCCAATGGCCT
CCTTCAGTTGGGACATGGTCTTAAAGACTTTGTCCATAAG
ACGAAGGGCCAAATTAATGACATATTTCAAAAACTCAACA
TATTTGATCAGTCTTTTTATGATCTATCGCTGCAAACCAG
TGAAATCAAAGAAGAAGAAAAGGAACTGAGAAGAACTACA
TATAAACTACAAGTCAAAAATGAAGAGGTAAAGAATATGT
CACTTGAACTCAACTCAAAACTTGAAAGCCTCCTAGAAGA
AAAAATTCTACTTCAACAAAAAGTGAAATATTTAGAAGAG
CAACTAACTAACTTAATTCAAAATCAACCTGAAACTCCAG
AACACCCAGAAGTAACTTCACTTAAAGTAAGTAGAAAATA
AAGAGGGTTCATGTTTATGTTTTCAATGTGGATCTTTTAA
AAAAAATATTTCTAAGGCATGCCATTTGAAATACTTTGTT
GCATTGTTGAAATACTTTTTTTTCCAAGAAAAATAATCTC
CAGAAAATAAAATTTCCTATTATAATTTCAAGTTAGTTTT
TTGTTTCCCTAATGTTATATATGAAAACACTGAAAATTTG
CATTTTATATGAAAATTACAAATCGGTTAAATTATACAAT
CTAGAACACTATGTCATTACACTATTGTAAATTACTGAAG
GTAAGTAAAAAGTTAAAAAAAATTTAAAACTATTCTCCAG
TGTTTAAAACAGATTAAATAATACAGTAAATGGAAAAGAT
TTATTCATATGAAAATATGCTGGGCTTTTTCTTTTAATTG
AAGTTCAGAAAATCAAATTTTAGAGATAGTACAATTTAAA
TAAAATGTTAAGGACAAAAATATGTGCTATTTGAAAGAAG
CATACAAGGGGAAGGAATTGCCAATATTCATTTTTCAAAT
CCATTATTAGTTTAAAAATTTAGATTATGATAGTGTTACA
GGAAATTAATAGAAAAGAAAGAGGAAAGCAACTTATAACC
AACCTACTCTCTATATCCAGACTTTTGTAGAAAAACAAGA
TAATAGCATCAAAGACCTTCTCCAGACCGTGGAAGACCAA
TATAAACAATTAAACCAACAGCATAGTCAAATAAAAGAAA
TAGAAAATCAGGTAAGTCAGTATTTTAATGGTATGTCCCA
TCTTTCACACAGGTCTGTAAAAACACTGAATCCTAAAATT
ATTTACAAGCTTTAACTGGATCATGAGTAAAATTATCACA
TCAGCATAACTGTTAAAATTGCAGGCTCTGAAGCTAATAA
ACTACCTGCATTTAAACCATGGCTCTAAAACTTTGTGTGA
CCTTGAATAAATTACTTCACCCCTTTATCTCTCAGTTTCC
TCACATATACTACAAAGATAATAACAGAACTTATAGGATT
ATTGTAAGAAAAAAAATTAATTCATAGCAGCCAATGTCAT
CTTACTAAAATTCAAATTAGATCATGTTTCTCTTTGCTCA
AAACCACACAATAGCTTTCCATTTCACTCATATTGGCTCT
TTAGACCAAGATTACCCAACCCTTCGTCATCTCACTGACT
TCACCTCCTCTACTCTAGTTATTCTGACCGCTTTACCAGT
ATTCAAACACATCAAACATACTGCCACCTCAAAGCCTTTG
CCCTTGTTGTTTCCTCTAACTGGAACGCTCTTCTGCCCTG
GTATCTACGTGGCCCACTCTCTGATTTCCCTTAGGGTCGT
TATCAAACAAAAAATTCCCAATGAAGACTTACAAGGTCAC
TTAACCAAAAATCACAACCGCCTGGTCCCATCCCTGAAAA
CTTCTACTTCCTTAGCTACTTTTCTCCTGCACACTCACCT
TTATTTAACATAACATAAATTTTAGTTATTTATCTCTTCT
ATTCCTGCACTAAAATGTAAGCTCTGTGAATACAGGGATT
TTTTCCATTATCTTCATATTTTCCATTATTTGTATATACT
CCAGAATATAGAATACTGTATGGCACACAGTAGGCATTTC
TGTTGAATTAATAAATGTAATGTCATATTCACACAGAAGC
GTGTGCTATGATTATTATTACTTGGATTACTAGAAATAGT
GTGCCTCATAATTAAAGGTCAACATTCAACAATGTAATTA
ATCTACAATGTAAACATCTGGTGAAGTGACAGAGGGAAGC
ACTTGTTTAGAAAAAAGCTATGTCAGAATCCATGTATTCT
AATATGCAGTACAATAGTTTAAAAATATTAATAATACTCT
CAAACAGCTATTCAAGAGGATTCAAAAAACATAATATAAA
CTCAGAGAAACTGGTAAACAAAATCATTTTCAAGAGATAT
AAAACAAATATTATTACCAATTTCCACTAAACAAACATAA
TGTTAGTAGTGCTGCTAAAAGGTTTTTTATCAACTACTTT
TGGTTTCCATACTTTCCTTCTTATGATGTTATTATTCTAA
ATTCTTTTCAATTATATCTTTTACTATGATTAAATGAACC
TGCTCCCCAAAGCAAAATGTTACTATAGTAATATACATTG
TGTCTAAAAATAAAAATGTGTGAAGAAACCAAAACAATGA
ATTTCTGAGTTGGAAGAAGAGTTAGATCATTTAACTTTCT
CATATTTAAATTAAAAAAACAAAACTCTAAAAATTTAAGT
AACTTTAAGATCACATAGTTACTTAGTAGAAAAGAGTAAT
ACCCAGCAAGCAAACTTTACAATAGATCCTTTTAAATAAG
GTCCTAGGAAATATCATTCATGCCAGCATCAAAAAACTAA
CACTAATAATGCAAGATATTATATATTCTGCTTTTCTTAC
TGTCAATGAGAAAAACTATCATTCAATAAATTGCAAACCC
AACACACTTAAATAAAAATAAAATGTTACTGCTAAACTAA
CGATAAACTACTGAATATATAGAAAGTAAGCAAACAAACT
TGCCAACCTGCCAACATCTACAGATATGTTTACAGGTCAA
AAATTATCAAATTATCAAGAAAGCCTGGTTCAAATTATGT
ATTATGTCTTTATCACAGGTCTGAAGATCAGTAAGACCTA
AAACTGAAAATTATTAAACTTAAAATCTGAACAGAATATC
AAATATATTTTATTCATATAAATAAAAGAATACATTACAA
TATTCTAAGCAAAGCAGTCTCTACTTTTGGCCTTGCTCTG
TTTTCCGACCAATGTCTGCTTTTTTGCCTTGCTTTATTTT
TTTATCTTATTAAATAATGTCCCTGATTAAATATTTTGAG
AACAGGTAATCTGTACAATCTGAATAACACTGTTTATCTA
AATATCAAACACCGTTATAACATTATGAACTGAAAGACAA
ACTGTACTTCTGACATCCTTACTCAGATTTCCCCTAATTG
TATATTCAGTATCATTTTAAAAAACAGATTTATATTCTTT
TATCAGCTCAGAAGGACTAGTATTCAAGAACCCACAGAAA
TTTCTCTATCTTCCAAGCCAAGAGCACCAAGAACTACTCC
CTTTCTTCAGTTGAATGAAATAAGAAATGTAAAACATGAT
GGTAAGACACTTTGGTGGGTTTCCTTCTTGAAGCTATTAT
TATCAAATTCCCTATTCTTAGGACTTGTTCTAGACTAAAA
GATAGTTAAGAGATATCCATCAAATACAATGTATCAACCT
AAACTGGATGCTGGGGTTCTTTTTACACCCTATAAAAGAC
ATACCTAAGACAATCAGAGAAATACAAATATGGACTTGAT
TATTAGATAATATAGAAGGTTTATTAATTTTCTTAGATGT
GATCATGGTATTGCAGTTTTAAAGGAGAACAATCTCCTGT
TTAAGAGATACATGCTGAAATATTTACGGAGTTAAAGGTC
ACTGGACTCCAGACTGGTGATAGAACAAGACTCTGTCTCT
AAAAAATAATTAATTTTTTAAAAGAAAATAGTTTGGTAAG
ATGATTCTTACATTCTTAAATAACACGCCATCTAAGAAAA
ATGCTTTAACATAAACATTACTGAAAAAATGCTACATTTG
CCACAACTTCATAAAATGTCAAGTGAAATCTCAAGCTCCA
AAGATATTATTCCTATTACTAAATCTGATGTAATAACATT
TTATTGATTCTAGGCATTCCTGCTGAATGTACCACCATTT
ATAACAGAGGTGAACATACAAGTGGCATGTATGCCATCAG
ACCCAGCAACTCTCAAGTTTTTCATGTCTACTGTGATGTT
ATATCAGGTAAAACCTGTCTAAGGAGAATAGACAGTAGTT
AGTTCAACTTACTCATTACGTATTAGGAAGATTAACCTGG
TTATCATTGTTTTATACATATATATATGAAATATATATGA
GTATTCGTATAAATATAATACTTTTACCTTGTTTATGTAT
TTACTCAATATTCTCCTTTTCCTCTAAAATAATCTGAAGT
GACTATTATCAATAAGTTTACTATGCCAAAATTCATTAAT
TGCCTTTCACTTAACTTTTGGGACCATAATAAATAATAAA
ATGTATTGCCATAACATTAATAAACTACCTTACAAAACCA
CCAATTAAAATCAAACAAACAAAAAAGTGTTATTTACATC
TGTCAACATAAATCTACTAAAAATACATGATTTCATTCAT
TATATTCAGGTAGTCCATGGACATTAATTCAACATCGAAT
AGATGGATCACAAAACTTCAATGAAACGTGGGAGAACTAC
AAATATGGTTTTGGGAGGCTTGATGGTAAGGGGACTACAT
TCAATCATTCATTCACTTGCTAATCTACAAATATTTACTG
AGAACCTCTTATGGACCAGGTATTAGGAAAAGTAGTAACG
AACGAGAAGCAGTCTCAGCCTTCATATAATTTATTATCAA
ACAATTACACATTTGTTAGTAAATTACACTTATTACAACT
GTTATTATTTGAATTATATTTATCACAATTACATGTCTGT
TCTTAAATATACTTATCACAATTTAATTCCACGGCTTACA
ATGATCATAACTATAATTATTAAAGACAATTTTGATTAAA
TGTTATGTCATAAGTAGTAACTGTTACAAATAAGCTGTGA
AAAGAACCACTCCTAGCATTAGTCACTCTATTCTCTCATT
AACGTTTTACATATCAATTAATTGGAAGTTAAAAGGACCA
GGAAACTCAGACATACAGTATACATTTTAAAATTTCAATT
ATTTAAATATAATATATAGAATGTATGGCTTATAATGAAT
TAGTTAACTCAATGCAAATTATTCTATTTTGATTACAAAT
AGTAAAATAAGCAAGATAAAATAACAGATGTTTAAAATCC
AAAAAGCACATACAAAAATCCATGAATGATGTCTAAGTAC
TCACTTATAAAGTAGAAGACATTCATTATTATATCAAATT
TTTAAATGCTCAGTACTATTTGACCATTTAAAAATTTTGT
ATTCAAACTACCAGTGAAAGCCCTACCTAGAAGGTATACT
CAGTGATAAGTTTTGTAGCTCCAAATCTTCTAATAGTGAG
TGTAACCCCAAAATAAAAGGCTGACAGGTAAGTCGAGAAT
ACTCACTTAATTCTGGTAAGAAAGCAACCCATTTGTACTT
GTATTTACCAGCAATCCTTAAAATGAAGCTTCCTACTAAC
TCAATAGCAATAAGACAATAGTGAATGTTTAATGAAAACA
GTATTTTATAAATACTTTAATAAAAAGGATTGTGATGAAG
AACAATCTATTTATATTTGTTATTTGTTTTTAATTCCAAT
AAAAATAATTTTTAAAATTACAGAAAAAAGTTATTAAGAA
CCATGCTTTTAAATTTAAAATGATTTTTTAAATTTATTCC
TGTCTTTTTCTACAAAGAAAGCATACATTAAGCAAATACC
AAAGGCCAGGTTTACATTTGAAGAAAGTGACATTATTATT
ACTCAAGTCTCTAGGAATACTTAACACATCTCTTGACTGT
ATATGGATGTTAATAAATAGCTGACAGTAAAGTTTATCCA
TATAAAGACTTGCAAATATTCCTCTACCAATGACGAGACT
TTAAAATATCTATAATAATGTAACACATTTCACTGGTGAA
ACATGTCTTGTCATATGCATTATAGAAAGGATAATCAGAC
TTTCAGTTATATTAATATTTTTAACATTTTTGTGCACATA
GCTATCTTCAATAAAATTGTTTTAAAAGGTATTATTTTAA
GATACACTAAAATGATCAAGGGATTCAAGACTAAACAACT
CAATTAGTIGCACCAATAAAAAACACTTAAAAAAACTGTC
AGTGTCCAACCTGTACTTAATAACTCACAGATTTTTAAAA
CTTTTCTTTTCAGGAGAATTTTGGTTGGGCCTAGAGAAGA
TATACTCCATAGTGAAGCAATCTAATTATGTTTTACGAAT
TGAGTTGGAAGACTGGAAAGACAACAAACATTATATTGAA
TATTCTTTTTACTTGGGAAATCACGAAACCAACTATACGC
TACATCTAGTTGCGATTACTGGCAATGTCCCCAATGCAAT
CCCGGAAAACAAAGATTTGGTGTTTTCTACTTGGGATCAC
AAAGCAAAAGGACACTTCAACTGTCCAGAGGGTTATTCAG
GTATCTTTTTCTGATACCAATACTTTATTTTCATATCTTC
AAAGTATCTTCCCACATTATTAGCTATTATCTGCAATGAC
AACTTTTAAAAATCCGAATCCCAAATAAGCGTTTTCTCTC
TAGACGAAAACCTCTTAACTATAATGAAAGTGTTCATTCT
AGTTCAATCAGGTATTTTACCTCTAATCTTCCTCAGATTT
TCTATTTTTTGGTAGTGTATAGATTATTTATACAGATTAT
TTAAAATTGGGACTTATACAGATTATTTAAAACTGGGATA
CATGCATCTAAAACACTGTAATATTTATAAGAAAGGAAGA
TAAACTTACGGGGAAATACAGTAACAGTAACTACATACGA
GTCTGTACCCATTAAATTGCATATCTATCTCCTTTAGGAG
GCTGGTGGTGGCATGATGAGTGTGGAGAAAACAACCTAAA
TGGTAAATATAACAAACCAAGAGCAAAATCTAAGCCAGAG
AGGAGAAGAGGATTATCTTGGAAGTCTCAAAATGGAAGGT
TATACTCTATAAAATCAACCAAAATGTTGATCCATCCAAC
AGATTCAGAAAGCTTTGAATGAACTGAGGCAAATTTAAAA
GGCAATAATTTAAACATTAACCTCATTCCAAGTTAATGTG
GTCTAATAATCTGGTATTAAATCCTTAAGAGAAAGCTTGA
GAAATAGATTTTTTTTATCTTAAAGTCACTGTCTATTTAA
GATTAAACATACAATCACATAACCTTAAAGAATACCGTTT
ACATTTCTCAATCAAAATTCTTATAATACTATTTGTTTTA
AATTTTGTGATGTGGGAATCAATTTTAGATGGTCACAATC
TAGATTATAATCAATAGGTGAACTTATTAAATAACTTTTC
TAAATAAAAAATTTAGAGACTTTTATTTTAAAAGGCATCA
TATGAGCTAATATCACAACTTTCCCAGTTTAAAAAACTAG
TACTCTTGTTAAAACTCTAAACTTGACTAAATACAGAGGA
CTGGTAATTGTACAGTTCTTAAATGTTGTAGTATTAATTT
CAAAACTAAAAATCGTCAGCACAGAGTATGTGTAAAAATC
TGTAATACAAATTTTTAAACTGATGCTTCATTTTGCTACA
AAATAATTTGGAGTAAATGTTTGATATGATTTATTTATGA
AACCTAATGAAGCAGAATTAAATACTGTATTAAAATAAGT
TCGCTGTCTTTAAACAAATGGAGATGACTACTAAGTCACA
TTGACTTTAACATGAGGTATCACTATACCTTATTTGTTAA
AATATATACTGTATACATTTTATATATTTTAACACTTAAT
ACTATGAAAACAAATAATTGTAAAGGAATCTTGTCAGATT
ACAGTAAGAATGAACATATTTGTGGCATCGAGTTAAAGTT
TATATTTCCCCTAAATATGCTGTGATTCTAATACATTCGT
GTAGGTTTTCAAGTAGAAATAAACCTCGTAACAAGTTACT
GAACGTTTAAACAGCCTGACAAGCATGTATATATGTTTAA
AATTCAATAAACAAAGACCCAGTCCCTAAATTATAGAAAT
TTAAATTATTCTTGCATGTTTATCGACATCACAACAGATC
CCTAAATCCCTAAATCCCTAAAGATTAGATACAAATTTTT
TACCACAGTATCACTTGTCAGAATTTATTTTTAAATATGA
TTTTTTAAAACTGCCAGTAAGAAATTTTAAATTAAACCCA
TTTGTTAAAGGATATAGTGCCCAAGTTATATGGTGACCTA
CCTTTGTCAATACTTAGCATTATGTATTICAAATTATCCA
ATATACATGTCATATATATTTTTATATGTCACATATATAA
AAGATATGTATGATCTATGTGAATCCTAAGTAAATATTTT
GTTCCAGAAAAGTACAAAATAATAAAGGTAAAAATAATCT
ATAATTTTCAGGACCACAGACTAAGCTGTCGAAATTAACG
CTGATTTTTTTAGGGCCAGAATACCAAAATGGCTCCTCTC
TTCCCCCAAAATTGGACAATTTCAAATGCAAAATAATTCA
TTATTTAATATATGAGTTGCTTCCTCTATTTGGTTTCCTT
AAAAAAAAAAAAAACTCTCATAGGACATGTTTCATTTTGT
TCCTTTCAGGAGTAGTAAATTAGACGTTTTCCCCATATAA
AGCTTTTTTCTACCAGAAAGATACTTCTGGTAGAAGAAGA
GAAAGGAGCTCTTTATGGTTCACACGACTGTCTCCTGTCC
TAACTACTTTGCTTAAAGTGCTCAAATTCCATCACTACTC
ACAGTTGTCTAATCTAAGTCTAATCCCCTTTGATCTCTCA
GACTACCTTCCCTTTTATCTCTCTACTACTTAATAATAAG
AATATCTTTTTTTCAAACTTGACCTTCATTTTGCTTTCAC
AATACTATACTCTCCATGGATTATCCCTTATCTGAATCCA
TCTTTATAACCCTATTCCTTTCTCATATTTAGTACTGTGG
GCCAATGGACAACCTTCAATCATCTTTTCTACACTGACCC
TCAGACATTCTATCTGCTCTCACGGACTCCTTTATTTACC
ATGAATAAAGTTCCAAAATCTACATATTCATCCCAAGTCT
CTTTCCAGTTCCCCTTCTTACATTGCCTATTTGCCATTTC
TCCCTTCAATACCCTATACTTCACTCAAATTCAACATACC
AAAAATAAAAGGCCAGGCACGGTGGCTCACACCTGTAATC
CCAGGACTTTGGGAGGCTGAGGCAGGTGGATCACCTGAGG
TCAGGAGTCTGACCAGCCTGACCAATATGGTGAAACCCCG
TCTCTACCTAAAATACAAAAATTAGCCAGGCGTGGTGGCA
TGTGCCTACAGTCCCAGCTACTCAAGAGGCTGAGACAGGA
GAATCGCTTGAACCCAGGAGGCGGAGGTTGCAGTGAGCTG
AGATCACACCAATGCACTGGGTGACAGAACAAGACTGACT
CAAAAAAAAATAAATAACAAATTCCCCAGCCCCTTACTGC
TACTGCTATCCCTTTCTACCCACCTTTCCCTCCTTTATAC
TCTTTCACACCATCTTCCTCACTTCTTTATATCCATTAAT
ATGACCAGCATGTTCCCAGTCACAGAAGCCTGGAACCCGG
AAGACATCTCTGGCTTTTCACTCAACTTTGTAAACTACCT
CTTTTGTATCATAAGCCACCAAGTTCAATACAATCTTCTC
TTGAAACGTCTCTTAATCTTATAAGCTTTCTTCCCCAAAG
ACTGTCTTTAACTTCAGTGCTAGATTATATAAGT
NA sequence human ANGPTL3 mRNA
SEQ ID NO. 2
AGAAGAAAACAGTTCCACGTTGCTTGAAATTGAAAATCAA
GATAAAAATGTTCACAATTAAGCTCCTTCTTTTTATTGTT
CCTCTAGTTATTTCCTCCAGAATTGATCAAGACAATTCAT
CATTTGATTCTCTATCTCCAGAGCCAAAATCAAGATTTGC
TATGTTAGACGATGTAAAAATTTTAGCCAATGGCCTCCTT
CAGTTGGGACATGGTCTTAAAGACTTTGTCCATAAGACGA
AGGGCCAAATTAATGACATATTTCAAAAACTCAACATATT
TGATCAGTCTTTTTATGATCTATCGCTGCAAACCAGTGAA
ATCAAAGAAGAAGAAAAGGAACTGAGAAGAACTACATATA
AACTACAAGTCAAAAATGAAGAGGTAAAGAATATGTCACT
TGAACTCAACTCAAAACTTGAAAGCCTCCTAGAAGAAAAA
ATTCTACTTCAACAAAAAGTGAAATATTTAGAAGAGCAAC
TAACTAACTTAATTCAAAATCAACCTGAAACTCCAGAACA
CCCAGAAGTAACTTCACTTAAAACTTTTGTAGAAAAACAA
GATAATAGCATCAAAGACCTTCTCCAGACCGTGGAAGACC
AATATAAACAATTAAACCAACAGCATAGTCAAATAAAAGA
AATAGAAAATCAGCTCAGAAGGACTAGTATTCAAGAACCC
ACAGAAATTTCTCTATCTTCCAAGCCAAGAGCACCAAGAA
CTACTCCCTTTCTTCAGTTGAATGAAATAAGAAATGTAAA
ACATGATGGCATTCCTGCTGAATGTACCACCATTTATAAC
AGAGGTGAACATACAAGTGGCATGTATGCCATCAGACCCA
GCAACTCTCAAGTTTTTCATGTCTACTGTGATGTTATATC
AGGTAGTCCATGGACATTAATTCAACATCGAATAGATGGA
TCACAAAACTTCAATGAAACGTGGGAGAACTACAAATATG
GTTTTGGGAGGCTTGATGGAGAATTTTGGTTGGGCCTAGA
GAAGATATACTCCATAGTGAAGCAATCTAATTATGTTTTA
CGAATTGAGTTGGAAGACTGGAAAGACAACAAACATTATA
TTGAATATTCTTTTTACTTGGGAAATCACGAAACCAACTA
TACGCTACATCTAGTTGCGATTACTGGCAATGTCCCCAAT
GCAATCCCGGAAAACAAAGATTTGGTGTTTTCTACTTGGG
ATCACAAAGCAAAAGGACACTTCAACTGTCCAGAGGGTTA
TTCAGGAGGCTGGTGGTGGCATGATGAGTGTGGAGAAAAC
AACCTAAATGGTAAATATAACAAACCAAGAGCAAAATCTA
AGCCAGAGAGGAGAAGAGGATTATCTTGGAAGTCTCAAAA
TGGAAGGTTATACTCTATAAAATCAACCAAAATGTTGATC
CATCCAACAGATTCAGAAAGCTTTGAATGAACTGAGGCAA
ATTTAAAAGGCAATAATTTAAACATTAACCTCATTCCAAG
TTAATGTGGTCTAATAATCTGGTATTAAATCCTTAAGAGA
AAGCTTGAGAAATAGATTTTTTTTATCTTAAAGTCACTGT
CTATTTAAGATTAAACATACAATCACATAACCTTAAAGAA
TACCGTTTACATTTCTCAATCAAAATTCTTATAATACTAT
TTGTTTTAAATTTTGTGATGTGGGAATCAATTTTAGATGG
TCACAATCTAGATTATAATCAATAGGTGAACTTATTAAAT
AACTTTTCTAAATAAAAAATTTAGAGACTTTTATTTTAAA
AGGCATCATATGAGCTAATATCACAACTTTCCCAGTTTAA
AAAACTAGTACTCTTGTTAAAACTCTAAACTTGACTAAAT
ACAGAGGACTGGTAATTGTACAGTTCTTAAATGTTGTAGT
ATTAATTTCAAAACTAAAAATCGTCAGCACAGAGTATGTG
TAAAAATCTGTAATACAAATTTTTAAACTGATGCTTCATT
TTGCTACAAAATAATTTGGAGTAAATGTTTGATATGATTT
ATTTATGAAACCTAATGAAGCAGAATTAAATACTGTATTA
AAATAAGTTCGCTGTCTTTAAACAAATGGAGATGACTACT
AAGTCACATTGACTTTAACATGAGGTATCACTATACCTTA
TTTGTTAAAATATATACTGTATACATTTTATATATTTTAA
CACTTAATACTATGAAAACAAATAATTGTAAAGGAATCTT
GTCAGATTACAGTAAGAATGAACATATTTGTGGCATCGAG
TTAAAGTTTATATTTCCCCTAAATATGCTGTGATTCTAAT
ACATTCGTGTAGGTTTTCAAGTAGAAATAAACCTCGTAAC
AAGTTACTGAACGTTTAAACAGCCTGACAAGCATGTATAT
ATGTTTAAAATTCAATAAACAAAGACCCAGTCCCTAAATT
ATAGAAATTTAAATTATTCTTGCATGTTTATCGACATCAC
AACAGATCCCTAAATCCCTAAATCCCTAAAGATTAGATAC
AAATTTTTTACCACAGTATCACTTGTCAGAATTTATTTTT
AAATATGATTTTTTAAAACTGCCAGTAAGAAATTTTAAAT
TAAACCCATTTGTTAAAGGATATAGTGCCCAAGTTATATG
GTGACCTACCTTTGTCAATACTTAGCATTATGTATTTCAA
ATTATCCAATATACATGTCATATATATTTTTATATGTCAC
ATATATAAAAGATATGTATGATCTATGTGAATCCTAAGTA
AATATTTTGTTCCAGAAAAGTACAAAATAATAAAGGTAAA
AATAATCTATAATTTTCAGGACCACAGACTAAGCTGTCGA
AATTAACGCTGATTTTTTTAGGGCCAGAATACCAAAATGG
CTCCTCTCTTCCCCCAAAATTGGACAATTTCAAATGCAAA
ATAATTCATTATTTAATATATGAGTTGCTTCCTCTATTTG
GTTTCC
HCR-hAAT promoter sequence
SEQ ID NO. 85
GGCTCAGAGGCACACAGGAGTTTCTGGGCTCACCCTGCCC
CCTTCCAACCCCTCAGTTCCCATCCTCCAGCAGCTGTTTG
TGTGCTGCCTCTGAAGTCCACACTGAACAAACTTCAGCCT
ACTCATGTCCCTAAAATGGGCAAACATTGCAAGCAGCAAA
CAGCAAACACACAGCCCTCCCTGCCTGCTGACCTTGGAGC
TGGGGCAGAGGTCAGAGACCTCTCTGGGCCCATGCCACCT
CCAACATCCACTCGACCCCTTGGAATTTCGGTGGAGAGGA
GCAGAGGTTGTCCTGGCGTGGTTTAGGTAGTGTGAGAGGA
TCTTGCTACCAGTGGAACAGCCACTAAGGATTCTGCAGTG
AGAGCAGAGGGCCAGCTAAGTGGTACTCTCCCAGAGACTG
TCTGACTCACGCCACCCCCTCCACCTTGGACACAGGACGC
TGTGGTTTCTGAGCCAGGTACAATGACTCCTTTCGGTAAG
TGCAGTGGAAGCTGTACACTGCCCAGGCAAAGCGTCCGGG
CAGCGTAGGCGGGCGACTCAGATCCCAGCCAGTGGACTTA
GCCCCTGTTTGCTCCTCCGATAACTGGGGTGACCTTGGTT
AATATTCACCAGCAGCCTCCCCCGTTGCCCCTCTGGATCC
ACTGCTTAAATACGGACGAGGACAGGGCCCTGTCTCCTCA
GCTTCAGGCACCACCACTGACCTGGGACAGTGAATCGTAA
GT
Q1 promoter sequence
SEQ ID NO. 80
AAGCAAATAT TTGTGGTTAT GGATTAACTC GAACTGTTTG
CCCACTCTATTTGCCCGGCG CCCTTTGGAC CTTTTGCAAT
CCTGGAGCAA ACAGCAAACACGGACTTAGC CCCTGTTTGC
TCCTCCGATA ACTGGGGTGA CCTTGGTTAATATTCACCAG
CAGCCTCGGG CATATAAAAC AGGGGCAAGG CACAGACTCA
TAGCAGAGCA ATCACCACCA AGCCTGGAAT AACTGCAGCC
ACC
Q1-prime promoter sequence
SEQ ID NO. 81
AAGCAAATAT TTGTGGTTAT GGATTAACTC GAACTGTTTG
CCCACTCTAT TTGCCCGGCG CCCTTTGGAC CTTTTGCAAT
CCTGGAGCAA ACAGCAAACA CGACTCAGAT CCCAGCCAGT
GGACTTAGCC CCTGTTTGCT CCTCCGATAA CTGGGGTGAC
CTTGGTTAAT ATTCACCAGC AGCCTCCCCC GTTGCCCCTC
TGGGGCATAT AAAACAGGGG CAAGGCACAG ACTCATAGCA
GAGCAATCAC CACCAAGCCT GGAATAACTG CAGCCACC
C14 promoter sequence
SEQ ID NO. 82
TTAATATTTAACATCCTAGCACAGCTTCACTTCCAGGTAT
GACCTTTGAACCTCTTCTAGAAGGGTAATTATTAACCTAG
CTAGGTATGACCTTCGAACCTCTTCTAGAAGTGAAGCTGG
GCATATAAACAGGGGCAAGGCACAGACTCATAGCAGAGCA
ATTACCACCAAGCCTGGAATAGCTGCAGCCACC
C16 promoter sequence
SEQ ID NO. 83
GGTTAATAATTACCCTTCTAGGATTGAGTCACTTCTAGAA
GCTGGACTTTGGACTCATCCTAGAAGTCACTTCCTCTTTT
TTACCTAGAAGAGGTTCAAAGGTCATACCTAGCATAGCTT
CACTTCTAGAAGGGTAATTATTAACCGGGCATATAAAACA
GGGGCAAGGCACAGACTCATAGCAGAGCAATCACCACCAG
GCCTGGAATAACTGCAGCCACC
promoter sequence
SEQ ID NO. 84
AAGCAAATATTTGTGGTTATGGATTAACTCGAACTGTTTG
CCCACTCTATTTGCCCGGCGCCCTTTGGACCTTTTGCAAT
CCTGGAGCAAACAGCAAACACGGACTTAGCCCCTGTTTGC
TCCTCCGATAACTGGGGTGACCTTGGTTAATATTCACCAG
CAGCCTCATGAGCGGAAGTGGGTCTCAACCACTATAAATC
CTCTCTGTGCCCGTCCGGAGCTGGTGAGGACAGCCACC
promoter sequence
SEQ ID NO. 85
TAAAGCAAATATTTGTGGTTATGGATTAACTCGAACTTCT
AGAAGCTGTTTGCCCACTCTATTTGCCCATCCTAGGTAGG
CGCCCTTTGGACCTTTTGCAATCCTGGCTTCTAGAAGAGC
AAACAGCAAACACATCCTAGGTAGGACTTAGCCCCTGTTT
GCTCCTCCGATAACTGGGGTGACCTTGGTTAATATTCACC
AGCAGCCTCAT
promoter sequence
SEQ ID NO. 86
AAGCAAATATTTGTGGTTATGGATTAACTCGAACTGTTTG
CCCACTCTATTTGCCCGGCGCCCTTTGGACCTTTTGCAAT
CCTGGAGCAAACAGCAAACACGGACTTAGCCCCTGTTTGC
TCCTCCGATAACTGGGGTGACCTTGGTTAATATTCACCAG
CAGCCTCGGGCATATAAAACAGGGGCAAGGCACAGACTCA
TAGCAGAGCAATCACCACCAAGCCTGGAATAACTGCAGCC
ACC
promoter sequence
SEQ ID NO. 87
AAGCAAATATTTGTGGTTATGGATTAACTCGAACTGTTTG
CCCACTCTATTTGCCCGGCGCCCTTTGGACCTTTTGCAAT
CCTGGAGCAAACAGCAAACACGCCCCTGTTTGCTCCTCCG
ATAACTGGGGTGACCTTGGTTAATATTCACCAGGGCATAT
AAAACAGGGGCAAGGCACAGACTCATAGCAGAGCAATCAC
CACCAAGCCTGGAATAACTGCAGCCACC
SV40 poly A sequence
SEQ ID NO. 88
TGCTTTATTTGTGAAATTTGTGATGCTATTGCTTTATTTG
TAACCATTATAAGCTGCAATAAACAAGTTAACAACAACAA
TTGCATTCATTTTATGTTTCAGGTTCAGGGGGAGGTGTGG
GAGGTTTTTTAAA
Macaca fascicularis, the NCBI accession
number XM_005543185.2,
PREDICTED: Macaca fascicularis
angiopoietin like 3 (ANGPTL3), mRNA
SEQ ID NO. 89
TACAATTTCAAATTACCTATTAAGTTAGTTGCTCATTTCT
TTGATTTCATTTAGCATTGATGTAACTCAATGTGGAAGAA
GGTTACATTCGTGCAAGTTAACATGGCTTAATGATTAACT
ATATTCACCTGCCAACCTTGCCTTTTCTGTGGCAAATATT
GGTATATATAGAGTTAAGAAGTCTAGGTCTGCTTCCAGAA
GAACACAGTTCCACGCTGCTTGAAATTGAAAATCAGGATA
AAAATGTTCACAATTAAGCTCCTTCTTTTTATTGTTCCTC
TAGTTATTTCCTCCAGAATTGACCAAGACAATTCATCATT
TGATTCTGTATCTCCAGAGCCAAAATCAAGATTTGCTATG
TTAGACGATGTAAAAATTTTAGCCAATGGCCTCCTTCAGT
TGGGACATGGTCTTAAAGACTTTGTCCATAAGACTAAGGG
CCAAATTAATGACATATTTCAAAAACTCAACATATTTGAT
CAGTCTTTTTATGATCTATCACTGCAAACCAGTGAAATCA
AAGAAGAAGAAAAGGAACTGAGAAGAACTACATATAAACT
ACAAGTCAAAAATGAAGAGGTAAAGAATATGTCACTTGAA
CTCAACTCAAAACTTGAAAGCCTCCTAGAAGAAAAAATTC
TACTTCAACAAAAAGTGAAATATTTAGAAGAGCAACTAAC
TAACTTAATTCAAAATCAACCTGCAACTCCAGAACATCCA
GAAGTAACTTCACTTAAAAGTTTTGTAGAAAAACAAGATA
ATAGCATCAAAGACCTTCTCCAGACTGTGGAAGAACAATA
TAAGCAATTAAACCAACAGCATAGTCAAATAAAAGAAATA
GAAAATCAGCTCAGAATGACTAATATTCAAGAACCCACAG
AAATTTCTCTATCTTCCAAGCCAAGAGCACCAAGAACTAC
TCCCTTTCTTCAGCTGAATGAAATAAGAAATGTAAAACAT
GATGGCATTCCTGCTGATTGTACCACCATTTACAATAGAG
GTGAACATATAAGTGGCACGTATGCCATCAGACCCAGCAA
CTCTCAAGTTTTTCATGTCTACTGTGATGTTGTATCAGGT
AGTCCATGGACATTAATTCAACATCGAATAGATGGATCAC
AAAACTTCAATGAAACGTGGGAGAACTACAAATATGGTTT
CGGGAGGCTTGATGGAGAATTCTGGTTGGGCCTAGAGAAG
ATATACTCCATAGTGAAGCAATCTAATTACGTTTTACGAA
TTGAGTTGGAAGACTGGAAAGACAACAAACATTATATTGA
ATATTCTTTTTACTTGGGAAATCACGAAACCAACTATACG
CTACATGTAGTTAAGATTACTGGCAATGTCCCCAATGCAA
TCCCGGAAAACAAAGATTTGGTGTTTTCTACTTGGGATCA
CAAAGCAAAAGGACACTTCAGCTGTCCAGAGAGTTATTCA
GGAGGCTGGTGGTGGCATGATGAGTGTGGAGAAAACAACC
TAAATGGTAAATATAACAAACCAAGAACAAAATCTAAGCC
AGAGCGGAGAAGAGGATTATCCTGGAAGTCTCAAAATGGA
AGGTTATACTCTATAAAATCAACCAAAATGTTGATCCATC
CAACAGATTCAGAAAGCTTTGAATGAACTGAGGCAAATTT
AAAAGGCAATAAATTAAACATTAAACTCATTCCAAGTTAA
TGTGGTTTAATAATCTGGTATTAAATCCTTAAGAGAAGGC
TTGAGAAATAGATTTTTTTATCTTAAAGTCACTGTCAATT
TAAGATTAAACATACAATCACATAACCTTAAAGAATACCA
TTTACATTTCTCAATCAAAATTCTTACAACACTATTTGTT
TTATATTTTGTGATGTGGGAATCAATTTTAGATGGTCGCA
ATCTAAATTATAATCAACAGGTGAACTTACTAAATAACTT
TTCTAAATAAAAAACTTAGAGACTTTAATTTTAAAAGTCA
TCATATGAGCTAATATCACAATTTTCCCAGTTTAAAAAAC
TAGTTTTCTTGTTAAAACTCTAAACTTGACTAAATAAAGA
GGACTGATAATTATACAGTTCTTAAATTTGTTGTAATATT
AATTTCAAAACTAAAAATTGTCAGCACAGAGTATGTGTAA
AAATCTGTAATATAAATTTTTAAACTGATGCCTCATTTTG
CTACAAAATAATCTGGAGTAAATTTTTGATAGGATTTATT
TATGAAACCTAATGAAGCAGGATTAAATACTGTATTAAAA
TAGGTTCGCTGTCTTTTAAACAAATGGAGATGATGATTAC
TAAGTCACATTGACTTTAATATGAGGTATCACTATACCTT
AACATATTTGTTAAAACGTATACTGTATACATTTTGTGTA
TTTTAATACTTAATACTATGAAAACAAGTAATTGTAAACG
TATCTTGTCAGATTACAATAGGAATGAACATATTGGTGAC
ATCGAGTTAAAGTTTATATTTCCCCTAAATATGCTGCGAT
TCCAATATATTCATGTAGGTTTTCAAGCAGAAATAAACCT
TGTAACAAGTTACTGACTAAACAGCCTGACAAGTATGTAT
ATATGTTTAAAATTCAATAAATAAAGACCCAGTCTTCTAA
ATTATAAAAATTTAAATTAGTCTTGCACAAATTAAATTAT
TCATCACAAAAGATGTATTGTTATTTTTAAGTCATTTAAG
CCCTAAATCCCTAAAGATTAGATATAAATTTTTTTTGCCA
GAGTATAAATTGTCAGAATTTATTTTTAAATATATTTTTT
AAAACTACCAGTAAGAAATTTTAAATTAAACCCATTTGTT
AAAGGATATAGTGCCCAAGTTATACGGTGACCTACCTTTG
TCAATATTTAGCATTATGTATTTCAAATTATCCAATATAC
ATGTCATATATATTTTTATATGTTGCATATATAAAAGATA
TACACGATTTATGTGAATCCTATGTAAATATTTTGTTCCA
GAAAAGTACAAAATAATAAAGGTAAAAATAATCCA
NA sequence mouse Angptl3 mRNA
SEQ ID NO. 90
ACAGGAGGGAGAAGTTCCAAATTGCTTAAAATTGAATAAT
TGAGACAAAAAATGCACACAATTAAATTATTCCTTTTTGT
TGTTCCTTTAGTAATTGCATCCAGAGTGGATCCAGACCTT
TCATCATTTGATTCTGCACCTTCAGAGCCAAAATCAAGAT
TTGCTATGTTGGATGATGTCAAAATTTTAGCGAATGGCCT
CCTGCAGCTGGGTCATGGACTTAAAGATTTTGTCCATAAG
ACTAAGGGACAAATTAACGACATATTICAGAAGCTCAACA
TATTTGATCAGTCTTTTTATGACCTATCACTTCGAACCAA
TGAAATCAAAGAAGAGGAAAAGGAGCTAAGAAGAACTACA
TCTACACTACAAGTTAAAAACGAGGAGGTGAAGAACATGT
CAGTAGAACTGAACTCAAAGCTTGAGAGTCTGCTGGAAGA
GAAGACAGCCCTTCAACACAAGGTCAGGGCTTTGGAGGAG
CAGCTAACCAACTTAATTCTAAGCCCAGCTGGGGCTCAGG
AGCACCCAGAAGTAACATCACTCAAAAGTTTTGTAGAACA
GCAAGACAACAGCATAAGAGAACTCCTCCAGAGTGTGGAA
GAACAGTATAAACAATTAAGTCAACAGCACATGCAGATAA
AAGAAATAGAAAAGCAGCTCAGAAAGACTGGTATTCAAGA
ACCCTCAGAAAATTCTCTTTCTTCTAAATCAAGAGCACCA
AGAACTACTCCCCCTCTTCAACTGAACGAAACAGAAAATA
CAGAACAAGATGACCTTCCTGCCGACTGCTCTGCCGTTTA
TAACAGAGGCGAACATACAAGTGGCGTGTACACTATTAAA
CCAAGAAACTCCCAAGGGTTTAATGTCTACTGTGATACCC
AATCAGGCAGTCCATGGACATTAATTCAACACCGGAAAGA
TGGCTCACAGGACTTCAACGAAACATGGGAAAACTACGAA
AAGGGCTTTGGGAGGCTCGATGGAGAATTTTGGTTGGGCC
TAGAGAAGATCTATGCTATAGTCCAACAGTCTAACTACAT
TTTACGACTCGAGCTACAAGACTGGAAAGACAGCAAGCAC
TACGTTGAATACTCCTTTCACCTGGGCAGTCACGAAACCA
ACTACACGCTACATGTGGCTGAGATTGCTGGCAATATCCC
TGGGGCCCTCCCAGAGCACACAGACCTGATGTTTTCTACA
TGGAATCACAGAGCAAAGGGACAGCTCTACTGTCCAGAAA
GTTACTCAGGTGGCTGGTGGTGGAATGACATATGTGGAGA
AAACAACCTAAATGGAAAATACAACAAACCCAGAACCAAA
TCCAGACCAGAGAGAAGAAGAGGGATCTACTGGAGACCTC
AGAGCAGAAAGCTCTATGCTATCAAATCATCCAAAATGAT
GCTCCAGCCCACCACCTAAGAAGCTTCAACTGAACTGAGA
CAAAATAAAAGATCAATAAATTAAATATTAAAGTCCTCCC
GATCACTGTAGTAATCTGGTATTAAAATTTTAATGGAAAG
CTTGAGAATTGAATTTCAATTAGGTTTAAACTCATTGTTA
AGATCAGATATCACCGAATCAACGTAAACAAAATTTATCT
TTTTCAATC
NA sequence rat Angptl3 mRNA
SEQ ID NO. 91
GACGTTCCAAATTGCTTGAAATTGAATAATTGAAACAAAA
ATGCACACAATTAAGCTGCTCCTTTTTGTTGTTCCTCTAG
TAATTTCGTCCAGAGTTGATCCAGACCTTTCGCCATTTGA
TTCTGTACCGTCAGAGCCAAAATCAAGATTTGCTATGTTG
GATGATGTCAAAATTTTAGCCAATGGCCTCCTGCAGCTGG
GTCATGGTCTTAAAGATTTTGTCCGTGTTTTTATGACCTA
TCACTTCAAACCAATGAAATCAAAGAAGAGGAAAAGGAGC
TAAGAAGAACCACATCTAAACTACAAGTTAAAAACGAAGA
GGTGAAGAATATGTCACTTGAACTGAACTCAAAGCTTGAA
AGTCTACTGGAGGAGAAGATGGCGCTCCAACACAGAGTCA
GGGCTTTGGAGGAACAGCTGACCAGCTTGGTTCAGAACCC
GCCTGGGGCTCGGGAGCACCCAGAGGTAACGTCACTTAAA
AGTTTTGTAGAACAGCAAGATAACAGCATAAGAGAACTCC
TCCAGAGTGTGGAAGAACAATATAAACAACTAAGTCAACA
GCACATTCAGATAAAAGAAATAGAAAATCAGCTCAGAAAG
ACTGGCATTCAAGAACCCACTGAAAATTCTCTTTATTCTA
AACCAAGAGCACCAAGAACTACTCCCCCTCTTCATCTGAA
GGAAGCAAAAAATATAGAACAAGATGATCTGCCTGCTGAC
TGCTCTGCCATTTATAACAGAGGTGAACATACAAGTGGCG
TGTATACTATTAGACCAAGCAGCTCTCAAGTGTTTAATGT
CTACTGTGACACCCAATCAGGCACTCCACGGACATTAATT
CAACACCGGAAAGATGGCTCTCAAAACTTCAACCAAACGT
GGGAAAACTACGAAAAGGGTTTTGGGAGGCTTGATGGTAA
AGTGATTTCCTTGCATCACTCACTTATCTGTTGATTTAAT
AGTATTAGTTGGGTGTGTTGACACAGGCCTGAGACCATAG
CGCTTTTGGGCAAGGGGGGAGGAGGAGCAGCAGGTGAATT
GAAAGTTCAAGACCAGTCTGGGCCACACATTGATACTCCT
TCTCGACATTAAGAATTATAAATTAAGCAGCAATTATAAA
ATGGGCTGTGGAAATGTAACAATAAGCAAAAGCAGACCCC
AGTCTTCATAAAACTGATTGGTAAATATTATCCATGATAG
CAACTGCAATGATCTCATTGTACTTATCACTACTGCATGC
CTGCAGTATGCTTGTTGAAACTTAATTCTATAGTTCATGG
TTATCATAAGTCTTATTAAGGAACATAGTATACGCCATTG
GCTCTAGTGAGGGGCCATGCTACAAATGAGCTGCAAAGAT
AGCAGTATAGAGCTCTTTCAGTGATATCCTAAGCACAACG
TAACACAGGTGAAATGGGCTGGAGGCACAGTIGTGGTGGA
ACACGCGGCCAGCAGGACACTGGGACTGATCCCCAGCAGC
ACAAAGAAAGTGATAGGAACACAGAGCGAGAGTTAGAAGG
GACAGGGTCACCGTCAGAGATACGGTGTCTAACTCCTGCA
ACCCTACCTGTAATTATTCCATATTATAAACATATACTAT
ATAACTGTGGGTCTCTGCATGTTCTAGAATATGAATTCTA
TTTGATTGTAAAACAAAACTATAAAAATAAGTAAAAAAAT
AAAAAATAAACAGATACTTAAAATCAAAAAAAAAAAAAAA
AAAAAAAAAA
NA sequence miANG-SCR1 guide
SEQ ID NO. 92
GTAGTTCTATTAGCGCTTACTA
NA sequence miANG-SCR2 guide
SEQ ID NO. 93
ATGGATCGAGTCTCGTTATATA
HCR-hAAT promoter sequence
SEQ ID NO. 94
GGCTCAGAGGCACACAGGAGTTTCTGGGCTCACCCTGCCC
CCTTCCAACCCCTCAGTTCCCATCCTCCAGCAGCTGTTTG
TGTGCTGCCTCTGAAGTCCACACTGAACAAACTTCAGCCT
ACTCATGTCCCTAAAATGGGCAAACATTGCAAGCAGCAAA
CAGCAAACACACAGCCCTCCCTGCCTGCTGACCTTGGAGC
TGGGGCAGAGGTCAGAGACCTCTCTGGGCCCATGCCACCT
CCAACATCCACTCGACCCCTTGGAATTTCGGTGGAGAGGA
GCAGAGGTTGTCCTGGCGTGGTTTAGGTAGTGTGAGAGGA
TCTTGCTACCAGTGGAACAGCCACTAAGGATTCTGCAGTG
AGAGCAGAGGGCCAGCTAAGTGGTACTCTCCCAGAGACTG
TCTGACTCACGCCACCCCCTCCACCTTGGACACAGGACGC
TGTGGTTTCTGAGCCAGGTACAATGACTCCTTTCGGTAAG
TGCAGTGGAAGCTGTACACTGCCCAGGCAAAGCGTCCGGG
CAGCGTAGGCGGGCGACTCAGATCCCAGCCAGTGGACTTA
GCCCCTGTTTGCTCCTCCGATAACTGGGGTGACCTTGGTT
AATATTCACCAGCAGCCTCCCCCGTTGCCCCTCTGGATCC
ACTGCTTAAATACGGACGAGGACAGGGCCCTGTCTCCTCA
GCTTCAGGCACCACCACTGACCTGGGACAGTGAATCGTAA
GT
NA sequence LucANG-A
SEQ ID NO. 95
TTCTCTATCTCCAGAGCCAAAATCAAGATTTGCTATGTTA
GACGATGTAAGACATATTTCAAAAACTCAACATATTTGAT
CAGTCTTTTTATGATCTATCTCTATCTTCCAAGCCAAGAG
CACCAAGAACTACTCCCTTTCTTCAGTTGAATGTTATATC
AGGTAGTCCATGGACATTAATTCAACATCGAATAGATGGA
NA sequence LucANG-B
SEQ ID NO. 96
TACGCTACATCTAGTTGCGATTACTGGCAATGTCCCCAAT
GCAATCCCGG
NA hAAT-pri-miANG5 cassette
SEQ ID NO. 97
GGCTCAGAGGCACACAGGAGTTTCTGGGCTCACCCTGCCC
CCTTCCAACCCCTCAGTTCCCATCCTCCAGCAGCTGTTTG
TGTGCTGCCTCTGAAGTCCACACTGAACAAACTTCAGCCT
ACTCATGTCCCTAAAATGGGCAAACATTGCAAGCAGCAAA
CAGCAAACACACAGCCCTCCCTGCCTGCTGACCTTGGAGC
TGGGGCAGAGGTCAGAGACCTCTCTGGGCCCATGCCACCT
CCAACATCCACTCGACCCCTTGGAATTTCGGTGGAGAGGA
GCAGAGGTTGTCCTGGCGTGGTTTAGGTAGTGTGAGAGGA
TCTTGCTACCAGTGGAACAGCCACTAAGGATTCTGCAGTG
AGAGCAGAGGGCCAGCTAAGTGGTACTCTCCCAGAGACTG
TCTGACTCACGCCACCCCCTCCACCTTGGACACAGGACGC
TGTGGTTTCTGAGCCAGGTACAATGACTCCTTTCGGTAAG
TGCAGTGGAAGCTGTACACTGCCCAGGCAAAGCGTCCGGG
CAGCGTAGGCGGGCGACTCAGATCCCAGCCAGTGGACTTA
GCCCCTGTTTGCTCCTCCGATAACTGGGGTGACCTTGGTT
AATATTCACCAGCAGCCTCCCCCGTTGCCCCTCTGGATCC
ACTGCTTAAATACGGACGAGGACAGGGCCCTGTCTCCTCA
GCTTCAGGCACCACCACTGACCTGGGACAGTGAATCGTAA
GTCTCTGGAGCCTGACAAGGAGGACAGGAGAGATGCTGCA
AGCCCAAGAAGCTCTCTGCTCAGCCTGTCACAACCTACTG
ACTGCCAGGGCACTTGGGAATGGCAAGGTAGCAAATCTTG
ATTTTGGCTCCAAAATCAAGATTTGCTCTCTTGCTATACC
CAGAAAACGTGCCAGGAAGAGAACTCAGGACCCTGAAGCA
GACTACTGGAAGGGAGACTCCAGCTCAAACAAGGCAGGGG
TGGGGGCGTGGGATTGCTTTATTTGTGAAATTTGTGATGC
TATTGCTTTATTTGTAACCATTATAAGCTGCAATAAACAA
GTTAACAACAACAATTGCATTCATTTTATGTTTCAGGTTC
AGGGGGAGGTGTGGGAGGTTTTTTAAA
viral vector genome of hAAT-pri-miANG5
SEQ ID NO. 98
TTGGCCACTCCCTCTCTGCGCGCTCGCTCGCTCACTGAGG
CCGGGCGACCAAAGGTCGCCCGACGCCCGGGCTTTGCCCG
GGCGGCCTCAGTGAGCGAGCGAGCGCGCAGAGAGGGAGTG
GCCAACTCCATCACTAGGGGTTCCTGGAGGGGTGGAGTCG
TGACGTGAATTACGTCATAGGGTTAGGGAGGTCAGATCTG
AGCTCCATGGCGCGCCGGCTCAGAGGCACACAGGAGTTTC
TGGGCTCACCCTGCCCCCTTCCAACCCCTCAGTTCCCATC
CTCCAGCAGCTGTTTGTGTGCTGCCTCTGAAGTCCACACT
GAACAAACTTCAGCCTACTCATGTCCCTAAAATGGGCAAA
CATTGCAAGCAGCAAACAGCAAACACACAGCCCTCCCTGC
CTGCTGACCTTGGAGCTGGGGCAGAGGTCAGAGACCTCTC
TGGGCCCATGCCACCTCCAACATCCACTCGACCCCTTGGA
ATTTCGGTGGAGAGGAGCAGAGGTTGTCCTGGCGTGGTTT
AGGTAGTGTGAGAGGATCTTGCTACCAGTGGAACAGCCAC
TAAGGATTCTGCAGTGAGAGCAGAGGGCCAGCTAAGTGGT
ACTCTCCCAGAGACTGTCTGACTCACGCCACCCCCTCCAC
CTTGGACACAGGACGCTGTGGTTTCTGAGCCAGGTACAAT
GACTCCTTTCGGTAAGTGCAGTGGAAGCTGTACACTGCCC
AGGCAAAGCGTCCGGGCAGCGTAGGCGGGCGACTCAGATC
CCAGCCAGTGGACTTAGCCCCTGTTTGCTCCTCCGATAAC
TGGGGTGACCTTGGTTAATATTCACCAGCAGCCTCCCCCG
TTGCCCCTCTGGATCCACTGCTTAAATACGGACGAGGACA
GGGCCCTGTCTCCTCAGCTTCAGGCACCACCACTGACCTG
GGACAGTGAATCGTAAGTCTCTGGAGCCTGACAAGGAGGA
CAGGAGAGATGCTGCAAGCCCAAGAAGCTCTCTGCTCAGC
CTGTCACAACCTACTGACTGCCAGGGCACTTGGGAATGGC
AAGGTAGCAAATCTTGATTTTGGCTCCAAAATCAAGATTT
GCTCTCTTGCTATACCCAGAAAACGTGCCAGGAAGAGAAC
TCAGGACCCTGAAGCAGACTACTGGAAGGGAGACTCCAGC
TCAAACAAGGCAGGGGTGGGGGCGTGGGATTGCTTTATTT
GTGAAATTTGTGATGCTATTGCTTTATTTGTAACCATTAT
AAGCTGCAATAAACAAGTTAACAACAACAATTGCATTCAT
TTTATGTTTCAGGTTCAGGGGGAGGTGTGGGAGGTTTTTT
AAAGCGGCCGCAGATCTGTAGATAAGTAGCATGGCGGGTT
AATCATTAACTACAAGGAACCCCTAGTGATGGAGTTGGCC
ACTCCCTCTCTGCGCGCTCGCTCGCTCACTGAGGCCGGGC
GACCAAAGGTCGCCCGACGCCCGGGCTTTGCCCGGGCGGC
CTCAGTGAGCGAGCGAGCGCGCAGAGAGGGAGTGGCCAA
VP3 AAV5 construct (hybrid VP1) 30
SEQ ID NO. 99
MAADGYLPDWLEDTLSEGIRQWWKLKPGPPPPKPAERHKD
DSRGLVLPGYKYLGPFNGLDKGEPVNEADAAALEHDKAYD
RQLDSGDNPYLKYNHADAEFQERLKEDTSFGGNLGRAVFQ
AKKRVLEPLGLVEEPVKTAPTGKRIDDHFPKRKKARTEED
SKPSTSSDAEAGPSGSQQLQIPAQPASSLGADTMSAGGGG
PLGDNNQGADGVGNASGDWHCDSTWMGDRVVTKSTRTWVL
PSYNNHQYREIKSGSVDGSNANAYFGYSTPWGYFDFNRFH
SHWSPRDWQRLINNYWGFRPRSLRVKIFNIQVKEVTVQDS
TTTIANNLTSTVQVFTDDDYQLPYVVGNGTEGCLPAFPPQ
VFTLPQYGYATLNRDNTENPTERSSFFCLEYFPSKMLRTG
NNFEFTYNFEEVPFHSSFAPSQNLFKLANPLVDQYLYRFV
STNNTGGVQFNKNLAGRYANTYKNWFPGPMGRTQGWNLGS
GVNRASVSAFATTNRMELEGASYQVPPQPNGMTNNLQGSN
TYALENTMIFNSQPANPGTTATYLEGNMLITSESETQPVN
RVAYNVGGQMATNNQSSTTAPATGTYNLQEIVPGSVWMER
DVYLQGPIWAKIPETGAHFHPSPAMGGFGLKHPPPMMLIK
NTPVPGNITSFSDVPVSSFITQYSTGQVTVEMEWELKKEN
SKRWNPEIQYTNNYNDPQFVDFAPDSTGEYRTTRPIGTRY
LTRPL
primer forward for vector genome
quantification
SEQ ID NO. 100
GGGAGGTGTGGGAGGTTT
primer reverse for vector genome
quantification
SEQ ID NO. 101
AATGATTAACCCGCCATGCT
probe for vector genome quantification
SEQ ID NO. 102
[FAM] ACT TAT CTA CAG
ATC TGC GGC CGC T [TAMRA]
primer forward for ACTB quantification
SEQ ID NO. 103
AGGTGCACAGTAGGTCTGAACAGA
primer reverse for ACTB quantification
SEQ ID NO. 104
TGCAAAGAACACGGCTAAGTGT
target sequence for reverse transcription
miANG5 24 nts primer
SEQ ID NO. 105
TAGCAAATCTTGATTTTGGCTCCA
target sequence for probe 24 nts
miANG5 design
SEQ ID NO. 106
[FAM] TAGCAAATCTTGATTTTGGCTCCA [NFQ]
synthetic RNA oligo for miANG5 24 nts
quantification
SEQ ID NO. 107
rUrArGrCrArArArUrCrUrUrGrArUrUrUrUrGrGrC
rUrCrCrA
promoter sequence
SEQ ID NO. 108
TAAAGCAAATATTTGTGGTTATGGATTAACTCGAACTTCT
AGAAGCTGTTTGCCCACTCTATTTGCCCATCCTAGGTAGG
CGCCCTTTGGACCTTTTGCAATCCTGGCTTCTAGAAGAGC
AAACAGCAAACACATCCTAGGTAGGACTTAGCCCCTGTTT
GCTCCTCCGATAACTGGGGTGACCTTGGTTAATATTCACC
AGCAGCCTCATGCTAGCCTCGAGGATATCAGATCTGGGCG
ACTCAGATCCCAGCCAGTGGACTTAGCCCCTGTTTGCTCC
TCCGATAACTGGGGTGACCTTGGTTAATATTCACCAGCAG
CCTCCCCCGTTGCCCCTCTGGATCCACTGCTTAAATACGG
ACGAGGACAGGGCCCTGTCTCCTCAGCTTCAGGCACCACC
ACTGACCTGGGACAGTGAATCGCCACC
promoter sequence
SEQ ID NO. 109
TAAAGCAAATATTTGTGGTTATGGATTAACTCGAACTTCT
AGAAGCTGTTTGCCCACTCTATTTGCCCATCCTAGGTAGG
CGCCCTTTGGACCTTTTGCAATCCTGGCTTCTAGAAGAGC
AAACAGCAAACACATCCTAGGTAGGACTTAGCCCCTGTTT
GCTCCTCCGATAACTGGGGTGACCTTGGTTAATATTCACC
AGCAGCCTCATGCTAGCCTCGAGGATATCAGATCTTCATC
TATTTCCTGCCCACATCTGGTATAAAAGGAGGCAGTGGCC
CACAGAGGAGCACAGCTGTGCCACC
promoter sequence
SEQ ID NO. 110
TTAATATTTAACATCCTAGCACAGCTTCACTTCCAGGTAT
GACCTTTGAACCTCTTCTAGAAGGGTAATTATTAACCTAG
CTAGGTATGACCTTCGAACCTCTTCTAGAAGTGAAGCTGG
GCATATAAACAGGGGCAAGGCACAGACTCATAGCAGAGCA
ATTACCACCAAGCCTGGAATAGCTGCAGCCACC
promoter sequence
SEQ ID NO. 111
GGTTAATAATTACCCTTCTAGGATTGAGTCACTTCTAGAA
GCTGGACTTTGGACTCATCCTAGAAGTCACTTCCTCTTTT
TTACCTAGAAGAGGTTCAAAGGTCATACCTAGCATAGCTT
CACTTCTAGAAGGGTAATTATTAACCGGGCATATAAAACA
GGGGCAAGGCACAGACTCATAGCAGAGCAATCACCACCAG
GCCTGGAATAACTGCAGCCAC
C
promoter sequence
SEQ ID NO. 112
CATAGCTTCACTTCTAGAAGAGGTCAGGGTGACCTGGGCC
TACCTAGCTAGGTTAATAATTACCCTTCTAGAAGTGACTC
AATCCTAGAAGCCGGAAGTGGCATCCTAGAAGAGGTTCAA
AGGTCATACCTAGGTAAAAAAGAGGAAGTGACTTCTAGGA
TAAGGAAGTACTTCTAGAAGTACTTCCTTATCCTAGCATA
GCTTCACTTCTAGAAGAGGTTCAAAGGTCATACCTAGGTA
TGACCTTTGAACCTCTTCTANAAGTTAATATTTAACATCC
TAGAAGGGTAATTATTAACCTAGCAAGGCTGACTACACGA
GCACATATCAGCGCGTCGACGATATCAGACCTGGGCATAT
AAAACAGGGGCAAGGCACAGACTCATAGCAGAGCAATCAC
CACCAAGCCTGGAATAACTGCAGCCACCATGG
promoter sequence
SEQ ID NO. 113
TTTCTCTGGCCTAACTGGCCGGTACCGTCGACTGTGCTCG
GACCTGTAGATGCTAGTCTAGAAGAGGTTCAAAGGTCATA
CCTAGGATAAGGAAGTACTTCTAGGTAGGCCCAGGTCACC
CTGACCTCTTCTAGGATAAGGAAGTACTTCTAGAAGAGGT
CAGGGTGACCTGGGCCTACCTAGAAGTACTTCCTTATCCT
AGGTATGACCTTTGAACCTCTTCTAGACTAGCATCTACAG
GTCCGAGCACAGTCGACGGTACCGGCCAGTTAGGCCAGAG
AAATGTTCTGNCACCTG
promoter sequence
SEQ ID NO. 114
TAGTAGGGCAAAGGTCACTTCTAGAAGCCGGAAGTGGCAT
CCTAGAAGTGACTCAATCCTAGAAGAGGTCAGGGTGACCT
GGGCCTACCTAGAAGTGACTCAATCCTAGGATGTTAAATA
TTAACTTCTAGTAGCAAGGCTGACTACACGAGCACATATC
AACGCGTCGACGATATCAGATCTGGGCATATAAAACAGGG
GCAAGGCACAGACTCATAGCAGAGCAATCACCACCAAGCC
TGGAATAACTGCAGCCACCATGG
derivative of SEQ ID NO: 94
SEQ ID NO. 115
AAGCAAATATTTGTGGTTATGGATTAACTCGAACTGTTTG
CCCACTCTATTTGCCCGGCGCCCTTTGGACCTTTTGCAAT
CCTGGAGCAAACAGCAAACACGACTCAGATCCCAGCCAGT
GGACTTAGCCCCTGTTTGCTCCTCCGATAACTGGGGTGAC
CTTGGTTAATATTCACCAGCAGCCTCCCCCGTTGCCCCTC
TGGGGCATATAAAACAGGGGCAAGGCACAGACTCATAGCA
GAGCAATCACCACCAAGCCTGGAATAACTGCAGCCACC
nucleic acid sequence encoding a
modified miR-451
SEQ ID NO. 124
CTCTGGAGCCTGACAAGGAGGACAGGAGAGATGCTGCAAG
CCCAAGAAGCTCTCTGCTCAGCCTGTCACAACCTACTGAC
TGCCAGGGCACTTGGGAATGGCAAGGTAGCAAATCTTGAT
TTTGGCTCCAAAATCAAGATTTGCTCTCTTGCTATACCCA
GAAAACGTGCCAGGAAGAGAACTCAGGACCCTGAAGCAGA
CTACTGGAAGGGAGACTCCAGCTCAAACAAGGCAGGGGTG
GGGGCGTGGGAT,
target sequence for reverse transcription
miANG5 23 nts variant T primer
SEQ ID NO. 125
TAGCAAATCTTGATTTTGGCTCT
target sequence for probe miANG5 23 nts
variant T design
SEQ ID NO. 126
TAGCAAATCTTGATTTTGGCTCT
synthetic RNA oligo for miANG5 23 nts
variant T quantification
SEQ ID NO. 127
rUrArGrCrArArArUrCrUrUrGrArUrUrUrUrGrGrC
rUrCrU

1. A nucleic acid comprising a nucleic acid sequence encoding an RNA molecule comprising a first RNA sequence and a second RNA sequence, wherein the first RNA sequence comprises a sequence that is substantially complementary to a target RNA sequence comprised in an RNA encoded by an Angiopoietin-like 3 (ANGPTL3) gene, wherein the sequence substantially complementary to the target RNA sequence has at least 19 nucleotides, and wherein the RNA molecule comprises a hairpin, a double stranded RNA (dsRNA), small interfering RNA (siRNA), or microRNA (miRNA).

2. The nucleic acid according to claim 1, wherein the hairpin is a short hairpin RNA (shRNA) or long hairpin RNA (lhRNA).

3. The nucleic acid according to claim 1, wherein the RNA molecule comprises miR451.

4. The nucleic acid according to claim 1, wherein the sequence substantially complementary to the target RNA sequence has at least 22 nucleotides.

5. The nucleic acid according to claim 1, wherein the sequence substantially complementary to the target RNA sequence has at most 30 nucleotides.

6. The nucleic acid according to claim 1, wherein the target RNA sequence comprises an RNA sequence encoded by part of at least one exon comprised in the ANGPTL3 gene.

7. The nucleic acid according to claim 6, wherein the exon is exon 1, exon 3, exon 5 or exon 6.

8. The nucleic acid according to claim 1, wherein part of the at least one exon comprised in the ANGPTL3 gene comprises SEQ ID NO. 3, SEQ ID NO. 4, SEQ ID NO. 5, SEQ ID NO. 6, or SEQ ID NO. 7.

9. The nucleic acid according to claim 1, wherein the sequence substantially complementary to the target RNA sequence is selected from the group consisting of SEQ ID NOs. 8-25.

10. The nucleic acid according to claim 1, wherein the sequence substantially complementary to the target RNA sequence is selected from the group consisting of SEQ ID NO. 11, SEQ ID NO. 12, SEQ ID NO. 16, SEQ ID NO. 17, SEQ ID NO. 20 and SEQ ID NO. 25.

11. The nucleic acid according to claim 1, which is a DNA molecule.

12. A DNA expression cassette, comprising a nucleic acid according to claim 1, wherein the DNA expression cassette further comprises a promoter and a poly A tail, and wherein the DNA expression cassette is flanked by two Inverted Terminal Repeat (ITR)s.

13. The DNA expression cassette according to claim 12, wherein the promoter comprises a liver-specific promoter.

14. An AAV gene therapy vehicle, comprising a DNA expression cassette according to claim 11.

15. The AAV gene therapy vehicle according to claim 14, wherein a capsid of the AAV gene therapy vehicle comprises an AAV5 capsid protein sequence.

16. A composition comprising the AAV gene therapy vehicle according to claim 14 and an excipient.

17. The composition according to claim 16, further comprising at least one molecule which reduces and/or inhibits cholesterol levels in plasma, low-density lipoprotein cholesterol (LDL-C) levels, and/or atherosclerotic lesions.

18. The composition according to claim 17, wherein the atherosclerotic lesions comprise severe atherosclerotic lesions.

19. The composition according to claim 17, wherein the at least one molecule comprises at least one of statins.

20. The composition according to claim 17, wherein the at least one molecule is selected from the group consisting of Atorvastatin, Cerivastatin, Fluvastatin, Lovastatin, Mevastatin, Pitavastatin, Pravastatin, Rosuvastatin, and Simvastatin.

21. The composition according to claim 20, wherein the at least one molecule comprises Atorvastatin and/or Simvastatin.

22. A method of decreasing and/or inhibiting cholesterol levels in plasma, phospholipids levels, atherosclerosis lesions, triglyceride (TG) levels, total cholesterol (TC) levels, and/or low-density lipoprotein cholesterol (LDL-C) levels, comprising administering to a subject in need thereof a composition according to claim 16.
